(19) United States
(12) Patent Application Publication
(10) Pub. No.: US 2011/0154532 A1
(43) Pub. Date: Jun. 23, 2011

(54) COMPOSITIONS AND METHODS RELATED TO SILICON TRANSPORT

(75) Inventors: Richard Bélanger, Quebec (CA); Wilfried Rémus-Borel, Quebec (CA); Caroline Grégoire, Sainte-Marie (CA)

(73) Assignee: Université Laval, Quebec, QC (CA)

(21) Appl. No.: 12/934,255

(22) PCT Filed: Mar. 24, 2009
(86) PCT No.: PCT/CA2009/000373

§ 371 Date: Feb. 16, 2011

Related U.S. Application Data
(60) Provisional application No. 61/070,528, filed on Mar. 24, 2008.

(51) Int. Cl. A01H 1/06; C07H 21/04; C12N 15/82; C12N 5/10; A01H 5/10; C07K 14/415; A01H 5/00

(52) U.S. Cl. 800/278; 536/23.6; 435/320.1; 435/419; 435/415; 800/298; 530/370; 800/312

(57) ABSTRACT

Based on our identification of silicon influx and efflux transporter genes in plants known to take up silicon efficiently including wheat, horsetail, oat, sorghum, and barley, the present invention features polynucleotides encoding silicon transporters; vectors, cells, and plants including such polynucleotides, and methods for making such plants. The invention also features silicon transporter polypeptides and fragments thereof. Plants expressing heterologous silicon transporters may exhibit both increased silicon uptake and increased resistance to biotic and abiotic stresses. In particular, plants such as soybean expressing silicon transporters may exhibit increased resistance to pathogens such as rust.

### BACKGROUND OF THE INVENTION

The invention relates to compositions and methods which may be useful for increasing silicon uptake and increasing resistance to biotic and abiotic stresses in plants such as soybean.

Biotic and abiotic stresses on plants cause billions of dollars worth of damage to crops each year. For example, Soybean rust, a disease caused by the Phakopsora pachyrhizi fungus, resulted in approximately $1 billion worth of damage in Brazil in 2003. This disease has now begun to spread into the United States, the largest producer of soybean worldwide.

While the rust can be treated using chemical fungicides, doing so is expensive, potentially damaging to the environment, and may only be partially effective. Accordingly, there is a need for additional or improved methods for protecting plants against biotic as well as abiotic stresses. Prevention or control of soybean rust is one of the most important applications in this regard.

### SUMMARY OF THE INVENTION

We have discovered silicon influx and efflux transporter genes in plants known to take up silicon efficiently, including wheat, horsetail, sorghum, oat, and barley. The encoded transporter proteins increase resistance to biotic and abiotic stressors when expressed in a plant (e.g., soybean). The present invention thus features polynucleotides encoding silicon transporters; vectors, cells, and plants including such polynucleotides; and methods for making such plants. The invention also features silicon transporter polypeptides and fragments thereof. Particularly useful are soybean plants transformed with the silicon transporters described herein, where expression of the silicon transporter results in increased resistance to soybean rust.

Accordingly, in a first aspect, the invention features a substantially pure polynucleotide including a nucleic acid sequence substantially identical (e.g., at least 85%, 90%, 95%, 96%, 97%, 98%, 99%, 99.5%, or 100% identical) to a sequence selected from the group consisting of SEQ ID NOS:4, 9, 12, 13, 14, 15, 33, 52, 67, nucleotides 124-919 of SEQ ID NO:2l, nucleotides 146-694 of SEQ ID NO:22, and nucleotides 124-1014 of SEQ ID NO:32, or a fragment thereof. The invention also features a polynucleotide including a nucleic acid sequence that encodes a polypeptide substantially identical to a sequence selected from the group consisting of SEQ ID NOS:5, 6, 34-38, 60, and 68, or a fragment thereof. In other embodiments, the nucleic acid sequence is modified to contain one or more (e.g., at least 2, 3, 4, 5, 8, 10, 15) mutations, deletions, insertions, or a combination thereof. The modified nucleic acid sequence may encode a polypeptide having increased or decreased silicon transport when expressed in a cell. The polypeptide may have a mutation at the position corresponding to position 132 of the wheat SIIT1 sequence (SEQ ID NO:37). In certain embodiments, the mutation is a threonine to alanine mutation). In certain embodiments, the polynucleotide is substantially identical to SEQ ID NO:50 or the polynucleotide encodes a polypeptide substantially identical to SEQ ID NO:51. In some embodiments, expression of the polypeptide encoded by the polynucleotide of the first aspect in a cell increases or is capable of increasing silicon transport into the cell.

In another aspect, the invention features a substantially pure polynucleotide including a nucleic acid sequence substantially identical (e.g., at least 85%, 90%, 95%, 96%, 97%, 98%, 99%, 99.5%, or 100% identical) to a nucleotide sequence selected from the group consisting of SEQ ID NOS:29, 71, and 73, or a fragment thereof. The invention also features a substantially pure polynucleotide including a nucleic acids sequence that encodes a polypeptide substantially identical to an amino acid sequence selected from the group consisting of SEQ ID NOS:31, 72, and 74, or a fragment thereof. In some embodiments, expression of the polypeptide encoded by the polynucleotide in a cell increases or is capable of increasing silicon transport from the cell.

In another aspect, the invention features a substantially pure polynucleotide including a nucleic acid sequence substantially identical (e.g., at least 85%, 90%, 95%, 96%, 97%, 98%, 99%, 99.5%, or 100% identical) to a nucleotide sequence selected from the group consisting of SEQ ID NOS:56, 58, and 59, or a fragment thereof. The invention also features a substantially pure polynucleotide including a nucleic acid sequence that encodes a polypeptide substantially identical to a sequence selected from the group consisting of SEQ ID NOS:63, 65, and 66, or a fragment thereof. In some embodiments, expression of the polypeptide encoded by the polynucleotide in a cell increases or is capable of increasing silicon transport into the cell.

In any of the above aspects, the polynucleotide may be less than 1,000, 500, 100, 50, 30, 20, 15, 10, 8, 6, 5, 4, 3, or 2 kb in length. The polynucleotide may be operably linked to a promoter, for example, a promoter capable of expression in a plant cell. The promoter may be time-dependent, cell specific (e.g., root cells), or tissue specific (e.g., in any tissue described herein). The promoter may be constitutive or inducible, for example, under environmental conditions such any abiotic or biotic stress (e.g., those described herein). The invention also features a vector including a polynucleotide of the invention. The vector may further include a second polynucleotide. In one embodiment, the second polynucleotide encodes a silicon efflux transporter or a fragment thereof (e.g., a polynucleotide substantially identical to a sequence selected from the group consisting of SEQ ID NOS:28, 29, 71, and 73, a polynucleotide encoding a polypeptide substantially identical to a sequence selected from the group consisting of SEQ ID NOS:30, 31, 72, and 74, or a fragment thereof). In another embodiment, the second polynucleotide encodes a silicon influx transporter or a fragment thereof (e.g., a polynucleotide substantially identical to a sequence selected from the group consisting of SEQ ID NOS:3, 4, 9, 12, 13, 14, 15, 33, 50, 52, 53, 55, 56, 57, 58, 59, 67, nucleotides 124-919 of SEQ ID NO:21, nucleotides 146-694 of SEQ ID NO:22, and nucleotides 124-1014 of SEQ ID NO:32, a polynucleotide encoding a polypeptide substantially identical to an amino acid sequence selected from the group consisting of SEQ ID NOS:5, 6, 34-38, 51, 54, 60, 61, 62, 63, 64, 65, 66, and 68, or a fragment thereof). The invention also features a cell such as a plant cell (e.g., a soybean cell or a cell from any plant described herein), a bacterial cell, or any cell described herein including the vector. The cell may, in some embodiments, be part of a plant seed or a tissue from a plant (e.g., any described herein).

The invention also features a polypeptide, or fragment thereof, encoded by any of the polynucleotides described herein. The polypeptide may be substantially pure or may be expressed in a cell recombinantly.

In another aspect, the invention features a plant (e.g., soybean or any plant described herein), plant tissue, or seed including one or more heterologous polynucleotides including a nucleic acid sequence substantially identical to a nucleic acid sequence encoding a silicon influx transporter or a fragment thereof (e.g., a nucleic acid sequence substantially identical to a sequence selected from the group consisting of SEQ ID NOS:3, 4, 9, 12, 13, 14, 15, 33, 50, 52, 53, 67, nucleotides 124-919 of SEQ ID NO:21, nucleotides 146-694 of SEQ ID NO:22, and nucleotides 124-1014 of SEQ ID NO:32, or a fragment thereof) or a nucleic acid sequence encoding a polypeptide substantially identical to an amino acid sequence SEQ ID NOS:5, 6, 34-38, 51, 54, 60, 61, and 68, or a fragment thereof. The polypeptide encoded by the heterologous polynucleotide may increase or be capable of increasing the transport of silicon into at least one tissue or cell (e.g., root cells) within the plant upon expression. The plant, plant tissue, or seed may further include a second heterologous polynucleotide substantially identical to a polynucleotide encoding a silicon efflux transporter, a silicon influx transporter, or a fragment thereof. The second heterologous polynucleotide may be substantially identical to (a) a nucleic acid sequence selected from the group consisting of SEQ ID NO:28, 29, 71, and 73, (b) a nucleic acid sequence encoding an amino acid sequence selected from the group consisting of SEQ ID NO:30, 31, 72, and 74, or (c) a fragment thereof. In other embodiments, the second heterologous polynucleotide is substantially identical to (a) the nucleic acid sequence of SEQ ID NO:55, 56, 57, 58, or 59, (b) a nucleic acid sequence encoding the amino acid sequence of SEQ ID NO:62, 63, 64, 65, or 66, or (c) a fragment thereof. The plant may exhibit increased resistance to one or more biotic or abiotic stress (e.g., those described herein). In one embodiment, the plant is a soybean plant exhibiting increased resistance to soybean rust, or a tissue or seed from such a plant.

In another aspect, the invention features a plant (e.g., soybean or any plant described herein), plant tissue, or seed including one or more heterologous polynucleotides including a nucleic acid sequence substantially identical to a nucleic acid sequence encoding a silicon efflux transporter or a fragment thereof. The polynucleotide may include a sequence substantially identical to (a) a nucleic acid sequence selected from the group consisting of SEQ ID NOS:28, 29, 71, and 73 or (b) a nucleic acid sequence encoding an amino acid sequence selected from the group consisting of SEQ ID NOS:30, 31, 72, and 74, or (c) a fragment thereof. The heterologous polynucleotide may encode a polypeptide that increases or is capable of increasing transport of silicon from at least one tissue or cell (e.g., a root cell) within the plant upon expression. The plant, plant tissue, or seed may further include a second heterologous polynucleotide sequence substantially identical to a nucleic acid sequence encoding a silicon influx transporter, or a fragment thereof. The second polynucleotide may be substantially identical to (a) a nucleic acid sequence selected from the group consisting of SEQ ID NOS:3, 4, 9, 12, 13, 14, 15, 33, 50, 52, 53, 67, nucleotides 124-919 of SEQ ID NO:21, nucleotides 146-694 of SEQ ID NO:22, and nucleotides 124-1014 of SEQ ID NO:32, (b) a nucleic acid sequence encoding an amino acid sequence selected from the group consisting of SEQ ID NOS:5, 6, 34-38, 51, 54, 60, 61, and 68, or (c) a fragment thereof. In other embodiments, the second heterologous polynucleotide is substantially identical to (a) a nucleic acid sequence selected from the group consisting of SEQ ID NOS:55, 56, 57, 58, and 59, (b) a nucleic acid sequence encoding an amino acid sequence selected from the group consisting of SEQ ID NOS:62, 63, 64, 65, and 66, or (c) a fragment thereof. The plant, plant tissue, or seed may exhibit increased resistance to one or more biotic or abiotic stress (e.g., those described herein). In one embodiment, the plant is a soybean plant exhibiting increased resistance to soybean rust, or a tissue or seed from such a plant.

In yet another aspect, the invention features a plant (e.g., soybean or any plant described herein), plant tissue, or seed including a heterologous polynucleotide substantially identical to a nucleic acid sequence encoding a silicon influx transporter or a fragment thereof. The polynucleotide may include a nucleic acid sequence substantially identical (e.g., at least 85%, 90%, 95%, 96%, 97%, 98%, 99%, 99.5%, or 100% identical) to (a) a nucleic acid sequence selected from the group consisting of SEQ ID NOS:55, 56, 57, 58, and 59, (b) a nucleic acid sequence encoding an amino acid sequence selected from the group consisting of SEQ ID NO:62, 63, 64, 65, and 66, or (c) a fragment thereof. The polypeptide encoded by the heterologous polynucleotide may increase or be capable of increasing the transport of silicon into at least one tissue or cell (e.g., root, stem, or leaf cells) within the plant upon expression. The plant, plant tissue, or seed may further include a second heterologous sequence. The second polynucleotide may be silicon influx transporter (e.g., a polynucleotide substantially identical to (a) a nucleic acid sequence selected from the group consisting of SEQ ID NOS:3, 4, 9, 12, 13, 14, 15, 33, 50, 52, 53, 67, nucleotides 124-919 of SEQ ID NO:21, nucleotides 146-694 of SEQ ID NO:22, and nucleotides 124-1014 of SEQ ID NO:32, (b) a nucleic acid sequence encoding an amino acid sequence selected from the group consisting of SEQ ID NO:5, 6, 34-38, 51, 54, 60, 61, and 68, or (c) a fragment thereof). In certain embodiments, the plant is transformed with a third heterologous polynucleotide, e.g., a polynucleotide encoding a silicon efflux transporter. In other embodiments, the second heterologous polynucleotide encodes silicon efflux transporter. The polynucleotides encoding a silicon efflux transporter can be a polynucleotide substantially identical to (a) a nucleic acid sequence selected from the group consisting of SEQ ID NO:28, 29, 71, and 73, (b) a nucleic acid sequence encoding an amino acid sequence selected from the group consisting of SEQ ID NO:30, 31, 72, and 74, or (c) a fragment thereof.

The invention also features methods for generating any of the plants, plant tissues, or seeds described above. In one aspect, the method includes (a) providing a first vector including a polynucleotide substantially identical to a nucleic acid sequence encoding a silicon influx transporter or a fragment thereof (e.g., any of those described above); (b) transforming a plant cell (e.g., a soybean cell or a cell from any plant described herein) with the vector; and (c) growing a plant from the cell, where the plant expresses the polynucleotide, thereby generating a plant with increased silicon uptake. The transformation may be performed using any method known in the art (e.g., any method described herein). The vector may include a second polynucleotide including a nucleic acid sequence substantially identical to a silicon efflux transporter (e.g., any of those described above), or a fragment thereof.

In another aspect, the invention also features a method of generating a plant, plant tissues, or plant seeds with increased silicon transport. The method includes (a) providing a first vector including a polynucleotide substantially identical to a nucleic acid sequence encoding a silicon transporter, or a fragment thereof (e.g., an influx transporter (e.g., an SIIT1 or SIIT2) or an efflux transporter, such as any of those described above); (b) transforming a plant cell (e.g., a soybean cell or cell from any plant described herein) with the vector; and (c) growing a plant from the cell, where the plant expresses the polynucleotide, thereby generating a plant with increased silicon transport. The vector may further include a second polynucleotide substantially identical to a nucleic acid encoding a silicon influx transporter (e.g., any of those described above), or a fragment thereof.

In either of the two previous methods, the second polynucleotide may alternatively be included in a second vector, which is transformed (e.g., simultaneously with or sequentially to) the first vector. In either of the previous two methods, the method may further include step (d) generating seeds from the plant or harvesting at least one tissue from the plant. In one embodiment, the first polynucleotide is a silicon efflux transporter and the second polynucleotide is a silicon influx transporter.

In the aspects directed to plants, plant tissues, and seeds or related methods, the plant tissue may be, for example, root, fruit, ovule, male tissue, seed, integument, tuber, stalk, pericarp, leaf, stigma, pollen, anther, petal, sepal, pedicel, silique, and stem. Seed tissues include embryo, endosperm, and seed coat.

By “substantially pure polynucleotide” is meant a nucleic acid (e.g., a DNA or an RNA molecule) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “substantially pure polypeptide” is meant a polypeptide that has been separated from the components that naturally accompany it. Typically, the polypeptide is substantially pure when it is at least 30%, 50%, 60%, 70%, 80%, 90% 95%, or even 99%, by weight, free from the proteins and naturally-occurring organic molecules with which it is naturally associated. A substantially pure polypeptide may be obtained by extraction from a natural source, by expression of a recombinant nucleic acid in a cell that does not normally express that protein, or by chemical synthesis.

By “transformed cell” is meant a cell into which (or into an ancestor of which) has been introduced, by means of recombinant DNA techniques, a DNA molecule, for example, a DNA molecule encoding a silicon influx or efflux transporter or any of the nucleic acids described herein.

By “fragment” of a polynucleotide or amino acid sequence is meant at least 10, 15, 20, 25, 30, 50, 75, 100, 250, 300, 400, or 500 contiguous nucleic acids or amino acids of any of a longer sequence (e.g., a sequence described herein).

The term “substantial identity” as applied to amino acid sequences denotes a characteristic of a polypeptide, wherein the peptide comprises a sequence that has at least 60% 70%, 80%, 85%, 90%, 95%, 96%, 97%, 98%, 99%, or 100% sequence identity, to another sequence (e.g., any of the sequences of FIG. 1, or a fragment thereof).

The term “substantial identity” as applied to nucleic acid sequences denotes a characteristic of a polynucleotide sequence, wherein the polynucleotide comprises a sequence that has at least 50 percent, preferably 50%, 60%, 65%, 70%, 75%, 80%, 85%, 90%, 95%, 96%, 97%, 98%, 99%, or 100% identical as compared to a reference (e.g., any of the sequences described herein).

Another indication that nucleotide sequences are substantially identical is if two molecules hybridize to each other under stringent conditions.

Stringent conditions are sequence-dependent and will be different in different circumstances. Generally, stringent conditions are selected to be about 5° C. to about 20° C., usually about 10° C. to about 15° C., lower than the thermal melting point (Tₘ) for the specific sequence at a defined ionic strength and pH. The Tₘ is the temperature (under defined ionic strength and pH) at which 50% of the target sequence hybridizes to a matched probe. Typically, stringent conditions will be those in which the salt concentration is about 0.02 molar at pH 7 and the temperature is at least about 60° C. For instance in a standard Southern hybridization procedure, stringent conditions will include an initial wash in 6×SSC at 42° C. followed by one or more additional washes in 0.2×SSC at a temperature of at least about 55° C., typically about 60° C., and often about 65° C.

Nucleotide sequences are also substantially identical for purposes of this invention when said nucleotide sequences encode polypeptides and/or proteins which are substantially identical. Thus, where one nucleic acid sequence encodes essentially the same polypeptide as a second nucleic acid sequence, the two nucleic acid sequences are substantially identical even if they would not hybridize under stringent conditions due to degeneracy permitted by the genetic code (see, Darnell et al., Molecular Cell Biology, Second Edition Scientific American Books W. H. Freeman and Company New York, 1990 for an explanation of codon degeneracy and the genetic code). Protein purity or homogeneity can be indicated by a number of means well known in the art, such as polyacrylamide gel electrophoresis of a protein sample, followed by visualization upon staining. For certain purposes high resolution may be needed and HPLC or a similar means for purification may be used.

By a polypeptide which “increases silicon transport” into or from a cell is meant a polypeptide whose expression in that cell results in increase (e.g., by at least 5%, 10%, 25%, 50%, 100%, 200%, 300%, 500%, 1,000%, 5,000%, or 10,000%) in the rate of silicon or germanium transport through the cell membrane (e.g., into or out of the cell) as compared to a cell lacking the polypeptide, but does not substantially disrupt the cell membrane or increase transport of other molecules (e.g., glycerol) in a non-specific manner. A “silicon influx transporter” or a “silicon efflux transporter” is a polypeptide that is able to increase silicon transport into or out of a cell, respectively.

Other features and advantages of the invention will be apparent from the following Detailed Description, the drawings, and the claims.

### BRIEF DESCRIPTION OF THE DRAWINGS

FIG. 1a-1vvv is a list of the sequences described herein (SEQ ID NOS:1-74).

FIG. 2 is an alignment of SIIT1 and SIIT2 predicted amino acid sequences. Identical amino acids are marked in black; similar amino acids are marked in gray.

FIG. 3 is an alignment of SIET1 predicted amino acid sequences. Identical amino acids are marked in black; similar amino acids are marked in gray.

FIG. 4 is a set of graphs showing quantification of silicon concentration in oocytes after 0, 15, 30, or 60 minutes of incubation in a solution without Si or with 1.7 mM Si. Control oocytes were injected with water. Rice and wheat SIIT1 were tested for their ability to transport Si.

FIG. 5 is a set of graphs showing quantification of silicon concentration in oocytes after 0, 15, 30, or 60 minutes of incubation in a solution without Si or with 1.7 mM Si. Control oocytes were injected with water. “Lsi−” indicates oocytes injected with mutated SIIT1 cRNA, and “Lsi+” indicates oocytes injected with wild-type SIIT1 cRNA.

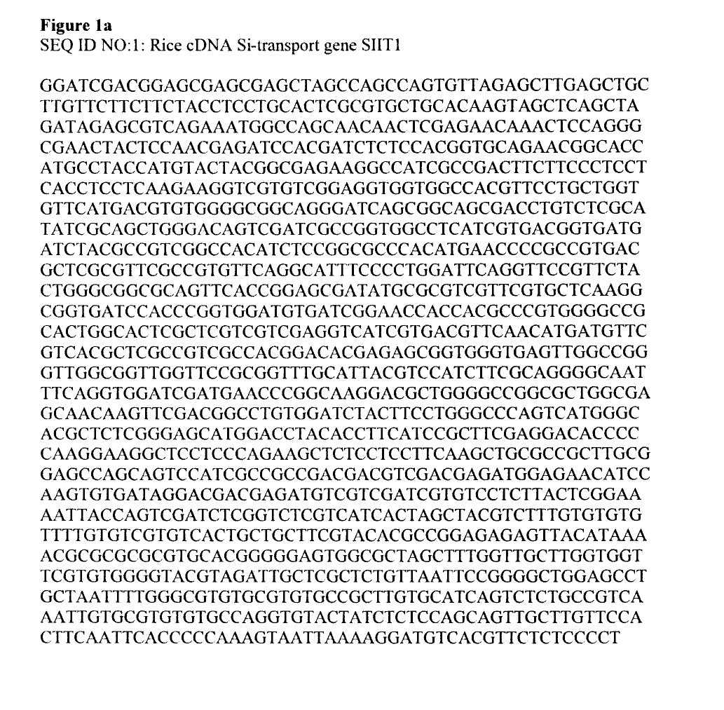

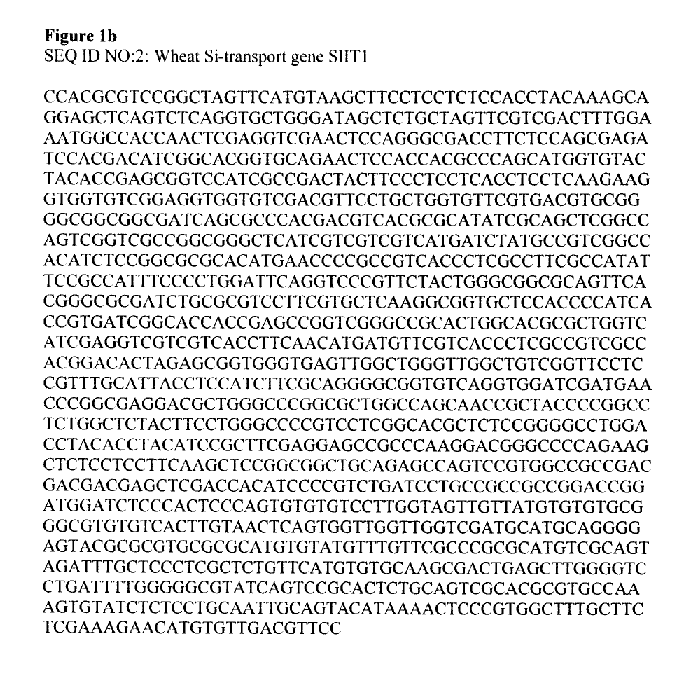

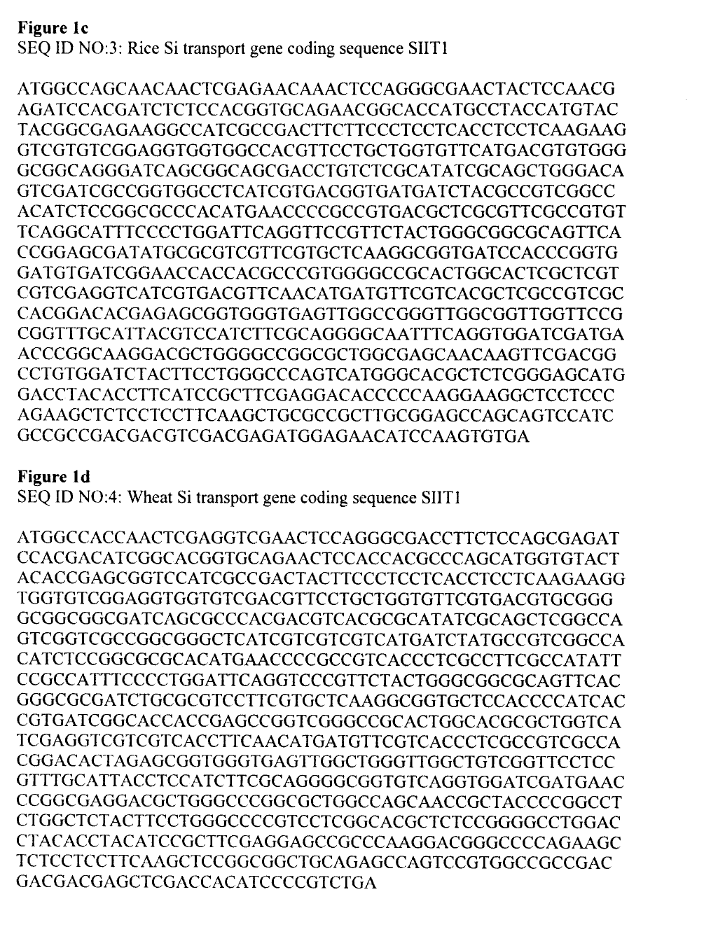

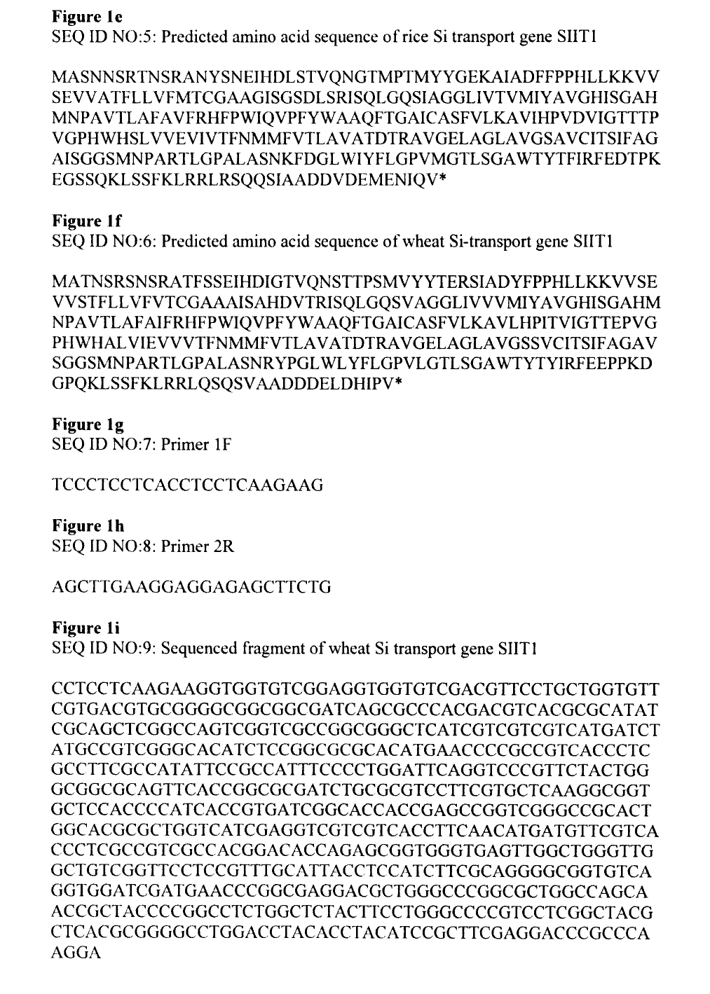

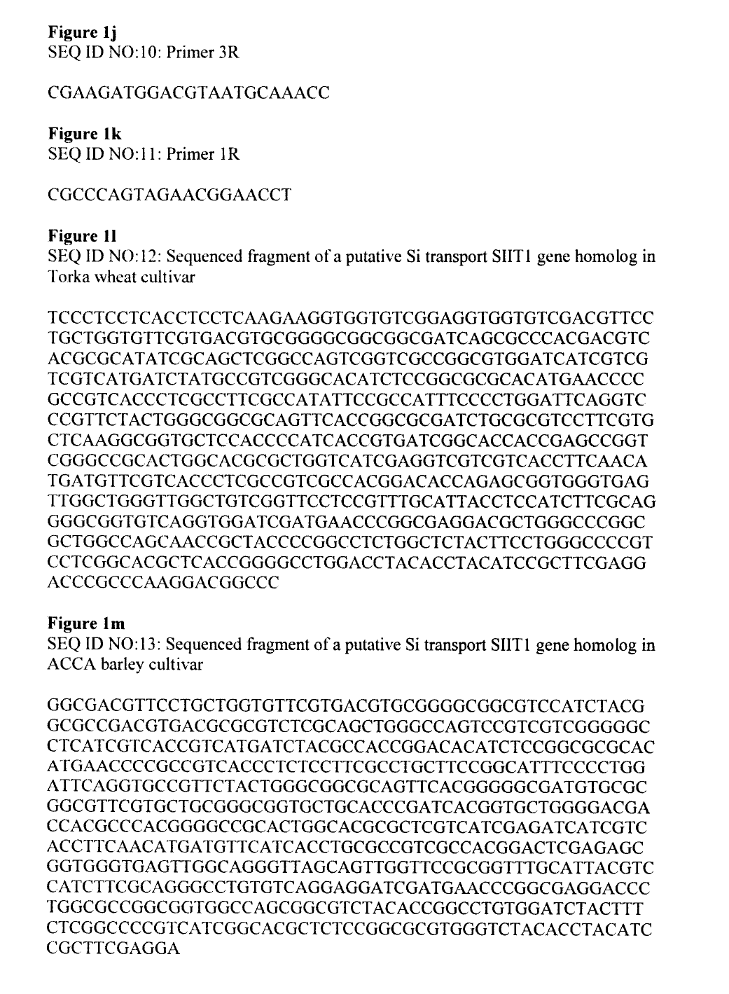

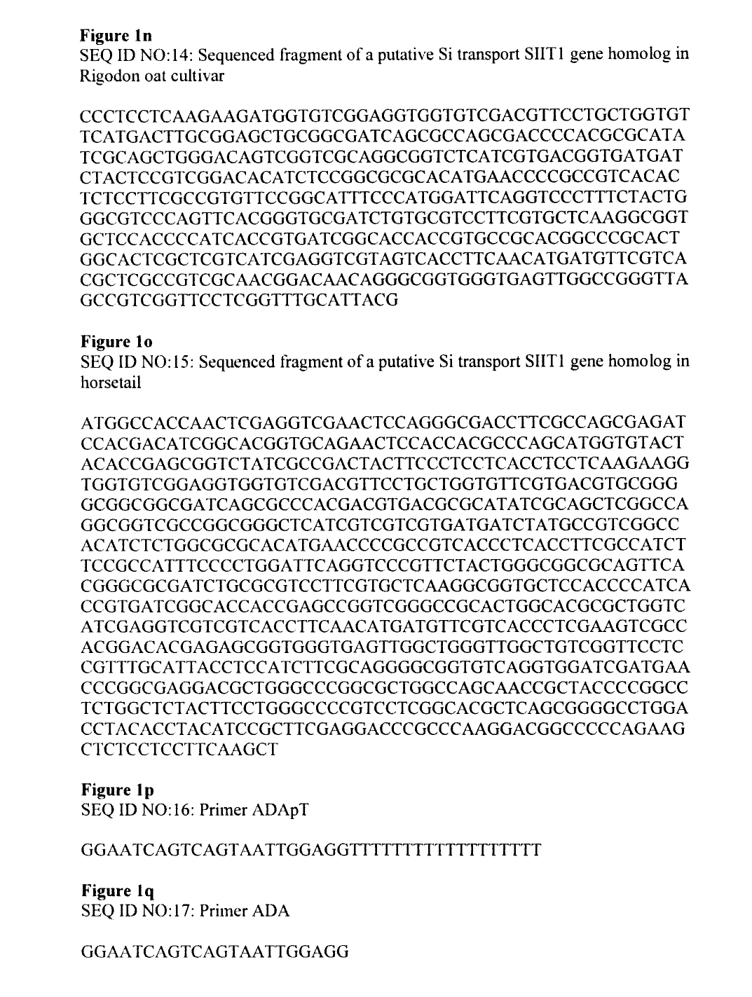

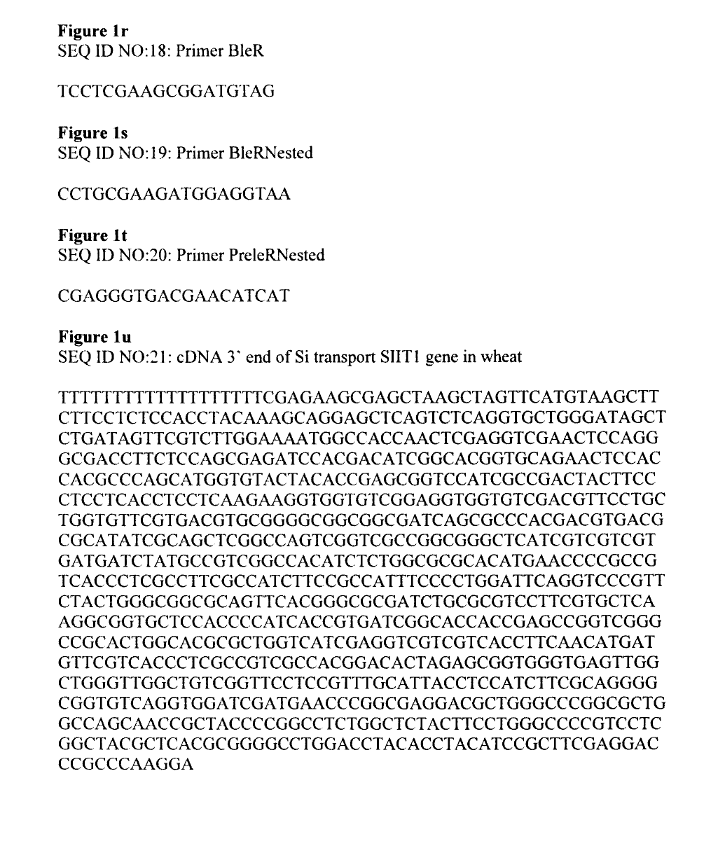

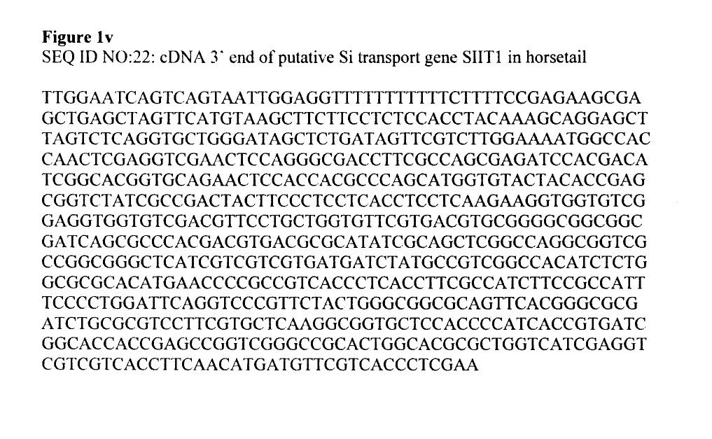

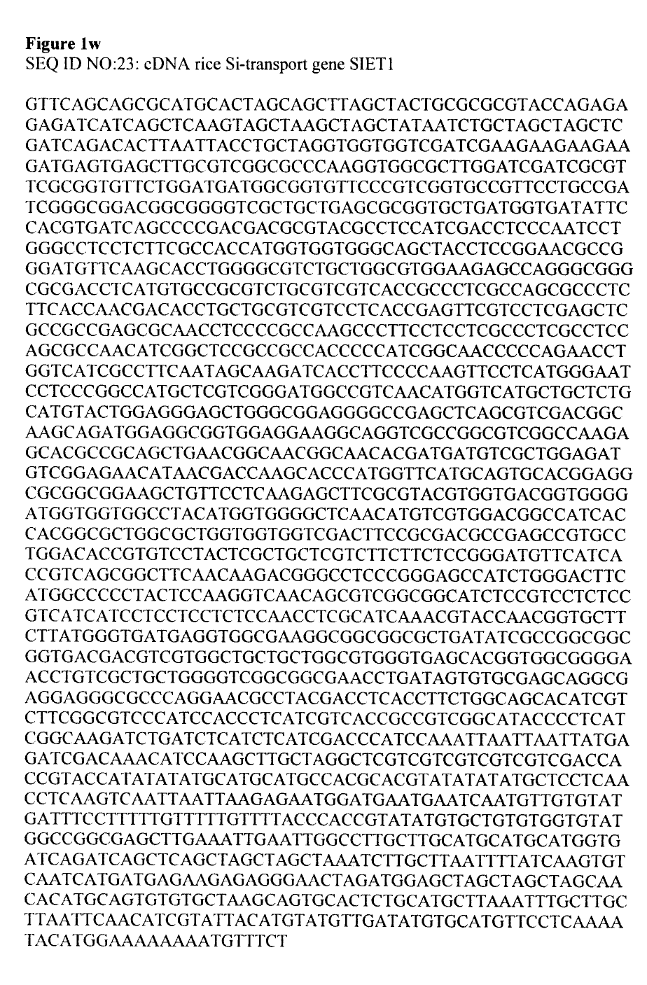

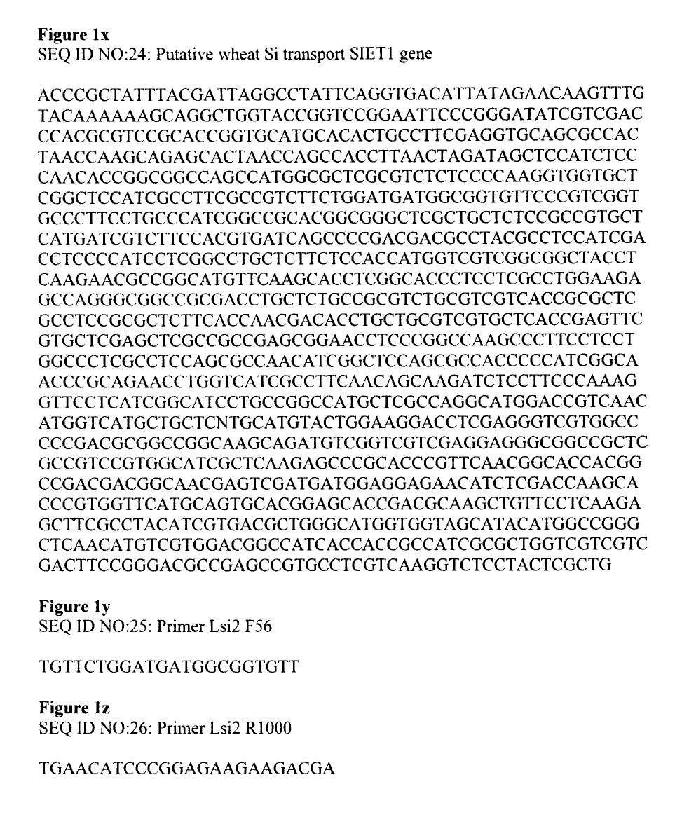

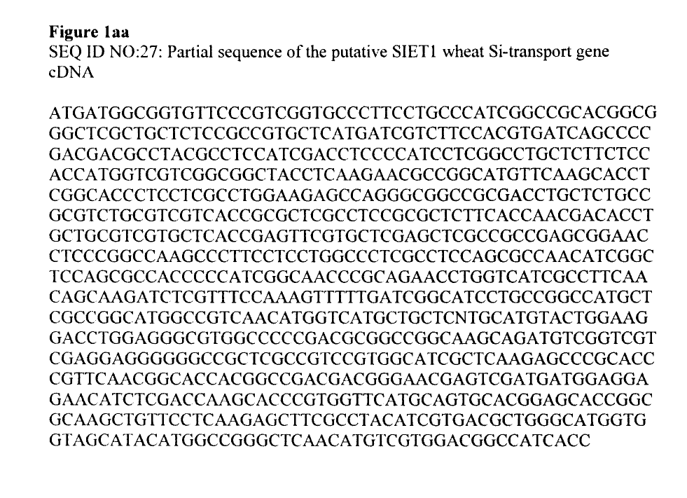

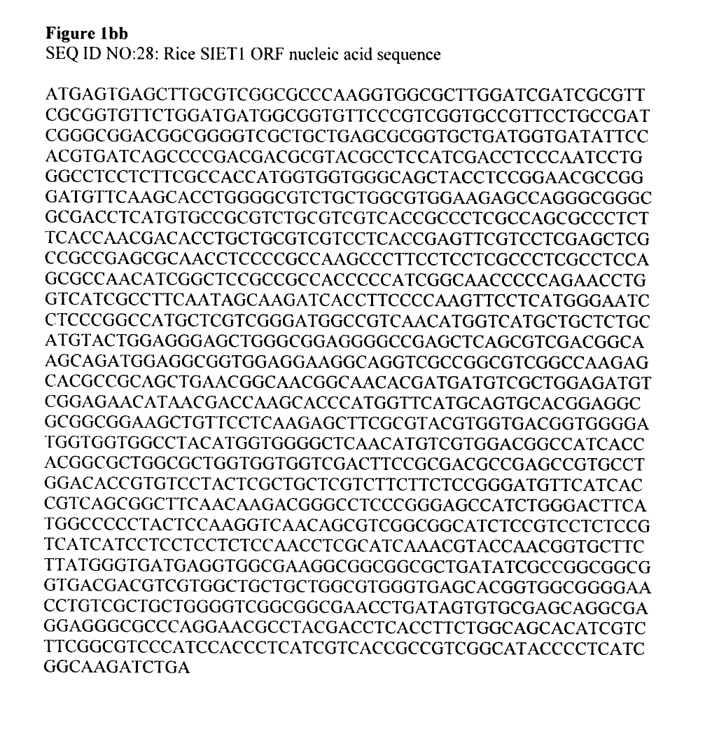

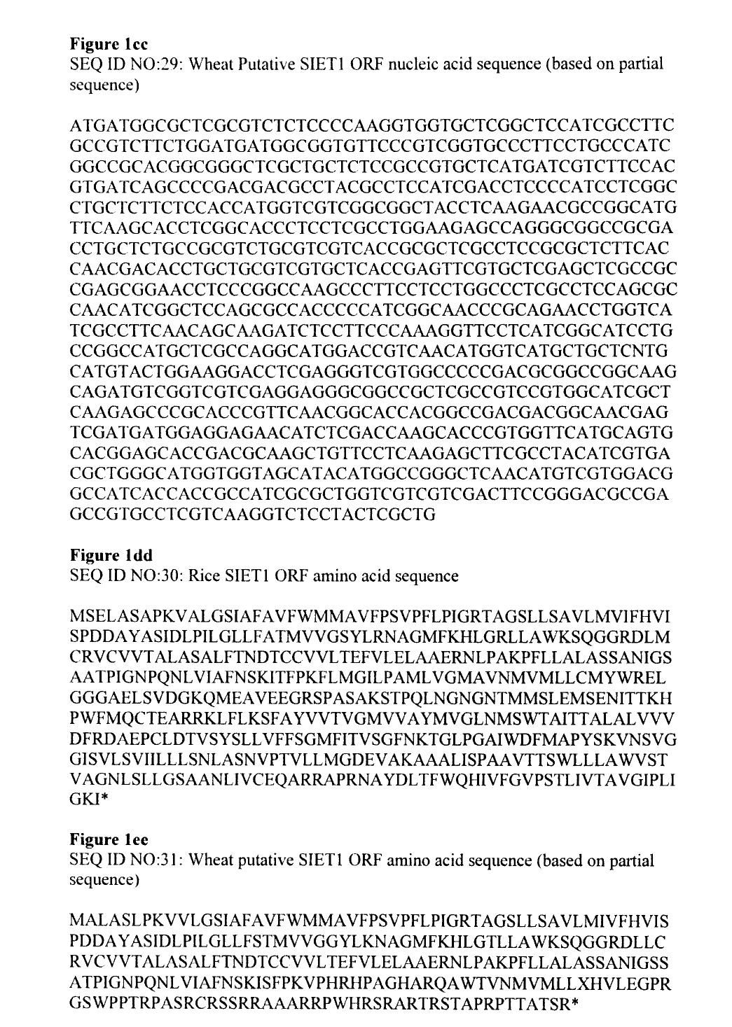

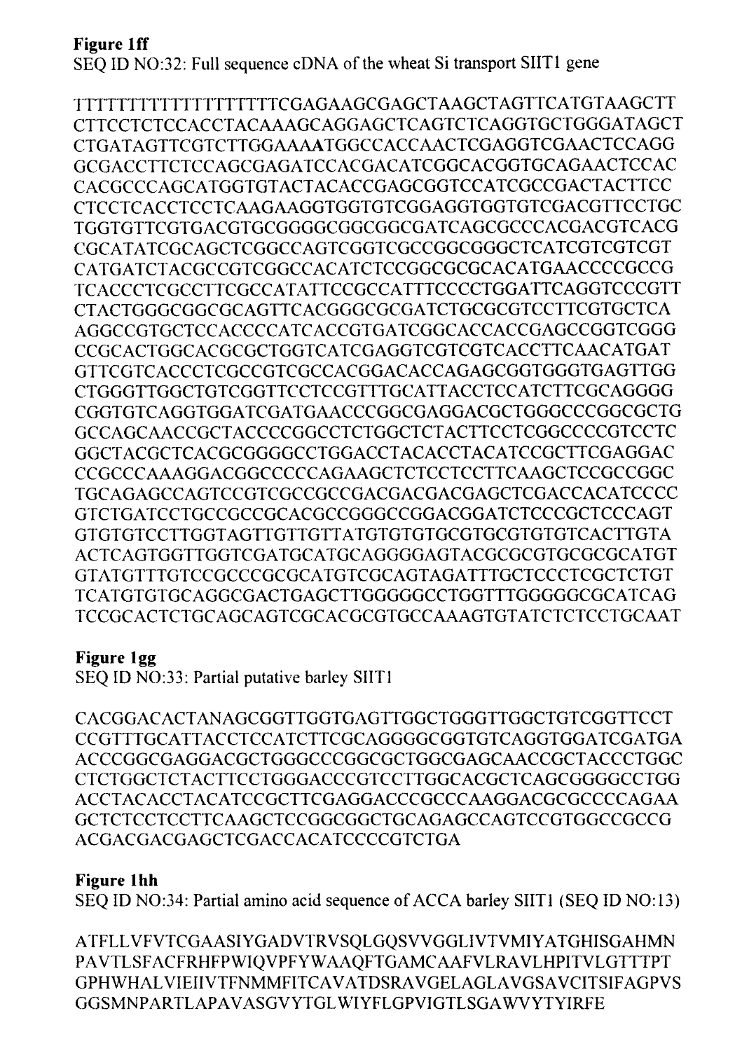

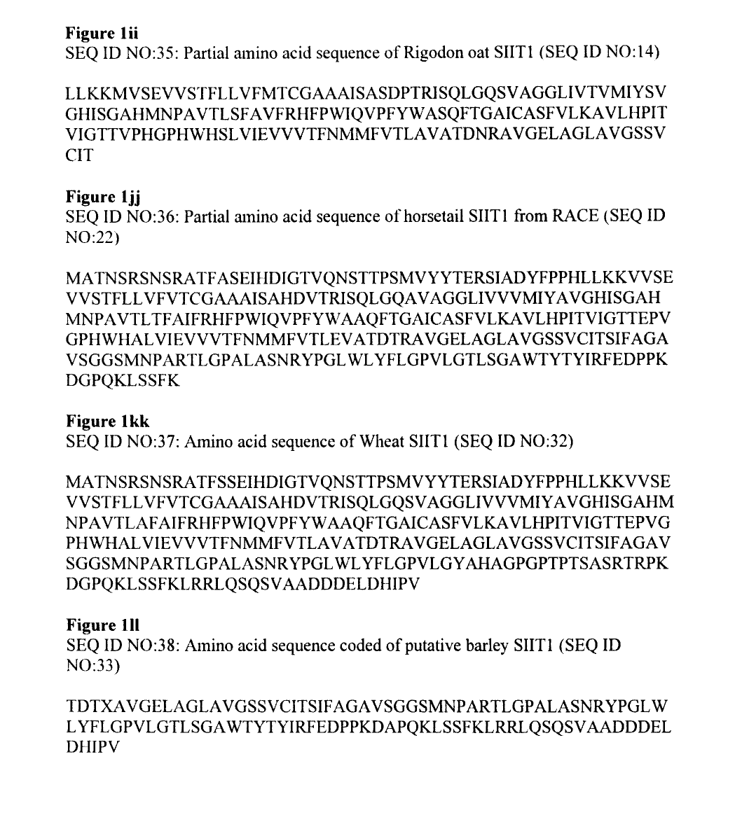

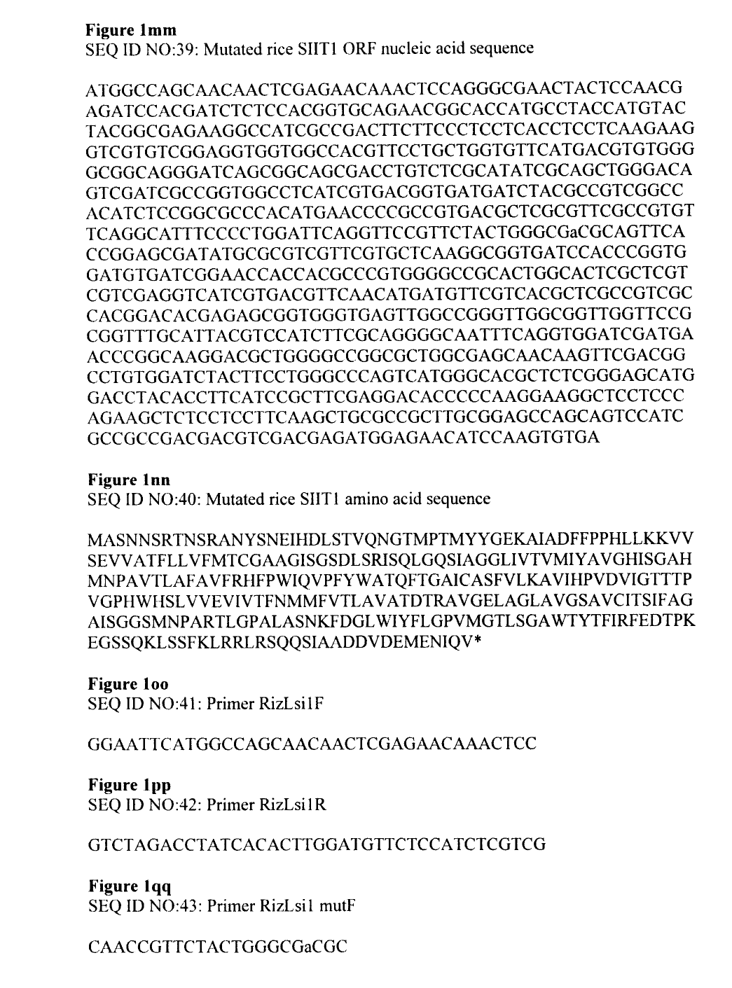

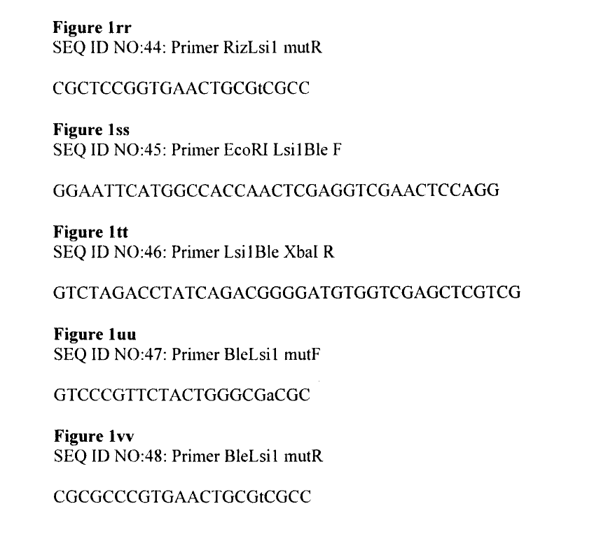

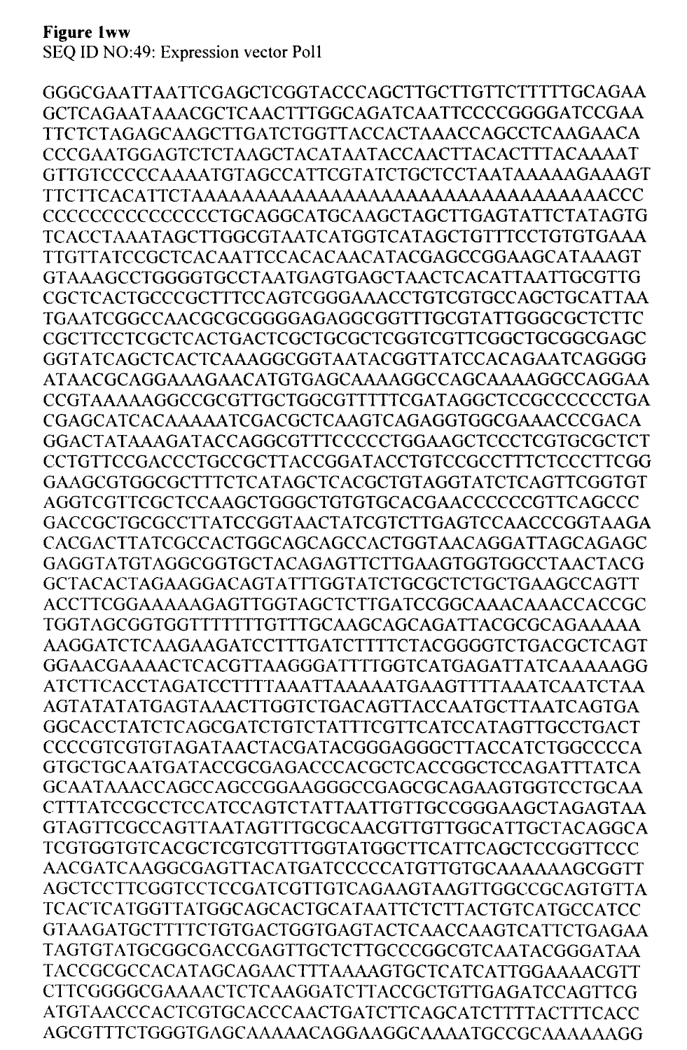

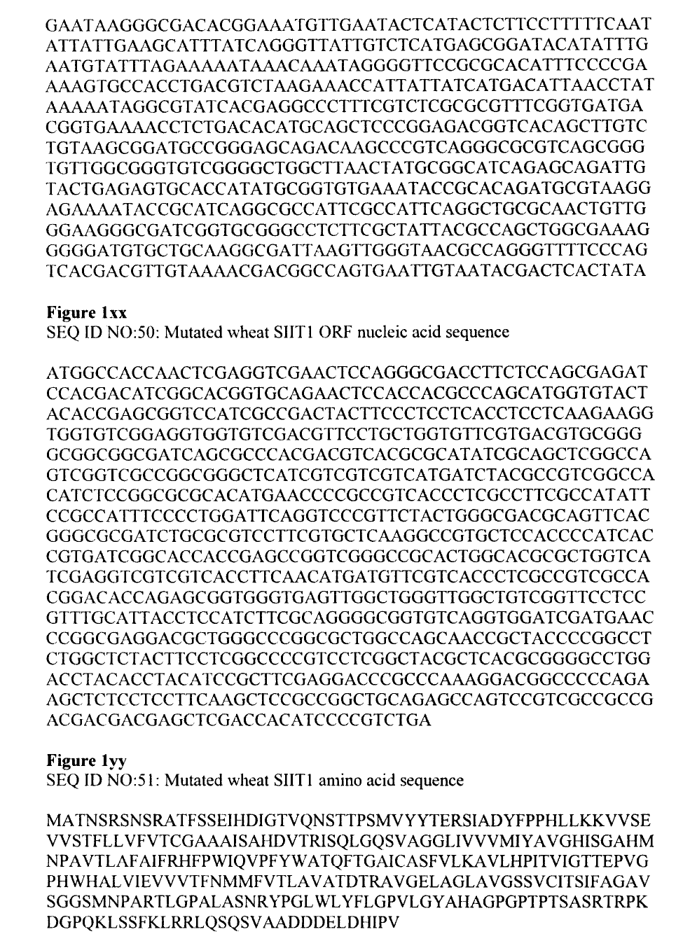

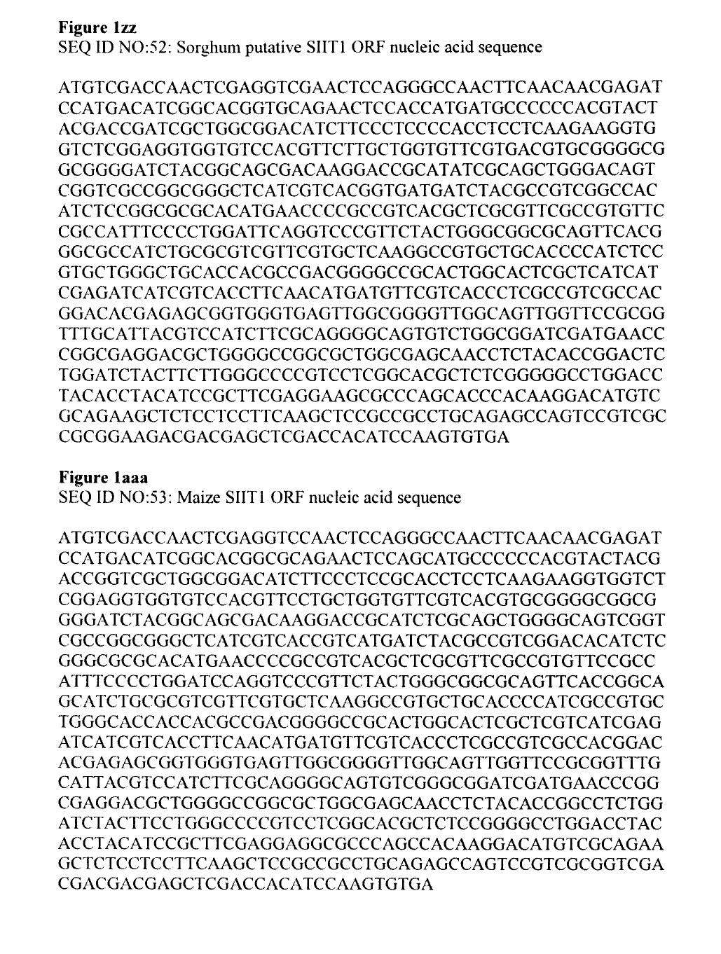

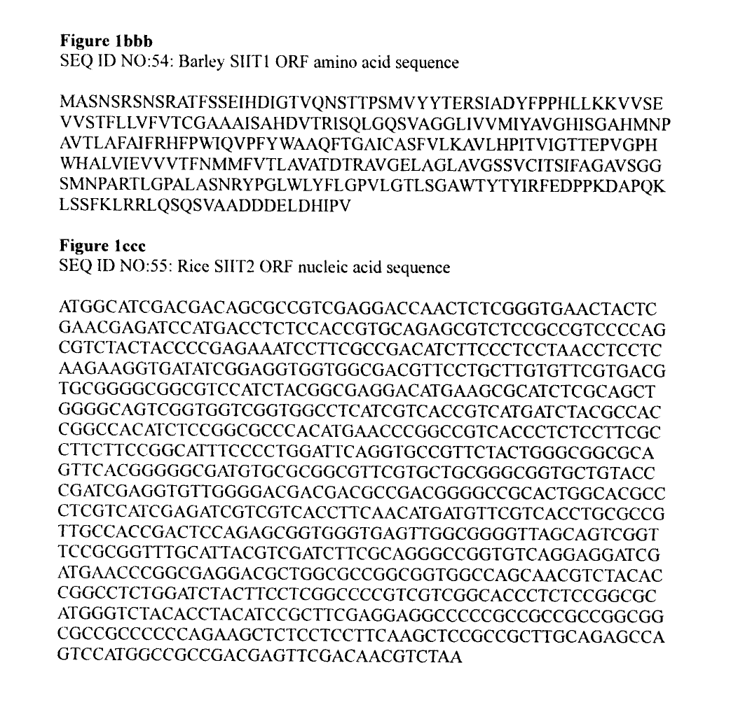

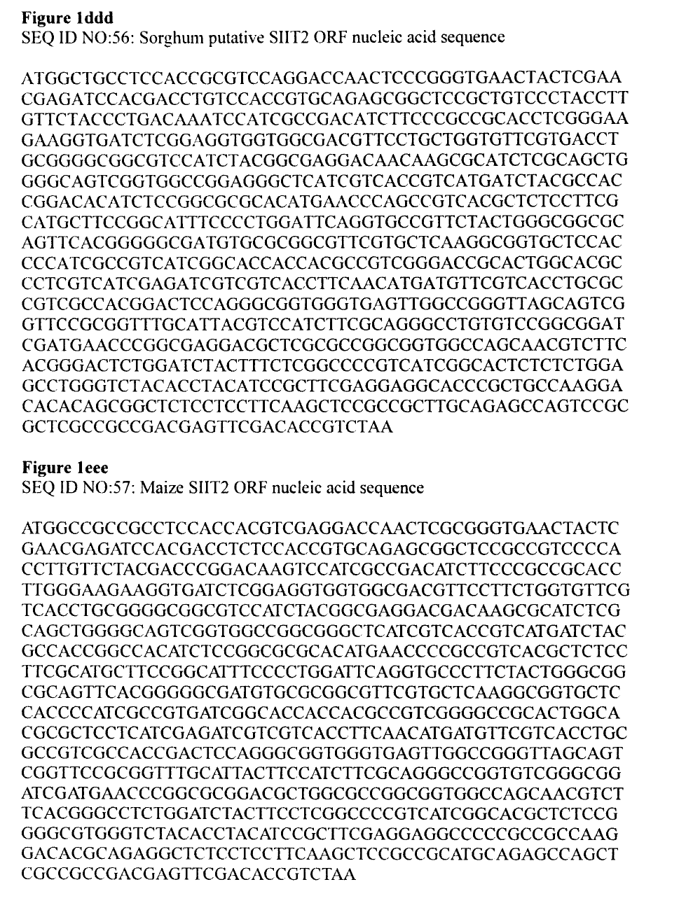

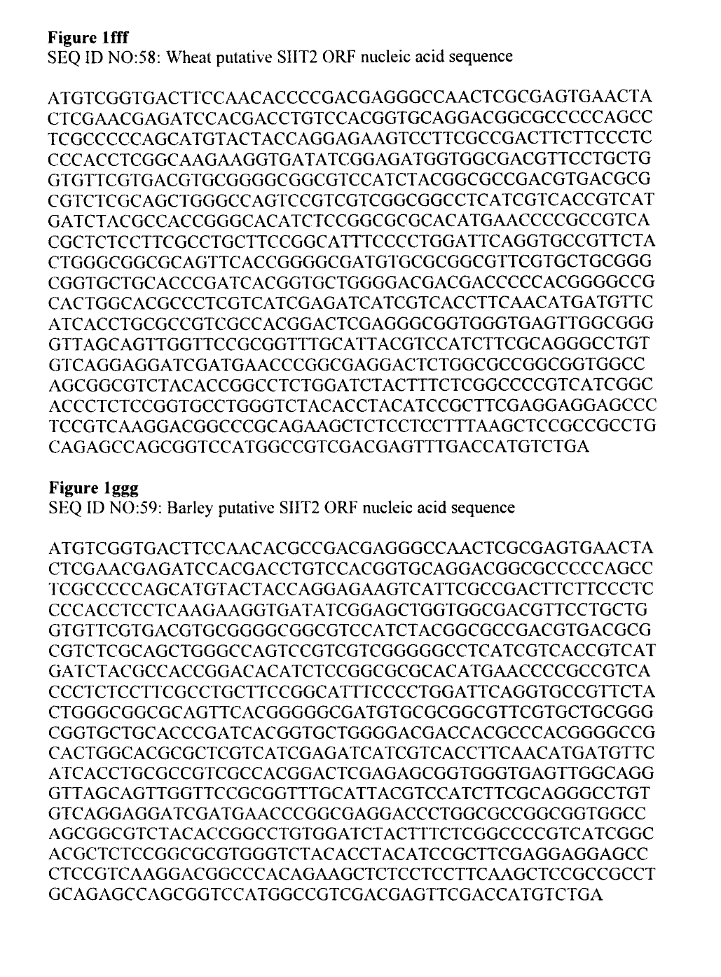

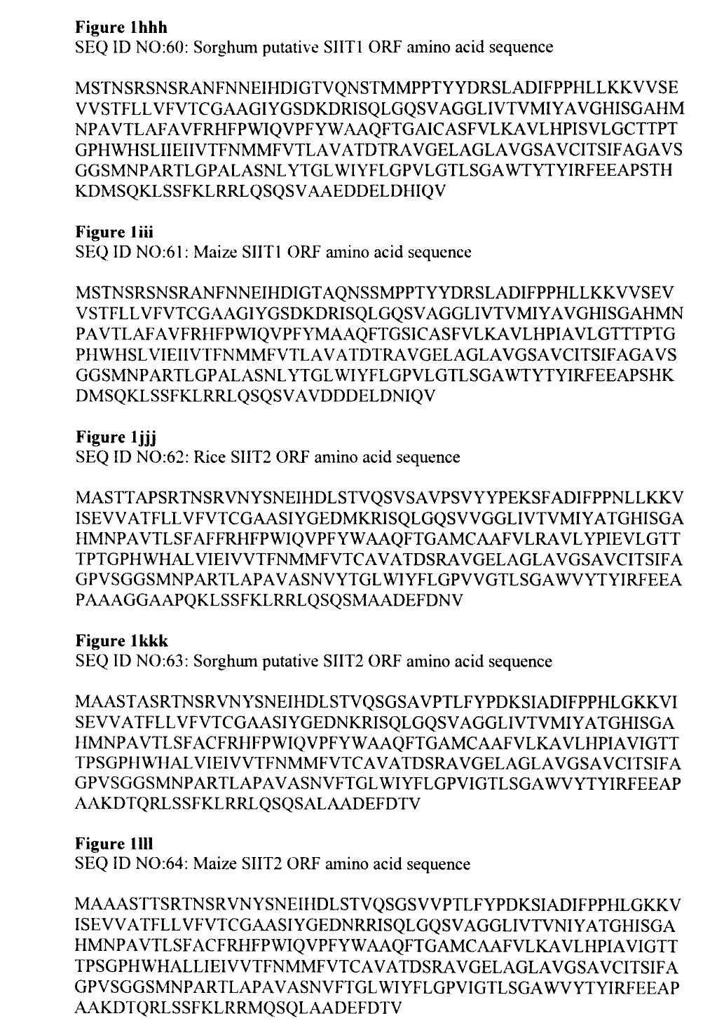

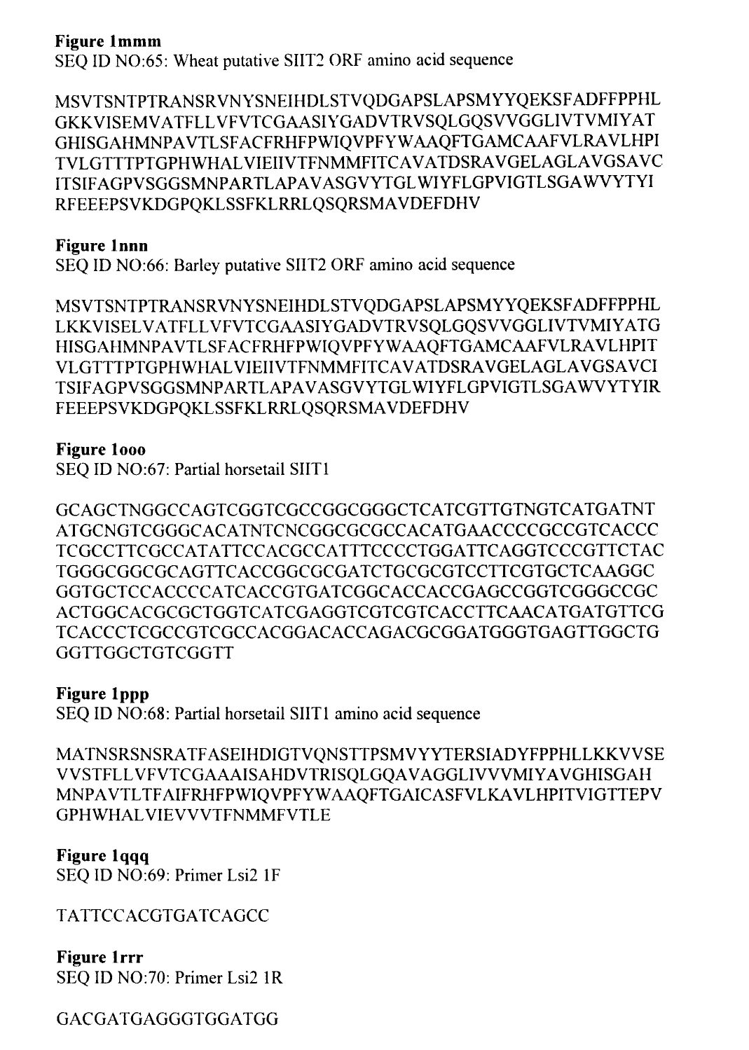

### DETAILED DESCRIPTION

Horsetail and grasses such as wheat, oat, sorghum, and barley are known to be high accumulators of silicon. Horsetail, in particular, is known to accumulate silicon very efficiently, and silicon compounds can make up to 15% of horsetail dry weight. We therefore hypothesized that these plants, due to their high silicon content, would likely transport silicon efficiently, i.e., able to cause accumulation of high concentrations of silicon in the plant, able to transport silicon rapidly, or both. These transporters may therefore be used to increase silicon uptake in a heterologous cell (e.g., in a plant that normally has lower silicon uptake or transport) by expressing a transporter described herein. Because increased silicon content in plants is associated with increased resistance to both biotic and abiotic stresses, expression of these transporters in a plant may increase resistance to stress. Such an approach may be particularly useful in soybean, where soybean rust caused by Phakopsora pachyrhizi fungus causes significant damage to the soybean crop. Accordingly, the present invention features polynucleotides and polypeptides having sequence identity to the silicon transporters identified herein, vectors, cells, and plants (e.g., soybean) containing such polynucleotides, and methods for making such plants. Plants expressing silicon transports may exhibit increased resistance to fungus such as rust.

### Silicon in Plants

Silicon (Si) is absorbed by the root system in the form of silicic acid where it can eventually accumulate in the form of polymerized silicon in the shoots and leaves of plants. However, plants vary greatly in their ability to absorb silicon, thereby causing variability in their ability to benefit from Si feeding. In a survey of nearly 500 plant species, plants were ranked into three groups according to their Si accumulation: 1) high Si accumulators including Gramineae (grasses); 2) intermediate accumulators including Cucurbitaceae; and 3) low accumulators including most other plant species (for a summary see Ma and Takahashi, Soil, Fertilizer, and Plant Silicon Research in Japan, Amsterdam:Elsevier Science, 2002). For example, grasses such as oat, rye, and ryegrass, contained 2.04, 2.41, and 2.34% SiO₂, when grown in soil containing 45 ppm SiO₂ in solution at pH 6.0. By contrast, crimson clover, peas, and mustard, in the same soil, contained 0.12, 0.25, and 0.15% SiO₂, respectively (Jones et al, Advances in Agronomy, 107-149, 1967). Differences in Si accumulation have been attributed to the ability of the roots to take up Si whereby plants would possess one of three modes of absorption: active, passive, or rejective uptake.

Silicon is one of the most abundant elements on the surface of the earth, but its essentiality in plant growth has not been clearly established (Epstein, Silicon in Agriculture. Datnoff et al., eds. New York: Elsevier Science; 2001:1-15; Epstein, Proc Natl Acad Sci USA 91:11-17, 1994; Epstein, Annu Rev Plant Physiol Plant Mol Biol 50:641-664, 1999). While its nutritional role in plants appears limited, there is accumulating evidence that Si absorption plays an important function in protection against biotic and abiotic stresses. Many reports have implicated Si with improved plant growth in situations of nutrient deficiency or excess. Si fertilization has also been linked to increased resistance of plants to diseases, including powdery mildew pathogens on wheat, barley, rose, cucumber, muskmelon, zucchini squash, grape, and dandelion and for other diseases such as blast (Pyricularia grisea) and brown spot (Bipolaris oryzae) on rice, Botrytis cinerea, Didymella bryoniae, Fusarium wilt, and root rot caused by Pythium ultimum and P. aphanidermatum on cucumber.

Three silicon transporters have been identified in rice (Ma et al., Nature 440:688-691, 2006; Ma et al., Nature 448:209-212, 2007; Yamaji et al, The Plant Cell 20: 1381-1389, 2008), including two Si influx transporters (SIIT1, also referred to as Lsi1; SEQ ID NOS:3 and 5, and SIIT2, also referred to as Lsi6; SEQ ID NOS:55 and 62) and a Si efflux transporter (SIET1, also referred to as Lsi2; SEQ ID NOS:28 and 30). The influx transporters SIIT1 and SIIT2 are predicted to be membrane proteins similar to water channel proteins, aquaporins. These proteins belong to the NIP subfamily (Nod26-like major intrinsic protein). The channel is formed from six transmembrane segments (TM), two hydrophilic loops (HL3 between TM3 and TM4; HL4 between TM4 and TM5) and two Asn-Pro-Ala (NPA) motifs, an arrangement that is conserved in aquaporins. A pore structure and constrictions that may determine selective water permeability are assembled with HL3 and the second NPA domain (NPA2) in the extracellular side and with HL4 and the first NPA domain (NPA1) in the cytoplasmic membrane. The NPA boxes may be important for correct assembly of the three-dimensional structures of aquaporins, because such proteins with mutations near NPA boxes can be folded improperly. The expression of the SIIT1 transporter appears to be localized in roots with a constitutive expression regulated by Si level. The transporter SIIT2 appears to be expressed in the root tips and in the xylem parenchyma cells of leaf sheaths and blades.

The rice Si efflux gene, which is predicted to encode a membrane anion transporter with 11 transmembrane domains, has no similarity to the Si influx transporter SIIT1. SIET1 is an active efflux transporter. SIET1 expression in roots appears to follow the same pattern of SIIT1, but is localized on the proximal side of the exodermal and endodermal cells, whereas SIIT1 is localized on the distal side of root cells. SIIT2 also shows polar localization in xylem parenchyma cells on the side facing the xylem vessel. SIIT2 is thought to be involved in transporting Si out of the xylem and into the leaves.

### Polynucleotides

We have identified and cloned silicon transporters from wheat, oat, barley, sorghum, and horsetail. Accordingly, the invention features polynucleotides having substantial identity to any of the polynucleotides described herein, or fragments of such polynucleotides. In certain embodiments, the polynucleotides may encode functional silicon transporter polypeptides (e.g., polypeptides, that when expressed in a cell are capable of increasing silicon influx or efflux). Identification of exemplary polynucleotides of the invention is described in greater detail below.

The invention also features fragments of the polynucleotides described herein. Such fragments may also encode functional silicon transporter polypeptides. Shorter fragments may be useful as primers, or may encode antigenic polypeptide sequences. Fragments may include the transmembrane segments, or the hydrophilic loops of the transporter.

Identification of Silicon Influx Transporters in Plants

We identified silicon transporter sequences in wheat, oat, barley, and horsetail through BLAST searches. Using wheat EST databases, we identified a transporter sequence in wheat and termed this sequence the SIIT1 wheat Si-transport gene (SEQ ID NO:2). Comparison of the wheat cDNA, coding sequences (SEQ ID NO:4), and the 296 amino acid wheat polypeptide sequence (SEQ ID NO:6) to the corresponding rice sequences (SEQ ID NO:3 and SEQ ID NO:5) revealed 70%, 84.2%, and 82% identity, respectively.

We then cloned an SIIT1 gene from wheat plants cv. HY644 recovered from hydroponic culture. Roots were frozen in liquid nitrogen, crushed using a mortar and total RNA was extracted using an RNA purification Kit from QIAGen and stored at −80° C. Total cDNA were prepared using reverse transcriptase (Superscript III, Invitrogen) with oligodT primers.

Primers 1F (TCCCTCCTCACCTCCTCAAGAAG (SEQ ID NO:7)) and 2R (AGCTTGAAGGAGGAGAGCTTCTG (SEQ ID NO:8)) used to verify the presence of the transport gene by PCR in wheat cDNA preparation. PCR was performed at 94° C. for 120 seconds; followed by 30 cycles of 94° C. for 30 seconds; 62° C. for 30 seconds; and 72° C. 90 seconds; followed by 72° C. 10 min. 100 ng of wheat cDNA was used with 0.2 μM of each primer. This PCR reaction amplified a 700 bp fragment, which was then sequenced (SEQ ID NO:9) and compared with the databank EST sequence. Sequence analysis of SEQ ID NO:9 using the ClustalW program indicated a 98.9% identity over the 659 bp overlapping sequence. These differences can likely be explained by the kind of cultivar used to obtain SEQ ID NO:4 in the database and the cultivar used in our experiment.

We then designed two additional reverse primers 3R (CGAAGATGGACGTAATGCAAACC (SEQ ID NO:10)) and 1R (CGCCCAGTAGAACGGAACCT (SEQ ID NO:11)) to identify silicon transporter homologues in other plant species. Total cDNA was obtained from plants including Torka wheat cultivar, ACCA barley cultivar, Rigodon oat cultivar, and horsetail. Plants were grown in a growth cabinet in 6 cm plastic pots filled with Promix PGX growth medium (Premier Horticulture, Rivière-du-Loup, Québec, Canada). Following growth, roots were recovered, washed in distilled water to remove all traces of the Promix substrate, frozen in liquid nitrogen, and crushed using a mortar. Total RNA was extracted using an RNA purification kit from QIAGen. Total cDNA was obtained using the Superscript III reverse transcriptase from Invitrogen using an oligodT primer. The obtained cDNAs were kept at −20° C. The presence of a Si-transport gene in Torka wheat cultivar, ACCA barley cultivar, Rigodon oat cultivar and horsetail was detected using the primer pairs shown in Table 1.

TABLE 1
Primers used

| Organism | Forward Primer | Reverse Primer | Sequence obtained |
|---|---|---|---|
| Wheat cv. | SEQ ID NO: 7 | SEQ ID NO: 8 | SEQ ID NO: 12 |
| Torka | | | |
| Barley cv. | SEQ ID NO: 7 | SEQ ID NO: 8 | SEQ ID NO: 13 |
| ACCA | | | |
| Oat cv. | SEQ ID NO: 7 | SEQ ID NO: 10 | SEQ ID NO: 14 |
| Rigodon | | | |
| Horsetail | SEQ ID NO: 16 | SEQ ID NO: 8 | SEQ ID NO: 15 |

PCR was performed using 100 ng of each cDNA preparation with 0.2 μM of each primer. The PCR reaction was performed at 94° C. for 120 seconds; followed by 30 cycles of 94° C. for 30 seconds; 62° C. for 30 seconds; and 72° C. for 60 seconds; followed by 72° C. 10 minutes. Each amplified fragment was then sequenced. These sequences were compared to the rice and wheat coding sequences (SEQ ID NOS:3 and 4). The corresponding regions of each fragment to the coding sequence and percent identities are shown in Table 2.

TABLE 2
Comparison of fragments to rice and wheat transporters

| | Corresponding | | Corresponding | |
|---|---|---|---|---|
| | region of rice | | region to wheat | |
| | SIIT1 coding | | SIIT1 coding | |
| | sequence (SEQ | Percent | sequence (SEQ | Percent |
| Organism | ID NO: 3) | identity | ID NO: 4) | identity |
| Wheat cv. Torka | 128-800 | 86.3% | 128-800 | 98.8% |
| (SEQ ID NO: 12) | | | | |
| Barley cv. ACCA | 168-782 | 84.0% | 168-782 | 86.3% |
| (SEQ ID NO: 13) | | | | |
| Oat cv. Rigodon | 137-618 | 86.3% | 137-618 | 89.6% |
| (SEQ ID NO: 14) | | | | |
| Horsetail | 1-827 | 84.8% | 1-824 | 98.2% |
| (SEQ ID NO: 15) | | | | |

The amino acid sequence encoded by the above barley and oat polynucleotide sequences are provided in SEQ ID NOS:34 and 35, respectively. The partial barley amino acid sequence (SEQ ID NO:34) corresponds to amino acids 57-260 of the rice and wheat amino acid SIIT1 sequences (SEQ ID NOS:5 and 6). The partial oat amino acid sequence (SEQ ID NO:35) corresponds to amino acids 47-206 of the rice and wheat SIIT1 amino acid sequences.

To obtain the 5′ cDNA end of the SIIT1 Si-transport genes in wheat and horsetail, a RACE (Rapid Amplification of cDNA Ends) procedure was employed. Two forward primers were designed for both wheat and horsetail 3′ RACE (ADApT: GGAATCAGTCAGTAATTGGAGG (SEQ ID NO:16) and ADA: GGAATCAGTCAGTAATTGGAGG (SEQ ID NO:17)). Plant specific primers were designed for reverse primers. For wheat, BleR (TCCTCGAAGCGGATGTAG (SEQ ID NO:18)) and BleRNested (CCTGCGAAGATGGAGGTAA (SEQ ID NO:19)) were used. For horsetail, PreleRNested (CGAGGGTGACGAACATCAT (SEQ ID NO:20)) was used.

A 3′ RACE was performed in wheat. PCR was performed with 100 ng of total wheat cDNA, obtained from oligodT reverse transcription, with 0.2 μM of the ADApT and BleR primers. The product of this amplification was purified using a PCR purification Kit from QIAgen then diluted 100 times. A second PCR was performed with 0.2 μM of ADA and BleRNested primers. Each PCR was performed as follows: 94° C. for 120 seconds; followed by 40 cycles of 94° C. for 60 seconds, 52° C. for 30 seconds, and 72° C. for 60 seconds; followed by 72° C. for 10 min. The amplified fragment was then inserted in pGEM-T vector (Promega) and used to transform an E. coli DH5α strain. The presence of the proper insert was screened using PCR with 0.2 μM of each of M13 forward (M13F) and reverse (M13R) primers under the following conditions: 94° C. for 120 seconds; followed 25 cycles of 94° C. for 30 seconds, 55° C. for 30 seconds, and 72° C. 90 seconds, followed by 72° C. for 5 min. A plasmid extraction was performed on positive clones using a plasmid extraction kit from QIAgen. The insert was sequenced with M13F and M13R primers; the resulting sequence is shown in SEQ ID NO:21. The 5′ region of an ORF corresponding to the wheat coding sequence previously identified (SEQ ID NO:4) is found in SEQ ID NO:21 starting at nucleotide 124 to the end (nucleotide 919). The first 246 amino acids coded for by this sequence are identical to the database sequence of wheat identified in SEQ ID NO:6.

Using the wheat transporter sequence, an additional BLAST search was performed using the following databases: GenBank (National Institutes of Health, Bethesda, Md.), GrainGenes (U.S. Dept. of Agriculture, Washington, D.C.), TIGR wheat genome database (The Institute for Genomic Research, now part of the J. Craig Venter Institute, Rockville, Md.) and BarleyBase (Iowa State University, Ames, Iowa). Based on this sequence, another SIIT1 full sequence cDNA was identified in wheat (SEQ ID NO:32). An open reading frame (ORF) from nucleotides 124 to 1014 is present. This ORF has 98.7% identity to the wheat sequence identified in SEQ ID NO:4. The amino acid sequence encoded by this ORF is shown in SEQ ID NO:37.

We also identified a partial cDNA barley SIIT1 transporter gene in the database (SEQ ID NO:33) by performing a BLAST search of SEQ ID NO:13 in the GrainGenes database. This search identified the partial cDNA of the barley SIIT1 sequence. The barley cDNA fragment (SEQ ID NO:13) is 83% identical to the partial cDNA barley SIIT1 sequence (SEQ ID NO:33). This sequence is 82% identical to the rice sequence (SEQ ID NO:3) and 97% identical to the wheat sequence (SEQ ID NO:4). The amino acid sequence encoded by this ORF is shown in SEQ ID NO:38.

A similar approach was used to clone the horsetail sequence. Total cDNA was obtained from horsetail following the same protocol as described above. Using 100 ng of total horsetail cDNA with 0.2 μM of ADApT and PreleRNested. The product of this amplification was diluted 100 times and a second PCR was performed with 0.2 μM of ADA and PreleRNested. The amplified fragment (700 bp) was purified with the PCR purification kit from QIAgen then inserted in pGEM-T vector (Promega). This vector was used to transform an E. coli DH5α strain. The presence of the proper insert was screened by PCR with 0.2 μM of each of M13 forward (M13F) and reverse (M13R) primers under the following conditions: 94° C. for 120 seconds; followed 25 cycles of 94° C. for 30 seconds, 55° C. for 30 seconds, and 72° C. 90 seconds, followed by 72° C. for 5 min. A plasmid extraction was made on positive clones with the plasmid extraction kit from QIAgen. Plasmids were sequenced with M13F and M13R primers; the resulting sequence is shown in SEQ ID NO:22. This sequence was then compared to the wheat SIIT1 gene. Sequence analysis using the ClustalW program on the 728 overlapping nucleotide residues revealed 97.3% homology between wheat SIIT Si-transport gene (SEQ ID NO:2) and horsetail (SEQ ID NO:22) fragment of SIIT Si-transport gene. We also identified nucleotides 146-694 of the horsetail sequence as corresponding to the nucleotides 1-549 of the rice and wheat coding sequences (SEQ ID NOS:3 and 4). The partial horsetail SIIT1 amino acid is 97% identical to the corresponding amino acids of the wheat SIIT1 sequence and 79% identical to corresponding amino acids of the rice SIIT1 sequence. Tables 3 and 4 show nucleic acid and amino acid identities, respectively, between the various SIIT1 sequences identified.

TABLE 3
Percent identity of SIIT1 silicon transporter nucleic
acid sequences among different plant species.

| | | SIIT1 | | | | |
|---|---|---|---|---|---|---|
| | | rice | sorghum | maize | wheat | barley |
| | | SEQ ID | SEQ ID | SEQ ID | SEQ ID | SEQ ID |
| % ID a.n. | | NO: 3 | NO: 52 | NO: 53 | NO: 4 | NO: 33 |
| SIIT2 | rice SEQ ID NO: 55 | 75.0 | | | | |
| | sorghum SEQ ID NO: 56 | | 78.0 | | | |
| | maize SEQ ID NO: 57 | | | 77.7 | | |
| | wheat SEQ ID NO: 58 | | | | 75.2 | |
| | barley SEQ ID NO: 59 | | | | | 74.9 |

TABLE 4
Percent identity of SIIT1 silicon transporter amino acid sequences
among different plant species.

| | | SIIT1 | | | | | |
|---|---|---|---|---|---|---|---|
| | | rice | sorghum | maize | wheat | barley | horsetail |
| | | SEQ ID | SEQ ID | SEQ ID | SEQ ID | SEQ ID | SEQ ID |
| % ID a.a. | | NO: 5 | NO: 60 | NO: 61 | NO: 37 | NO: 54 | NO: 36 |
| SIIT1 | rice SEQ ID NO: 5 | 100 | 81.3 | 81.2 | 81.9 | 82.3 | 82.2 |
| | sorghum SEQ ID NO: 60 | | 100 | 96.0 | 85.9 | 85.2 | 84.1 |
| | maize SEQ ID NO: 61 | | | 100 | 85.2 | 84.5 | 83.7 |
| | wheat SEQ ID NO: 37 | | | | 100 | 98.0 | 98.2 |
| | barley SEQ ID NO: 54 | | | | | 100 | 96.7 |
| | horsetail SEQ ID NO: 36 | | | | | | 100 |

Identification of Additional Si Influx transporters, Including SIIT2 Sequences

We have also identified SIIT2 genes by BLAST analysis in wheat (SEQ ID NO:58), sorghum (SEQ ID NO:56), and barley (SEQ ID NO:59). The wheat and barley SIIT2 sequences were identified in the Gene Index Databases at the Dana-Farber Cancer Institute, Boston, Mass. (available online at http://biocomp.dfci.harvard.edu/tgi/tgipage.html). The sorghum sequence was identified in the PlantGDB Database (available online athttp://www.plantgdb.org/). The barley cDNA sequence identified above (SEQ ID NO:13) is identical to a portion of the barley SIIT2 sequence found in the databank (SEQ ID NO:59). These sequences were compared to the rice (SEQ ID NO:55) and maize (SEQ ID NO:57) sequences, as shown in Table 5. The translated amino acid sequences are shown in Table 6.

TABLE 5
Identity percentage of nucleic acid sequences between
SIIT2 silicon transporters of different plant species.

| | | SIIT2 | | | | |
|---|---|---|---|---|---|---|
| | | rice | sorghum | maize | wheat | barley |
| | | SEQ ID | SEQ ID | SEQ ID | SEQ ID | SEQ ID |
| % ID a.n. | | NO: 55 | NO: 56 | NO: 57 | NO: 58 | NO: 59 |
| SIIT2 | rice SEQ ID NO: 55 | 100 | 88.7 | 88.9 | 88.1 | 88.6 |
| | sorghum SEQ ID NO: 56 | | 100 | 94.1 | 86.6 | 86.0 |
| | maize SEQ ID NO: 57 | | | 100 | 86.3 | 86.6 |
| | wheat SEQ ID NO: 58 | | | | 100 | 97.6 |
| | barley SEQ ID NO: 59 | | | | | 100 |

TABLE 6
Identity percentage of amino acid sequences between SIIT2
silicon transporters of different plant species.

| | | SIIT2 | | | | |
|---|---|---|---|---|---|---|
| | | rice | sorghum | maize | wheat | barley |
| | | SEQ ID | SEQ ID | SEQ ID | SEQ ID | SEQ ID |
| % ID a.a. | | NO: 62 | NO: 63 | NO: 64 | NO: 65 | NO: 66 |
| SIIT2 | rice SEQ ID NO: 62 | 100 | 89.3 | 87.2 | 86.8 | 87.0 |
| | sorghum SEQ ID NO: 63 | | 100 | 96.6 | 84.7 | 84.3 |
| | maize SEQ ID NO: 64 | | | 100 | 84.3 | 84.0 |
| | wheat SEQ ID NO: 65 | | | | 100 | 99.3 |
| | barley SEQ ID NO: 66 | | | | | 100 |

### Comparison of SIIT1 and SIIT2 Sequences

We have compared the SIIT1 and SIIT2 nucleic acid sequences identified above within the same species. This comparison is shown in Table 7.

TABLE 7
Identity percentage between nucleic acid sequences of SIIT1 and
SIIT2 silicon influx transporters of the same plant species.

| | | SIIT1 | | | | |
|---|---|---|---|---|---|---|
| | | rice | sorghum | maize | wheat | barley |
| | | SEQ ID | SEQ ID | SEQ ID | SEQ ID | SEQ ID |
| % ID a.n. | | NO: 3 | NO: 52 | NO: 53 | NO: 4 | NO: 33 |
| SIIT2 | rice SEQ ID NO: 55 | 75.0 | | | | |
| | sorghum SEQ ID NO: 56 | | 78.0 | | | |
| | maize SEQ ID NO: 57 | | | 77.7 | | |
| | wheat SEQ ID NO: 58 | | | | 75.2 | |
| | barley SEQ ID NO: 59 | | | | | 74.9 |

A similar comparison was made between the amino acid sequences of the SIIT1 and SIIT2 sequences, as shown in Table 8.

TABLE 8
Identity percentage between amino acid sequences of SIIT1 and
SIIT2 silicon influx transporters of the same plant species

| | | SIIT1 | | | |
|---|---|---|---|---|---|
| | | rice | sorghum | maize | wheat |
| | | SEQ ID | SEQ ID | SEQ ID | SEQ ID |
| % ID a.a. | | NO: 5 | NO: 60 | NO: 61 | NO: 37 |
| SIIT2 | rice SEQ ID NO: 62 | 78.0 | | | |
| | sorghum SEQ ID NO: 63 | | 81.2 | | |
| | maize SEQ ID NO: 64 | | | 82.3 | |
| | wheat SEQ ID NO: 65 | | | | 79.7 |

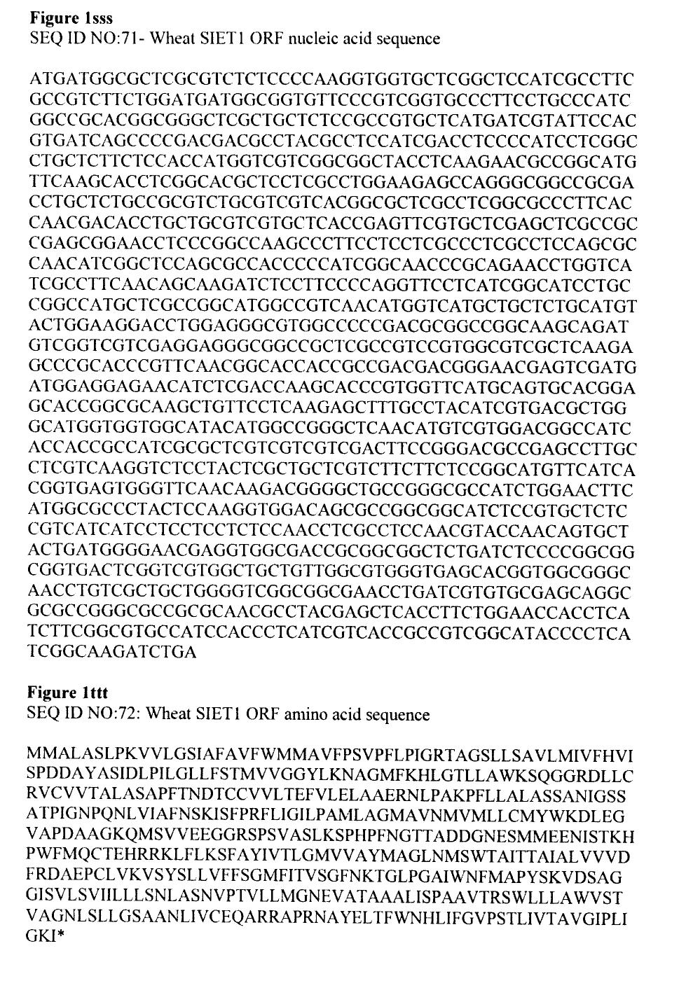

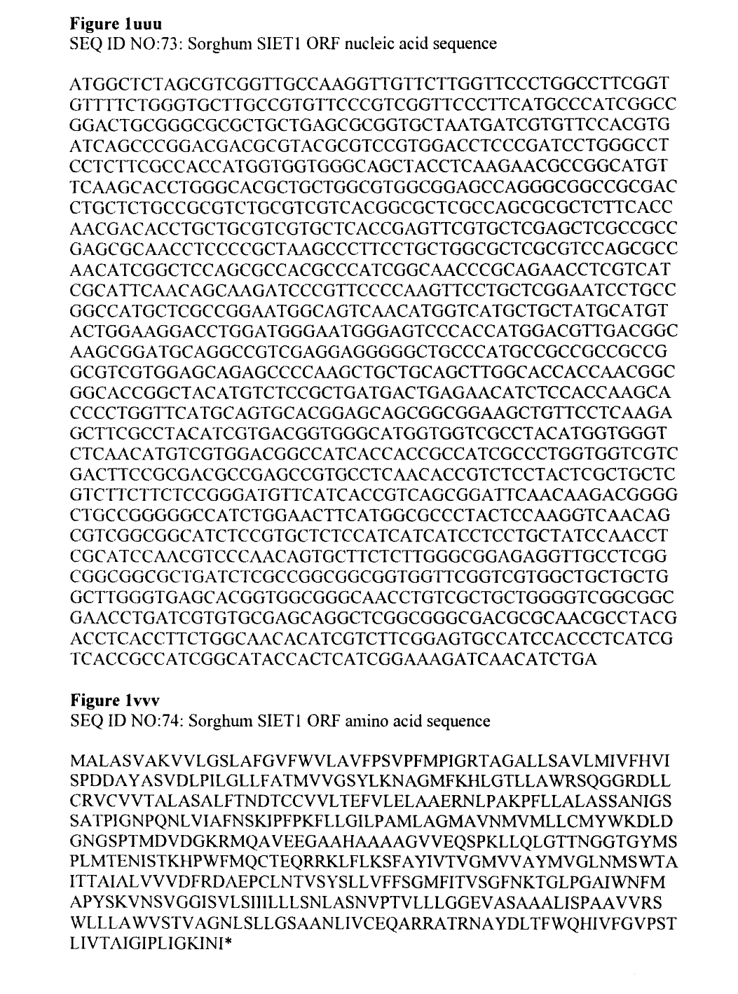

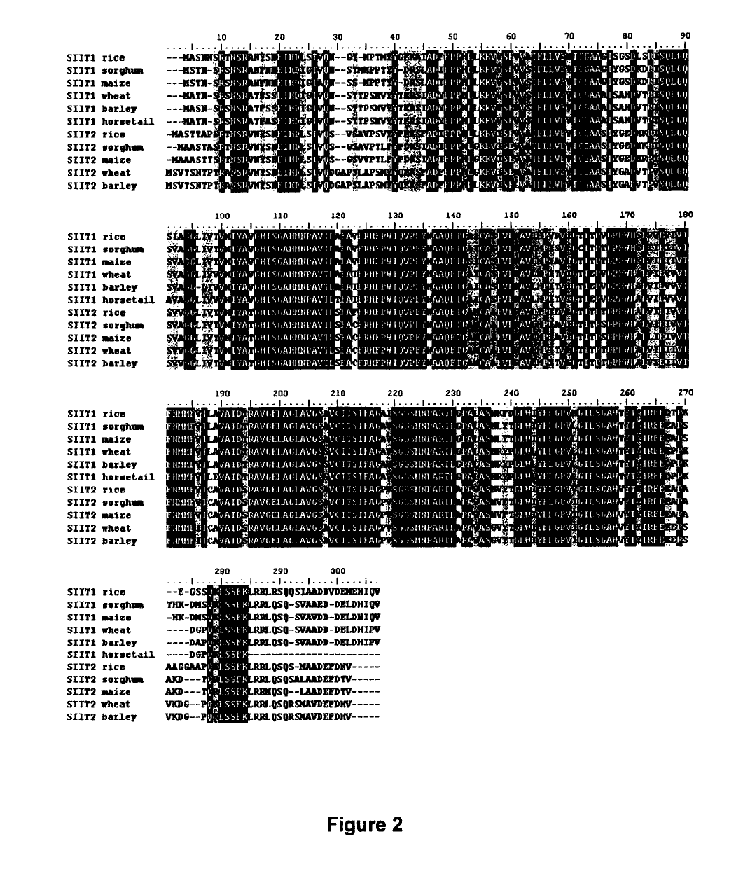

Finally, we have generated a sequence alignment among the SIIT1 and SIIT2 sequences, as shown in FIG. 2.

### Identification of Silicon Efflux Transporters (SIET1) in Plants

We have also identified an SIET1 gene in wheat (SEQ ID NO:24) by BLAST searching and analysis using the ClustalW program. We then designed new primers (Lsi2 F56 (SEQ ID NO:25) and Lsi2 R100 (SEQ ID NO:26)) to check for the presence of a putative SIET1 wheat Si-transport gene in our cultivar. PCR was therefore performed with 0.2 μM each of the Lsi2 F56 and Lsi2 R100 primers with 100 ng of total wheat cDNA obtained as described before. The PCR was performed as follows: 94° C. for 120 seconds; followed by 35 cycles of 94° C. for 30 seconds, 56° C. for 30 seconds, and 72° C. for 90 seconds; followed by 72° C. for 5 min. The amplified fragment (850 bp) was purified (PCR purification kit from QIAgen), inserted in a pGEM-T-T vector (Promega), which was used to transform an E. coli DH5α, strain. Presence of the proper insert was determined by PCR using the M13F and M13R primers, as described above. A plasmid extraction was made on positive clones (plasmid extraction kit from QIAgen). The plasmids were then sequenced using the M13F and M13R primers; the resulting sequence is shown in SEQ ID NO:27.

The cloned wheat (SEQ ID NO:27) SIET1 sequence have 83.5% identity within the 850 bp of the overlapping region with the rice sequence (SEQ ID NO:23). Comparison of the rice SIET1 (SEQ ID NO:23) and the wheat SIET1 database sequence (SEQ ID NO:24) indicated 76.5% identity over the same region. Finally, the wheat SIET1 gene (SEQ ID NO:24) and the sequenced fragment obtained in our study (SEQ ID NO:27) are 98.5% identical. These differences are likely due to natural genetic variations between different wheat cultivars.

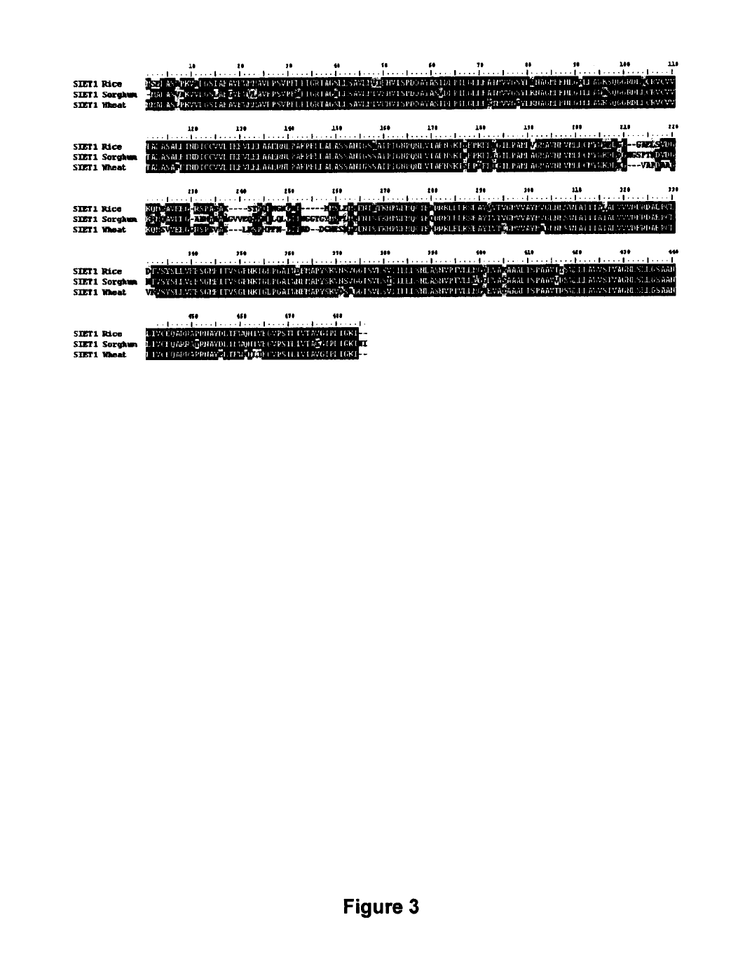

The deduced open reading frames (SEQ ID NOS:28 and 29, respectively, which correspond to nucleotides 152-1570 of SEQ ID NO:23 and nucleotides 220-981 of SEQ ID NO:24) of the rice and wheat SIET1 genes are shown, as are the encoded amino acid sequences (SEQ ID NOS:30 and 31, respectively). SEQ ID NOS:28 and 29 are 57% identical and SEQ ID NOS:30 and 31 are 38.9% identical. New primers (SEQ ID NO:69; SEQ ID NO:70) were designed after comparison with other SIET1 sequences to amplify the complete wheat SIET1 ORF. The resulting sequences are shown in SEQ ID NO:71 and SEQ ID NO:72. A comparison between SIET1 sequences from rice, sorghum (PlantGDB Database) and wheat is represented in Table 9 (nucleic acid sequence) and 10 (amino acid sequence). An alignment of SIET1 amino acid sequence is shown in FIG. 3.

TABLE 9
Identity percentage of nucleic acid sequence between
SIET1 silicon transporters of different plant species

| | | SIET1 | | |
|---|---|---|---|---|
| | | rice | sorghum | wheat |
| | | SEQ ID | SEQ ID | SEQ ID |
| % ID | | NO: 28 | NO: 73 | NO: 71 |
| SIET1 | rice SEQ ID NO: 28 | 100 | 83.8 | 85.4 |
| | sorghum SEQ ID NO: 73 | | 100 | 85.5 |
| | wheat SEQ ID NO: 71 | | | 100 |

TABLE 10
Identity percentage of amino acid sequence between SIET1
silicon transporters of different plant species

| | | SIET1 | | |
|---|---|---|---|---|
| | | rice | sorghum | wheat |
| | | SEQ ID | SEQ ID | SEQ ID |
| % ID | | NO: 30 | NO: 74 | NO: 72 |
| SIET1 | rice SEQ ID NO: 30 | 100 | 84.3 | 86.1 |
| | sorghum SEQ ID NO: 74 | | 100 | 84.1 |
| | wheat SEQ ID NO: 72 | | | 100 |

### Characterization of Si Transporters—Oocyte Assay

To assess and compare the efficiency of Si-transporters encoded by the polynucleotides described herein, transformed oocytes from Xenopus laevis can be used. Si-transporter cRNA can be generated using any method known in the art and can be injected into the oocytes, resulting in production of functional Si-transport proteins. Using this system, the rate of silicon uptake or efflux for different transporters can be evaluated. Such a system allows the characterization of Si-transporter(s) and selection of transporters with desirable traits, including more rapid rate of silicon uptake or a greater total silicon uptake. Alternatively, silicon efflux transporters can be evaluated for their ability to remove silicon from oocytes.

Oocytes have been widely used to study proteins through transient overexpression of the corresponding genes. Ooctyes are particularly well suited for studies of receptors, channels, and ion pumps because these proteins often display normal electrophysiological characteristics in oocytes. It is therefore possible to study assembly, membrane insertion, and function of such proteins. In addition, because oocytes are mammalian cells, complex proteins that require post-translational modification can be produced and retain their functionality.

As noted above, such oocytes can be injected with cRNA to produce transient production of the encoded protein. A gene of interest can be cloned into an expression vector capable of producing cRNA containing the gene. The production of a functional cRNA can be obtained by in vitro transcription of the DNA sequence of the gene of interest to produce a pre-cRNA. The pre-cRNA is then capped with a 7-methylguanosine, which mimics most eukaryotic mRNAs found in vivo. Capping of RNA improves its stability and therefore the yield of translation. Purified, capped cRNA can then be microinjected in prepared oocytes. Such a process is described by Hildebrand et al. (Nature 385:688-689, 1997) to study a silicon transporter found in diatoms. Following oocyte transformation, the rate of silicon influx or efflux can be measured using any method known in the art (e.g., those described herein). Exemplary approaches are also described in Ma et al. (Nature 440:688-691, 2006). Ma et al. use germanium 68 (⁶⁸Ge) as a tracer for silicon to assay uptake into Xenopus oocytes. Because germanium is toxic, it is used at relatively low concentrations in assays where viability of the cell is required. By measuring the radioactivity of low germanium concentrations in the oocytes in the presence or absence of a putative silicon transporter, it is possible to determine whether expression of a particular protein lead to increased silicon transport. To determine whether silicon is specifically transported upon expression of the protein, transport of a molecule such as glycerol can be used as a negative control.

An analogous assay can be used to measure silicon efflux from oocytes. In this approach, oocytes are preloaded with ⁶⁸Ge, and then injected with a test RNA encoding a putative silicon efflux transporter. Following expression of the RNA, extracellular ⁶⁸Ge is measured. An increase in transport of ⁶⁸Ge upon RNA injection is thus indicative of silicon efflux activity.

### Construction of Mutant Silicon Transporters

To assess the feature of Si-transporters encoded by the polynucleotides described herein, rice and wheat SIIT1 mutants were generated by modification of the nucleic acid sequence. Ma et al. (Plant Physiol. 130: 2111-2117, 2002) identified a defective rice silicon transporter characterized by a single point mutation at position 394 in the ORF nucleic acid sequence (SEQ ID NO:39) where a guanine was replaced by an adenosine. This mutation modified the amino acid sequence of the corresponding protein by replacing the alanine by a threonine at position 132 (SEQ ID NO:40) which seemed to be a critical residue because this substitution significantly altered the conformation of the protein and led to a loss in silicon uptake.

We reproduced this mutation in both rice and wheat using an appropriate set of primers. For rice, in a first step, we amplified separately the 5′ and the 3′ end of the nucleic acid sequence (SEQ ID NO:3) using primers RizLsi1F (GGAATTCATGGCCAGCAACAACTCGAGAACAAACTCC (SEQ ID NO:41)) and RizLsi1 mutR (CGCTCCGGTGAACTGCGtCGCC (SEQ ID NO:44)) for the 5′ end and primers RizLsi1 mutF (CAACCGTTCTACTGGGCGaCGC (SEQ ID NO:43)) and RizLsi1R (GTCTAGACCTATCACACTTGGATGTTCTCCATCTCGTCG (SEQ ID NO:42)) for the 3′ end. Primers called “mut” were used to create the point mutation by PCR. A first PCR round was performed at 94° C. for 120 seconds; followed by 35 cycles of 94° C. for 30 seconds; 62° C. for 30 seconds; and 72° C. 90 seconds; followed by 72° C. 10 min. One hundred ng of rice cDNA were used with 0.2 μM of each primer. This PCR reaction amplified a 400 bp fragment for the 5′ half and a 500 bp fragment for the 3′ half of SIIT1 coding sequence. Both halves were purified by a PCR purification kit from QIAgen, quantified, and used for a second round of PCR. The primers RizLsi1F (SEQ ID NO:41) and RizLsi1R (SEQ ID NO:42) were used to obtain the full length mutated coding sequence. The PCR was performed at 94° C. for 120 seconds; followed by 35 cycles of 94° C. for 30 seconds; 62° C. for 30 seconds; and 72° C. 90 seconds; followed by 72° C. 10 min. A mix of 50 ng of each half rice cDNA amplicon was used with 0.2 μM of each primer. The ligation of both half coding sequences was confirmed by a 1.5% agorose gel electrophoresis for the presence of a ca 900 bp fragment, which was purified and inserted in pGEM-T vector (Promega) following manufacturer's instructions. This plasmid was sequenced to confirm the presence of the mutation (SEQ ID NO:39). The translation gave the amino acid sequence of the mutated protein (SEQ ID NO:40).

As the nucleic acid sequence was very similar around the point mutation in both rice and wheat, we decided to reproduce the same protein for the wheat SIIT1 to verify if we were able to reproduce the results in wheat. A procedure similar to that used to obtain the rice mutant was employed, but primers specific to the wheat sequence (SEQ ID NO:32) were used instead. For the first round of PCR, the primers used were EcoRI Lsi1Ble F (GGAATTCATGGCCACCAACTCGAGGTCGAACTCCAGG (SEQ ID NO:45)), Lsi1Ble XbaI R (GTCTAGACCTATCAGACGGGGATGTGGTCGAGCTCGTCG (SEQ ID NO:46)), BleLsi1 mutF (GTCCCGTTCTACTGGGCGaCGC (SEQ ID NO:47)), and BleLsi1 mutR (CGCGCCCGTGAACTGCGtCGCC (SEQ ID NO:48)). For the second round of PCR, the primers used were EcoRI Lsi1Ble F (SEQ ID NO:45) and Lsi1Ble XbaI R (SEQ ID NO:46). Purified ligated fragments were inserted in pGEM-T vector (Promega) following the manufacturer's instructions. This plasmid was sequenced to verify the presence of the mutation (SEQ ID NO:50). The translation gave the amino acid sequence of the mutated protein (SEQ ID NO:51).

These constructs were transformed in E. coli DH5α strains and kept frozen at −80° C.

### Cloning of Silicon Transporters in an Expression Vector

An expression vector, Pol1 (SEQ ID NO: 49), was used for the in vitro transcription of SIIT1 coding sequences. These sequences were inserted into the Pol1 vector using restriction sites that were added by the primers used for PCR amplification: an EcoRI/XbaI fragment containing the coding sequence of SIIT1 was inserted from pGEMt to Pol1 vector using an excision/ligation procedure known in the art. New vectors were transformed in E. coli DH5α strains and kept frozen at −80° C.

Production of cRNAs for Oocytes Microinjection

Plasmids were recovered from a fresh bacteria culture using a miniprep plasmid extraction kit from QIAgen. Plasmids were digested with Nhe1 restriction enzyme (Roche) allowing the linearization of the plasmid. The digestion was purified using a PCR purification kit (QIAgen) and 1 μg fd DNA was used for the in vitro transcription using the mMessage mMachine T7 Ultra kit (Ambion). cRNA were recovered, solubilized in DEPC-treated water and kept frozen at −80° C. until use.

### Promoters

Any polynucleotide described herein can be operatively linked to an appropriate promoter to confer gene expression (e.g., in a cell or in an in vitro system such as a cell extract). Promoters can regulate expression in a time-dependent, cell specific (e.g., root cells), or tissue specific manner. Promoters useful in the expression cassettes of the invention include any promoter that is capable of initiating transcription in a plant cell. Such promoters include those that can be obtained from plants, plant viruses, and bacteria that contain genes that are expressed in plants, such as Agrobacterium and Rhizobium. In one embodiment, the promoter is constitutively active in root cells (e.g., the At17.1 promoter). In another embodiment, the promoter is induced by a biotic or abiotic stress.

The promoter may be constitutive, inducible, developmental stage-preferred, cell type-preferred, tissue-preferred, or organ-preferred. Constitutive promoters are active under most conditions. Examples of constitutive promoters include the CaMV 19S and 35S promoters (Odell et al., Nature 313:810-812,1985), the sX CaMV 35S promoter (Kay et al., Science 236:1299-1302, 1987), the Sep1 promoter, the rice actin promoter (McElroy et al., Plant Cell 2:163-171, 1990), the Arabidopsis actin promoter, the ubiquitin promoter (Christensen et al., Plant Mol. Biol. 18:675-689, 1989), pEmu (Last et al., Theor. Appl. Genet. 81:581-588, 1991), the figwort mosaic virus 35S promoter, the Smas promoter (Velten et al., EMBO J 3:2723-2730, 1984), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, and the small subunit of ribulose biphosphate carboxylase (ssuRUBISCO) promoter.

In other embodiments, an inducible promoter is used. Such promoters are preferentially active under certain environmental conditions, such as the presence or absence of a nutrient or metabolite, heat or cold, light, pathogen attack, anaerobic conditions, or under any abiotic or biotic stress (e.g., those described herein). For example, the hsp80 promoter from Brassica is induced by heat shock; the PPDK promoter is induced by light; the PR-1 promoters from tobacco, Arabidopsis, and maize are inducible by infection with a pathogen; and the Adh1 promoter is induced by hypoxia and cold stress. Plant gene expression can also be facilitated via an inducible promoter (for review, see Gatz, Annu. Rev. Plant Physiol. Plant Mol. Biol. 48:89-108, 1997). Chemically inducible promoters are especially suitable if gene expression is wanted to occur in a time specific manner. Examples of such promoters are a salicylic acid inducible promoter (PCT Publication No. WO 95/19443), a tetracycline inducible promoter (Gatz et al., Plant J. 2:397-404, 1992), and an ethanol inducible promoter (PCT Publication No. WO 93/21334). An inducible promoter is a stress-inducible promoter. Such promoters may be activated based on sub-optimal conditions associated with salinity, drought, temperature, metal, chemical, pathogenic, and oxidative stresses. Stress inducible promoters include, but are not limited to, Cor78 (Chak et al., Planta 210:875-883, 2000; Hovath et al., Plant Physiol. 103:1047-1053, 1993), Cor15a (Artus et al., Proc Natl Acad Sci USA 93:13404-09, 1996), Rci2A (Medin et al., Plant Physiol. 125:1655-66, 2001; Nylander et al., Plant Mol. Biol. 45:341-52, 2001; Navarre et al., EMBO J. 19:2515-24, 2000; Capel et al., Plant Physiol. 115:569-76, 1997), Rd22 (Xiong et al., Plant Cell 13:2063-83, 2001; Abe et al., Plant Cell 9:1859-68, 1997; Iwasaki et al., Mol. Gen. Genet. 247:391-8, 1995), cDet6 (Lang et al., Plant Mol. Biol. 20:951-62, 1992), ADH1 (Hoeren et al., Genetics 149:479-90, 1998), KAT1 (Nakamura et al., Plant Physiol. 109:371-4, 1995), KST1 (Muller-Rober et al., EMBO 14:2409-16, 1995), Rhal (Terryn et al., Plant Cell 5:1761-9, 1993; Terryn et al., FEBS Lett. 299:287-90, 1992), ARSK1 (Atkinson et al., 1997, GenBank Accession #L22302, and PCT Publication No. WO 97/20057), PtxA (Plesch et al., GenBank Accession #X67427), SbHRGP3 (Ahn et al., Plant Cell 8:1477-90, 1996), GH3 (Liu et al., Plant Cell 6:645-57, 1994), the pathogen inducible PRP1-gene promoter (Ward et al., Plant. Mol. Biol. 22:361-366, 1993), the heat inducible hsp80-promoter from tomato (U.S. Pat. No. 5,187,267), cold inducible α-amylase promoter from potato (PCT Publication No. WO 96/12814), or the wound-inducible pinII-promoter (European Pat. No. 375091). Other examples of drought, cold, and salt-inducible promoters, such as the RD29A promoter, are described by Yamaguchi-Shinozalei et al. (Mol. Gen. Genet. 236:331-340, 1993).

Developmental stage-preferred promoters are preferentially expressed at certain stages of development. Tissue and organ preferred promoters include those that are preferentially expressed in certain tissues or organs, such as roots, xylem, leaves, or seeds. An example of an organ-preferred and stress upregulated promoter is the At17.1 promoter, which drives gene expression in the roots and vascular system of soybean plants (Mazarei et al., Mol Plant Pathol 5:409-423, 2004). Other examples of tissue-preferred and organ-preferred promoters include, root-preferred, fruit-preferred, ovule-preferred, male tissue-preferred, seed-preferred, integument-preferred, tuber-preferred, stalk-preferred, pericarp-preferred, and leaf-preferred, stigma-preferred, pollen-preferred, anther-preferred, petal-preferred, sepal-preferred, pedicel-preferred, silique-preferred, and stem-preferred. Seed-preferred promoters are preferentially expressed during seed development and/or germination. For example, seed-preferred promoters can be embryo-preferred, endosperm-preferred, and seed coat-preferred (see Thompson et al., BioEssays 10:108, 1989). Examples of seed preferred promoters include cellulose synthase (celA), Cim1, gamma-zein, globulin-1, and maize 19 kD zein (cZ19B1).

Other suitable tissue-preferred or organ-preferred promoters include the napin-gene promoter from rapeseed (U.S. Pat. No. 5,608,152), the USP-promoter from Vicia faba (Baeumlein et al., Mol. Gen. Genet. 225:459-67, 1991), the oleosin-promoter from Arabidopsis (PCT Application No. WO 98/45461), the phaseolin-promoter from Phaseolus vulgaris (U.S. Pat. No. 5,504,200), the Bce4-promoter from Brassica (PCT Application No. WO 91/13980), or the legumin B4 promoter (LeB4; Baeumlein et al., Plant Journal, 2:233-9, 1992), as well as promoters conferring seed-specific expression in monocot plants including maize, barley, wheat, rye, and rice. Suitable promoters are the Ipt2 or Ipt1-gene promoter from barley (PCT Publication Nos. WO 95/15389 and WO 95/23230) or those described in PCT Publication No. WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene, and rye secalin gene). Other promoters useful in the invention include the major chlorophyll a/b binding protein promoter, histone promoters, the Ap3 promoter, the β-conglycin promoter, the napin promoter, the soybean lectin promoter, the maize 15 kD zein promoter, the 22 kD zein promoter, the 27 kD zein promoter, the γ-zein promoter, the waxy, shrunken 1, shrunken 2, and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), as well as synthetic or other natural promoters.

### Vectors

A polynucleotide encoding a silicon transporter (e.g., any of those described herein such as a polynucleotide operably linked to a promoter) may be part of an expression vector. Any suitable vector known in the art may be used. The vector may be an autonomously replicating vector, i.e., a vector existing as an extrachromosomal entity, the replication of which is independent of chromosomal replication, e.g., a plasmid. Alternatively, the vector may be one which, when introduced into a host cell, is integrated into the host cell genome and replicated simultaneously with the chromosomes into which it has been integrated.

Plant expression vectors can include (1) a cloned plant gene (e.g., a silicon transporter gene) under the transcriptional control of 5′ and optionally 3′ regulatory sequences (e.g., a promoter such as a promoter described herein). The vector may also include a dominant selectable marker. Such plant expression vectors may also contain, if desired, a promoter regulatory region (for example, one conferring inducible or constitutive, pathogen- or wound-induced, environmentally- or developmentally-regulated, or cell- or tissue-specific expression), a transcription initiation start site, a ribosome binding site, an RNA processing signal, a transcription termination site, and/or a polyadenylation signal.

Plant expression vectors may also optionally include RNA processing signals, e.g., introns, which have been shown to be important for efficient RNA synthesis and accumulation. The location of the RNA splice sequences can dramatically influence the level of transgene expression in plants. An intron may therefore be positioned upstream or downstream of a silicon transporter coding sequence in the transgene to alter levels of gene expression.

In addition to the aforementioned 5′ regulatory control sequences, the expression vectors may also include regulatory control regions which are generally present in the 3′ regions of plant genes. For example, the 3′ terminator region may be included in the expression vector to increase stability of the mRNA. One such terminator region may be derived from the PI-II terminator region of potato. In addition, other commonly used terminators are derived from the octopine or nopaline synthase signals.

The plant expression vector also typically contains a dominant selectable marker gene used to identify those cells that have become transformed. Useful selectable genes for plant systems include the aminoglycoside phosphotransferase gene of transposon Tn5 (Aph II), genes encoding antibiotic resistance genes, for example, those encoding resistance to hygromycin, kanamycin, bleomycin, neomycin, G418, streptomycin, or spectinomycin. Genes required for photosynthesis may also be used as selectable markers in photosynthetic-deficient strains. Finally, genes encoding herbicide resistance may be used as selectable markers; useful herbicide resistance genes include the bar gene encoding the enzyme phosphinothricin acetyltransferase and conferring resistance to the broad-spectrum herbicide Basta® (Bayer Cropscience Deutschland GmbH, Langenfeld, Germany). Other selectable markers include genes that provide resistance to other such herbicides such as glyphosate and the like, and imidazolinones, sulfonylureas, triazolopyrimidine herbicides, such as chlorosulfron, bromoxynil, dalapon, and the like. Furthermore, genes encoding dihydrofolate reductase may be used in combination with molecules such as methatrexate.

Efficient use of selectable markers is facilitated by a determination of the susceptibility of a plant cell to a particular selectable agent and a determination of the concentration of this agent which effectively kills most, if not all, of the transformed cells. Some useful concentrations of antibiotics for tobacco transformation include, for example, 20-100 μg/ml (kanamycin), 20-50 μg/ml (hygromycin), or 5-10 μg/ml (bleomycin). A useful strategy for selection of transformants for herbicide resistance is described, for example, by Vasil (Cell Culture and Somatic Cell Genetics of Plants, Vol I, II, III Laboratory Procedures and Their Applications Academic Press, New York, 1984).

In addition to a selectable marker, it may be desirable to use a reporter gene. In some instances, a reporter gene may be used without a selectable marker. Reporter genes are genes which are typically not present or expressed in the recipient organism or tissue. The reporter gene typically encodes for a protein which provide for some phenotypic change or enzymatic property. Examples of such genes are provided in Weising et al. (Ann. Rev. Genetics 22:421-478, 1988), which is incorporated herein by reference. Preferred reporter genes include without limitation glucuronidase (GUS) gene and GFP genes.

### Genetic Transformations of Plants

Any method for genetic transformation can be used to insert a polynucleotide encoding a silicon transporter into a plant. In some cases, it may be desirable to transform a plant with a silicon influx transporter, a silicon efflux transporter, or with both (e.g., any of the transporters described herein). Methods for the transformation of many plants, including soybeans, are well known to those of skill in the art. For example, techniques which may be employed for the genetic transformation of soybeans include electroporation, microprojectile bombardment, Agrobacterium-mediated transformation and direct DNA uptake by protoplasts.

To effect transformation by electroporation, one can employ either friable tissues, such as a suspension culture of cells or embryogenic callus or alternatively one may transform immature embryos or other organized tissue directly. In this technique, one can partially degrade the cell walls of the chosen cells by exposing them to pectin-degrading enzymes (pectolyases) or mechanically wound tissues in a controlled manner.

Protoplasts can also be employed for electroporation transformation of plants (Bates, Mol. Biotechnol., 2:135-145, 1994; Lazzeri, Methods Mol. Biol., 49:95-106, 1995). For example, the generation of transgenic soybean plants by electroporation of cotyledon-derived protoplasts is described in PCT Publication No. WO 92/17598, hereby incorporated by reference. A particularly efficient method for delivering transforming DNA segments to plant cells is microprojectile bombardment. Here, particles are coated with nucleic acids and delivered into cells by a propelling force. Exemplary particles include those comprised of tungsten, platinum, and preferably, gold. For the bombardment, cells in suspension are concentrated on filters or solid culture medium. Alternatively, immature embryos or other target cells can be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target soybean cells. The screen disperses the particles so that they are not delivered to the recipient cells in large aggregates. The smaller aggregates are believed to reduce the damage inflicted on cells by larger projectiles, thus resulting in higher transformation efficiency. Microprojectile bombardment techniques are widely applicable, and may be used to transform virtually any plant species (e.g., soybean or any plant described herein). The application of microprojectile bombardment for the transformation of soybeans is described, for example, in U.S. Pat. No. 5,322,783, hereby incorporated by reference.

Agrobacterium-mediated transfer is another widely used system for introducing gene loci into plant cells. An advantage of the technique is that DNA can be introduced into whole plant tissues, thereby bypassing the need for regeneration of an intact plant from a protoplast. Modern Agrobacterium transformation vectors are capable of replication in E. coli as well as Agrobacterium, allowing for convenient manipulations (Klee et al., Bio. Tech., 3:637-642, 1985). Recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti genes can be used for transformation. Agrobacterium-mediated transformation is described in U.S. Pat. Nos. 6,384,301 and 6,037,522, hereby incorporated by reference.

In those plant strains where Agrobacterium-mediated transformation is efficient, it is the method of choice because of the facile and defined nature of the gene locus transfer. The use of Agrobacterium-mediated plant integrating vectors to introduce DNA into plant cells is well known in the art (Fraley et al., Bio. Tech., 3:629-635, 1985; U.S. Pat. No. 5,563,055). Use of Agrobacterium in the context of soybean transformation has been described, for example, by Chee et al. (Methods Mol. Biol., 44:101-119, 1995) and in U.S. Pat. No. 5,569,834, each of which is hereby incorporated by reference.

Transformation of plant protoplasts also can be achieved using methods based on calcium phosphate precipitation, polyethylene glycol treatment, electroporation, and combinations of these treatments (see, e.g., Potrykus et al., Mol. Gen. Genet., 199:169-177, 1985; Omirulleh et al., Plant Mol. Biol., 21:415-428, 1993; Fromm et al., Nature, 319(6056):791-793., 1986; Uchimiya et al., Mol. Gen. Genet., 204:204-207, 1986; Marcotte et al., Nature, 335:454-457, 1988). The demonstrated ability to regenerate soybean plants from protoplasts makes each of these techniques applicable to soybean (Dhir et al., Plant Cell Rep., 10:97-101, 1991).

### Plants

Any plant may be used in the present invention. In certain embodiments, a plant that naturally does not accumulate high levels of silicon is used. Many plants do not efficiently accumulate silicon including soybean. In other embodiments, it may be desirable to increase silicon uptake in a plant that efficiently accumulates silicon (e.g., rice or a grassy plant such as wheat, oat, sorghum, or barley). Plants that may be used in the invention include a monocotyledenous crop plant such as barley, maize, oats, rice, rye, sorghum, and wheat; and a member of the grass family of Poaceae, such as Phleum spp., Dactylis spp., Lolium spp., Festulolium spp., Festuca spp., Poa spp., Bromus spp., Agrostis spp., Arrhenatherum spp., Phalaris spp., and Trisetum spp., for example, Phleum pratense, Phleum bertolonii, Dactylis glomerata, Lolium perenne, Lolium multiflorum, Lolium multiflorum westervoldicum, Festulolium braunii, Festulolium loliaceum, Festulolium holmbergii, Festulolium pabulare, Festuca pratensis, Festuca rubra, Festuca rubra rubra, Festuca rubra commutata, Festuca rubra trichophylla, Festuca duriuscula, Festuca ovina, Festuca arundinacea, Poa trivialis, Poa pratensis, Poa palustris, Bromus catharticus, Bromus sitchensis, Bromus inermis, Deschampsia caespitosa, Agrostis capilaris, Agrostis stolonifera, Arrhenatherum elatius, Phalaris arundinacea, and Trisetum flavescens; and a dicotyledenous plant, such as alfalfa, carrot, cotton, potato, sweet potato, oilseed rape, radish, soybean, sugarbeet, sugar cane, sunflower, tobacco, and turnip; vegetables such as asparagus, bean, carrot, chicory coffee, celery, cucumber, eggplant, fennel, leek, lettuce, garlic, onion, papaya, pea, pepper, spinach, squash, pumpkin, and tomato; vegetable brassicas such as brussel sprouts, broccoli, cabbage, and cauliflower; fruits, such as avocado, banana, blackberry, blueberry, grapes, mango, melon, nectarine, orange, papaya, pineapple, raspberry, strawberry; rosaceous fruits such as apple, apricot, peach, pear, cherry, plum, and quince; herbs such as anise, basil, bay laurel, caper, caraway, cayenne pepper, celery, chervil, chives, coriander, dill, horseradish, lemon balm, liquorice, marjoram, mint, oregano, parsley, rosemary, sesame, tarragon, and thyme; woody species, such as eucalyptus, oak, pine, and poplar.

### Screening of Transformed Plants

Once a plant is transformed with a Si-transport gene, screening can be accomplished by any means known in the art. In some cases, screening is performed using the silicon detection techniques described below. Other screening techniques may involve screening for uptake, transport, or efflux of ⁶⁸Ge. As described above, ⁶⁸Ge has been used to evaluate silicon uptake in Xenopus oocytes. Such an approach can also be used to evaluate silicon uptake in higher plants, as molar ratios between ⁶⁸Ge and silicon have been observed to remain constant following uptake in different plant tissues (Nikolic et al., Plant Physiol 143:495-503, 2007).

In other embodiments, the plants can be screened for resistance to one or more biotic stresses, one or more abiotic stresses, or any combination thereof. In one example, soybean plants transformed with a silicon influx transport, a silicon efflux transporter, or both are screened for resistance to soybean rust (Phakopsora pachyrhyzi). In general, untransformed plants and transformed plants are grown in the presence of a stress (e.g., any described herein), and the effect of silicon transporter expression on stress resistance is determined by measuring a phenotypic response to the stress (e.g., growth, survival, weight, yield), where an improvement in the phenotypic response (e.g., increased growth, higher rate of survival) in the transformed plant as compared to the non-transformed plants indicates that the transformation with the silicon transporter is beneficial.

Any appropriate abiotic stress may be used to evaluate the effect of transforming a cell or plant with a silicon transporter. Exemplary abiotic stresses include salinity, temperature (e.g., heat or cold), oxidative stress, insufficient or excess water (waterlogging or drought), insufficient or excessive minerals (e.g., mineral toxicity), physical stress (e.g., wind). Health or growth parameters, such as height, weight, yield, or survival are recorded and compared to untransformed control plants subjected to the same stress.

Alternatively, plants may be subjected to biotic stresses, such as bacteria, fungus, or an insect. Any biotic stress known in the art may be used to screen plants. Other pathogens affecting soybean include Phytophthora megasperma fsp. glycinea, Macrophomina phaseolina, Rhizoctonia solani, Sclerotinia sclerotiorum, Fusarium oxysporum, Diaporthephaseolorum var. sojae (Phomopsis sojae), Diaporthephaseolorum var. caulivora, Sclerotium rolfsii, Cercospora kikuchii, Cercospora sojina, Peronospora manshurica, Colletotrichum dematium (Colletotichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Pythium aphanidermatum, Pythium ultimum, Pythium debaryanum, Tomato spotted wilt virus, Heterodera glycines, and Fusarium solani.

In general, exemplary fungi include Alternaria (Alternaria brassicola; Alternaria solani), Ascochyta (Ascochyta pisi); Botrytis (Botrytis cinerea); Cercospora (Cercospora kikuchii; Cercospora zeae-maydis); Colletotrichum (Colletotrichum lindemuthianum); Diplodia (Diplodia maydis); Erysiphe (Erysiphe graminis f. sp. graminis; Erysiphe graminis f. sp. hordei); Fusarium (Fusarium nivale; Fusarium oxysporum; Fusarium graminearum; Fusarium culmorum; Fusarium solani; Fusarium moniliforme; Fusarium roseum); Gaeumanomyces (Gaeumanomyces graminis f, sp. tritici); Helminthosporium (Helminthosporium turcicum; Helminthosporium carbonum; Helminthosporium maydis); Macrophomina (Macrophomina phaseolina); Magnaporthe (Magnaporthe grisea); Nectria (Nectria haematococca); Peronospora (Peronospora manshurica; Peronospora tabacina); Phoma (Phoma betae); Phymatotrichum (Phymatotrichum omnivorum); Phytophthora (Phytophthora cinnamomi; Phytophthora cactorum; Phytophthora phaseoli; Phytophthora parasitica; Phytophthora citrophthora; Phytophthora megasperma f. sp. sojae; Phytophthora infestans); Plasmopara (Plasmopara viticola); Podosphaera (Podosphaera leucotricha); Puccinia (Puccinia sorghi; Puccinia striiformis; Puccinia graminis f. sp. tritici; Puccinia asparagi; Puccinia recondita; Puccinia arachidis); Pyrenophora (Pyrenophora tritici-repentis); Pyricularia (Pyricularia oryzae); Pythium (Pythium aphanidermatum; Pythium ultimum); Rhizoctonia (Rhizoctonia solani; Rhizoctonia cerealis); Sclerotium (Sclerotium rolfsii); Sclerotinia (Sclerotinia sclerotiorum); Septoria (Septoria lycopersici; Septoria glycines; Septoria nodorum; septoria tritici); Thielaviopsis (Thielaviopsis basicola); Uncinula (Uncinula necator); Venturia (Venturia inaequalis); and Verticillium (Verticillium dahliae; Verticillium albo-atrum).

Examples of rusts include rust caused by Basidiomycetes of the order Uredinales; Puccinia (P. graminis, P. stiiformis, P. recondita, P. hordei, P. coronata, P. sorghi., P. polysora, P. purpurea, P. sacchari P. kuehnii, P. stakmanii, P. asparagi, P. chrysanthemi, P. malvacearum, and P. antirrhini); Gymnosporangium (G. juniperi-virginianae, G. globosum); Hemileia (H. vastatrix); Phragmidium; Uromyces (U. caryophyllinus); Cronartium (C. ribicola, C. quercuum f. sp. fusiforme, C. quercuum f. sp. virginianae, C. comptoniae, C. comandrae, C. strobilinum); Melampsora (M. lini); Coleosporium (C. asterinum); Gymnoconia; Phakopsora (P. pahyrhizi) and Tranzschelia.

In one example, transformed plants and untransformed controls are grown hydroponically in a nutritive solution containing 1.7 mM Si, the maximum solubility of Si in solution. Plant roots and aerial parts are harvested, and their Si content is measured by techniques described below.

### Si Detection, Localization, and Quantification

Once a cell or a plant (e.g., a Xenopus oocyte or a plant described herein) has been transformed to express a silicon transporter, it may be desirable to measure the amount of silicon in the cell or plant. Several methods exist to determine a Si content in different substrates. Typically, when measuring silicon in biological sample, Si quantification is performed through a spectrometric analysis, which, in some cases, may result in destruction of the sample.

One non-destructive analytical method is X-ray fluorescence spectroscopy. This technique allows detection and quantification of Si in biological material, for example, by measuring and analyzing the secondary radiation emitted from a substrate excited with a X-ray source. Prior to visualization, samples are frozen to −80° C. and then lyophilized. Once completely dry, they are attached to carbon SEM stubs and coated with gold. Samples are then submitted to X-rays and the secondary radiation is recorded and quantified.

Other spectrometric analyses are performed on treated samples, for which Si is solubilized, usually in an acid solution. Samples can be prepared by autoclave-induced digestion, acid digestion, microwave assisted acid digestion, or NaOH fusion. The resulting solution can then be analyzed using a colorimetric method (either yellow silicomolybdic acid or blue silicomolybdous acid procedure), atomic absorption spectrometry, or inductively coupled plasma (ICP).

ICP is reported to have the lowest detection limit (3 ppb) and the greatest precision. Thus, it may be the method of choice when dealing with Si quantification, where solubilization is possible. Once a sample is prepared for an ICP analysis, it is converted into aerosol with a nebulizer. A desolvation/volatilization phase occurs, in which water is driven off while solid and liquid fractions are converted into gases. Then an atomization phase takes place where gas phase bonds are broken. This step produces a plasma which requires a high temperature (5000 to 8000° C.) to maintain and an inert chemical environment, usually provided by Argon. The plasma is then excited by X-rays and releases electromagnetic radiation (hv) in an element-specific wavelength. For instance, Si emits at 251,611 nm. A detector then measures the light emitted and quantifies it. ICP can thus be used to assess Si-transport efficiency following oocyte transformation and also to measure Si absorption in plants (e.g., transformed or untransformed).

Indirect, analytical methods for measuring silicon uptake using a germanium tracer may be used. This approach is described using oocytes above but can also be applied to higher plants. Using small amounts of radioactive germanium (⁶⁸Ge) as a tracer can be used as a means for measuring silicon uptake (see, e.g., Nikolic et al., Plant Physiol 143:495-503, 2007).

Finally, silicon influx or efflux can be measured by another method following oocyte transformation and incubation in a solution containing silicon: oocytes were washed, solubilized in HNO₃ and the silicon content was directly quantified by atomic-absorption (AA) spectrometry. Atomic-absorption spectroscopy uses the absorption of light to measure the concentration of gas-phase atoms. Because samples are usually liquids or solids, the analyte atoms or ions must be vaporized in a flame or graphite furnace. The atoms absorb ultraviolet or visible light and make transitions to higher electronic energy levels. The analyte concentration is determined from the amount of absorption. Silicon concentration measurements were determined from a working curve after calibrating the instrument with standards of known concentration.

### Synthesis of Silicon Transporter Polypeptides

Nucleic acids that encode silicon transporter polypeptides or fragments thereof may be introduced into various cell types or cell-free systems for expression, thereby allowing purification of these polypeptides for biochemical characterization, large-scale production, antibody production, and patient therapy.

Eukaryotic and prokaryotic silicon transporter expression systems may be generated in which a silicon transporter gene sequence is introduced into a plasmid or other vector, which is then used to transform living cells. Constructs in which the silicon transporter cDNA contains the entire open reading frame inserted in the correct orientation into an expression plasmid may be used for protein expression. Alternatively, portions of the silicon transporter gene sequences, including wild-type or mutant silicon transporter sequences, may be inserted. Prokaryotic (e.g., E. coli) and eukaryotic expression systems allow various important functional domains of the silicon transporter proteins to be recovered, if desired, as fusion proteins, and then used for binding, structural, and functional studies and also for the generation of appropriate antibodies.

Typical expression vectors contain promoters that direct the synthesis of large amounts of mRNA corresponding to the inserted silicon transporter nucleic acid in the plasmid-bearing cells. They may also include a eukaryotic or prokaryotic origin of replication sequence allowing for their autonomous replication within the host organism, sequences that encode genetic traits that allow vector-containing cells to be selected for in the presence of otherwise toxic drugs, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

Expression of foreign sequences in bacteria, such as Escherichia coli, requires the insertion of the silicon transporter nucleic acid sequence into a bacterial expression vector. Such plasmid vectors contain several elements required for the propagation of the plasmid in bacteria, and for expression of the DNA inserted into the plasmid. Propagation of only plasmid-bearing bacteria is achieved by introducing, into the plasmid, selectable marker-encoding sequences that allow plasmid-bearing bacteria to grow in the presence of otherwise toxic drugs. The plasmid also contains a transcriptional promoter capable of producing large amounts of mRNA from the cloned gene. Such promoters may be (but are not necessarily) inducible promoters that initiate transcription upon induction. The plasmid also preferably contains a polylinker to simplify insertion of the gene in the correct orientation within the vector.

Once the appropriate expression vectors containing a silicon transporter gene, fragment, fusion, or mutant are constructed, they are introduced into an appropriate host cell by transformation techniques, such as, but not limited to, calcium phosphate transfection, DEAE-dextran transfection, electroporation, microinjection, protoplast fusion, or liposome-mediated transfection. The host cells that are transfected with the vectors of this invention may include (but are not limited to) E. coli or other bacteria, yeast, fungi, plant cells, insect cells (using, for example, baculoviral vectors for expression in SF9 insect cells), or cells derived from mice, humans, or other animals. In vitro expression of silicon transporter proteins, fusions, polypeptide fragments, or mutants encoded by cloned DNA may also be used. Those skilled in the art of molecular biology will understand that a wide variety of expression systems and purification systems may be used to produce recombinant silicon transporter proteins and fragments thereof.

Once a recombinant protein is expressed, it can be isolated from cell lysates using protein purification techniques such as affinity chromatography. Once isolated, the recombinant protein can, if desired, be purified further, e.g., by high performance liquid chromatography (HPLC; e.g., see Fisher, Laboratory Techniques In Biochemistry And Molecular Biology, Work and Burdon, Eds., Elsevier, 1980).

Polypeptides of the invention, particularly short silicon transporter fragments can also be produced by chemical synthesis (e.g., by the methods described in Solid Phase Peptide Synthesis, 2nd ed., 1984, The Pierce Chemical Co., Rockford, Ill.).

The following examples are intended to illustrate, rather than limit, the invention.

### EXAMPLE 1

### Preparation of Total cDNA Extract from Wheat Roots

Wheat plants cv. HY644 were grown in hydroponic systems, set up to immerse roots in a nutrient solution for 15 minutes every 30 minutes. Each system contained 12 pots, each containing 2-3 seeds sown in vermiculite. The systems were kept in a greenhouse (16 h light at 22° C. and 8 h dark at 18° C., 80% humidity). Seeds were germinated in distilled water, which was replaced by Hoagland nutritive solution at the 2-3 leaf stage. This solution was replaced every other week. Once mature, wheat roots were carefully recovered and immediately frozen in liquid nitrogen. Frozen roots were crushed in a clean, autoclaved mortar. Total mRNA was then extracted from the root powder using an RNA extraction kit (QIAgen); the RNA was stored at −80° C. until use. Five μl of a 300 ng/μl of total mRNAs were added to a mix containing 2 μl of oligodT18 (5 μM), 1 μl of dNTP (10 mM) and 4.5 μl of RNAse free water then incubated 5 min at 65° C. followed by 2 min on ice. Four μl of TP 5×, 1 μl of DTT, 1 μl of RNase OUT RNase inhibitor (Invitrogen) and 1 μl of Superscript III reverse transcriptase (Invitrogen) were added to the RNA solution and incubated 50 min at 55° C. to allow oligodT primer extension. The mixtures was then incubated for 15 min at 70° C. to inactive the reverse transcriptase. 2 μl of a 1 U/μl Ribonuclease H (Roche) were added to the cDNA preparation and incubated 20 min at 37° C. to hydrolyze RNA. The cDNA sample was then purified with a PCR purification kit (QIAgen) to remove all traces of dNTP, oligodT and RNA fragments.

### EXAMPLE 2

### Amplification and Cloning of a Silicon Transporter Fragment in Wheat

100 ng of wheat cDNA obtained as described in Example 1 were added to a mix containing 1 μl of dNTP (10 mM), 2.5 μl pf TP 10×, 1.5 μl of 25 mM MgCl₂, 12.75 μl of ddH₂O, 0.25 μl of HotStart Taq DNA polymerase (Eppendorf), 1 μl of 5 μM primer 1F (SEQ ID NO:7), and 1 μl of 5 μM primer 2R (SEQ ID NO:8). PCR was conducted using the following conditions. Initial denaturation was performed at 94° C. for 2 min, followed by 40 cycles of denaturation (94° C., 30 s), annealing (62° C., 30 s) and primer extension (72° C., 1 min), and one final extension (72° C., 10 min).

Agarose gel electrophoresis of the PCR product demonstrated a unique band of the expected size. Three μl of the purified PCR product were added to 5 μl of TP 2× Rapid Ligation Buffer, 1 μl of 50 ng/μl pGEM-T T plasmid (Promega), and 1 μl of T4 DNA ligase (Promega) and incubated at 4° C. overnight. Five μl of the ligation reaction were placed on a nylon membrane and desalted 30 min with 30% glycerol. The desalted ligation was added to 50 μl of DH5α E. coli competent cells in a sterile electroporation cuvette placed in an electroporator (Biorad). The electroporation conditions were (200Ω, 2.5 KV). 950 μl of pre-cooled LB medium were added to electoporated cells and the solution was incubated 30 min at 37° C. before plating different volumes of the transformation on LB+100 μg/ml ampicillin Petri plates. Ten μl of IPTG and 40 μl of X-Gal were added in each Petri plate to allow the selection of positive transformants using the white/blue test. Plates were incubated overnight at 37° C. White colonies were recovered and subjected to PCR analysis to confirm the presence of the correct insert.

Twenty-four putative transformants were recovered and placed in a PCR mix containing 0.8 μl dNTP (10 mM), 2 μl TP 10×, 1.2 μl MgCl₂, 15.4 μl ddH₂O, 0.2 μl Taq DNA polymerase, 0.2 μl M13F primer (50 μM) and 0.2 μl M13R primer (50 μM). PCR was conducted using the following conditions. Initial denaturation was performed at 94° C. for 2 min, followed by 24 cycles of denaturation (94° C., 30 s), annealing (55° C., 30 s) and primer extension (68° C., 2 min), and one final extension (62° C., 10 min).

Agarose gel electrophoresis of the PCR product demonstrated the presence of several clones with an insert of the expected size. These good transformants were grown in 5 ml of liquid LB+100 μg/ml ampicillin overnight at 37° C. The culture was centrifuged for 10 min at 4° C., and the cell pellet was subjected to a plasmid purification kit (QIAgen). Recovered plasmids were sequenced at the “Service de Séquençage de l'Université Laval.”

### EXAMPLE 3

### 3′ RACE (Rapid Amplification of cDNA Ends) in Wheat

100 ng of wheat cDNA obtained as described in example 1 were added to a mix containing 1 μl of dNTP (10 mM), 2.5 μl pf TP 10×, 1.5 μl of 25 mMMgCl₂, 12.75 μl of ddH₂O, 0.25 μl of HotStart Taq DNA polymerase (Eppendorf), 1 μl of 5 μM ADApT primer (SEQ ID NO:16) and 1 μl of 5 μM BleR primer (SEQ ID NO:18). PCR was conducted using the following conditions. Initial denaturation was performed at 94° C. for 2 min, followed by 40 cycles of denaturation (94° C., 1 min), annealing (52° C., 30 s) and primer extension (72° C., 1 min), and one final extension (72° C., 10 min). The PCR product was diluted 100 times and submitted to another amplification with the same conditions but using 1 μl of 5 μM ADA primer (SEQ ID NO:17) and 1 μl of 5 μM BleRNested (5 μM, 5′-CCTGCGAAGATGGAGGTAA-3′).

The PCR product analyzed on agarose gel electrophoresis demonstrated a unique band of the expected size. The PCR product was cloned for sequencing as described in Example 2.

### EXAMPLE 4

### Expression of cRNAs in Xenopus oocytes and Quantification of Si Content

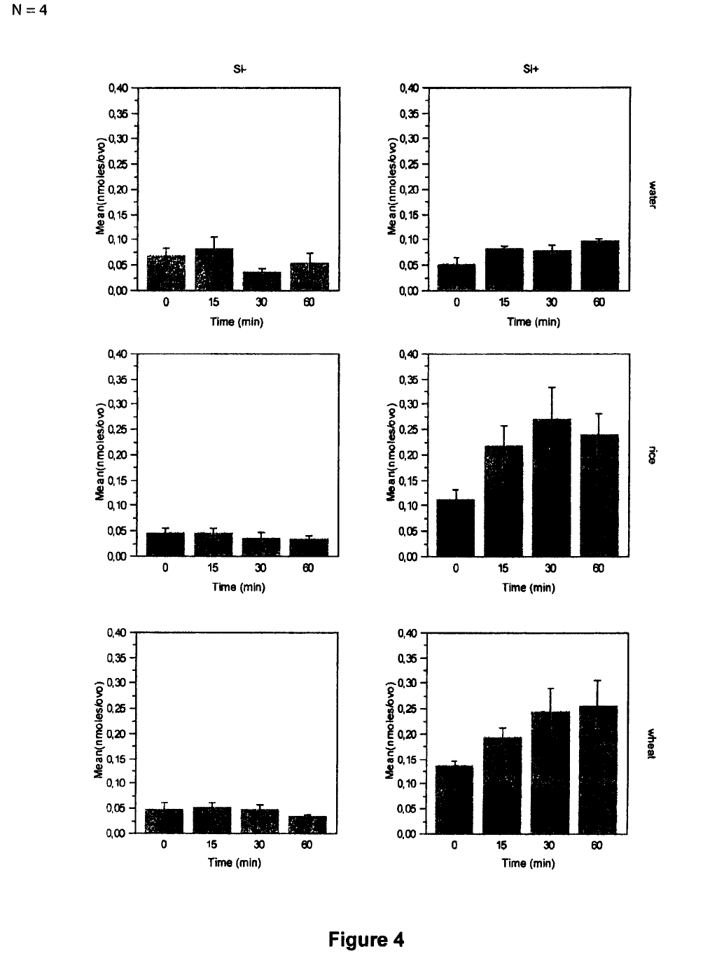

Oocytes were taken from Xenopus laevis females. After dissection, a set of oocytes was transferred to a physiological medium with antibiotics. They were kept at 18° C. until use for up to 72 h. cRNA was dissolved in RNase-free water. Twenty to 50 nl of the injection fluid (200 ng/μl cRNA) was injected into each prepared oocyte using a micromanipulator. The oocytes were then incubated at 18° C. for about 48 h to allow protein synthesis and membrane integration. Si uptake measurement was then performed. Si was added to the physiological medium to reach a 1.7 mM concentration. After 0, 15, 30, or 60 min, oocytes were rinsed to remove external Si. Si content in oocytes was then measured by atomic absorption spectrometry, as described above.

Whereas in water injected oocytes, the concentration remained constant over the experimental period, both in wheat or rice SIIT1 transformed oocytes, a higher concentration of silicon was detected over time (FIG. 4). These results indicate that the sequence of SIIT1 identified in wheat is a homolog of the rice SIIT1 and can function as a silicon transporter.

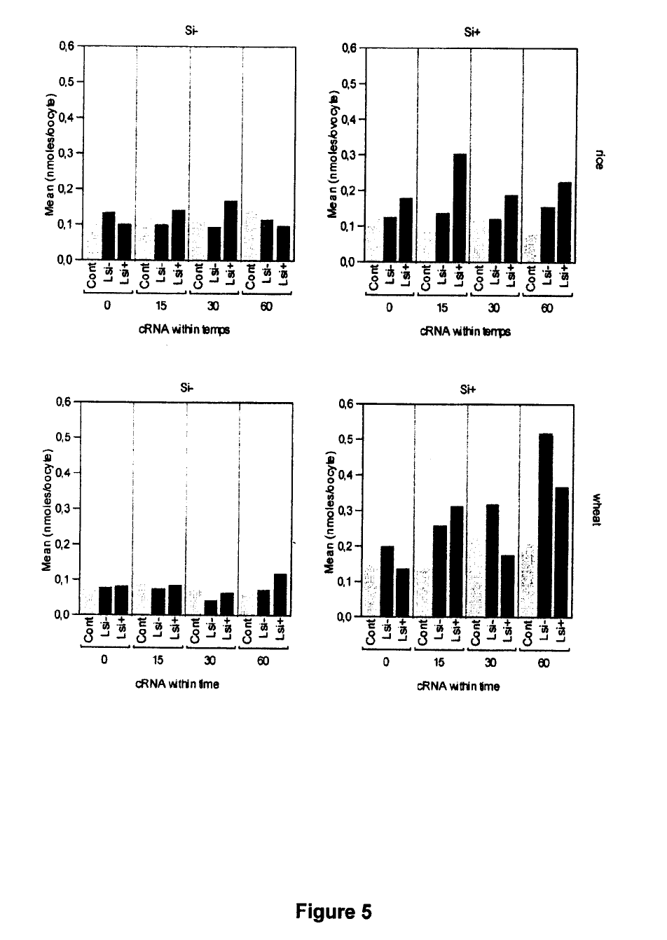

A comparison between wild type and mutant SIIT1 was also performed in both rice and wheat. The rice SIIT1 mutant showed characteristics of a defective silicon transporter with a lower transport activity than the wild protein (FIG. 5).

Suprisingly, after 30 min of incubation, oocytes injected with the wheat mutant SIIT1 exhibited a greater activity than the wild protein. This would indicate that mutations (changes) in the protein sequence can lead to either lower or higher Si transport activity.

### EXAMPLE 5

### Transformation of Soybean Plants with Si-Transport Gene Genes

Several genes can be introduced into a plant during a single transformation event. For the present invention, one example of a DNA construct consisting of an Agrobacterium p-CAMBIA plasmid containing the following sequence can be introduced in the plant genome using kanamycin resistance as a selection marker: CaMV 35 S promoter—kanamycin resistance gene—terminator—CaMV 35 S promoter—SIIT1 gene—terminator—CaMV 35 S promoter—SIIT2 gene—terminator—CaMV 35 S promoter—SIET1 gene—terminator. (see, e.g., Dans and Wei Plant Science 173:381-389, 2007 for an example of soybean transformation with two insect resistance genes). The DNA construction is introduced in Agrobacterium tumefasciens bacteria.

Soybean calluses are co-cultured with the Agrobacterium. The plant cells are then transferred to a culture medium containing the selection marker, kanamycin in this example. Only the plant cells that have integrated the DNA construction and expressed the kanamycin-resistance gene will grow.

Additional controls can be performed using PCR. To verify that the SIIT1, SIIT2, and SIET1 genes are integrated in the plant genome, total plant DNA is extracted, and PCR is performed using primers specific for either the SIIT1, SIIT2, or SIET1 genes. To verify that SIIT1, SIIT2, and SIET1 genes are expressed in the plant, root RNA is extracted and reverse-transcribed in complementary DNA (cDNA, as described in Example 1). PCR is then performed on the cDNA using primers specific for the SIIT1, SIIT2, and SIET1 genes using standard methods.

These plants, once grown up, can be tested for increased resistance to various biotic and abiotic stresses, including soybean rust. Desirably, such transformed plants will have increased resistance to such stresses, including increased resistance to soybean rust.

### Other Embodiments

All patents, patent applications including U.S. Provisional Application No. 61/070,528, filed Mar. 24, 2008, and publications mentioned in this specification are herein incorporated by reference to the same extent as if each independent patent, patent application, or publication was specifically and individually indicated to be incorporated by reference.

What is claimed is:

1. A substantially pure polynucleotide comprising a nucleic acid sequence with at least 85% identity to a nucleotide sequence selected from the group consisting of SEQ ID NOS:15, 4, 9, 12, 13, 14, 33, 50, 52, 67, nucleotides 124-919 of SEQ ID NO:21, nucleotides 146-694 of SEQ ID NO:22, and nucleotides 124-1014 of SEQ ID NO:32.

2. A substantially pure polynucleotide including a nucleic acid sequence with at least 95% identity to the nucleotide sequence of SEQ ID NO:56 or at least 90% identity to the nucleotide sequence of SEQ ID NO:58 or SEQ ID NO:59.

3. The polynucleotide of claim 1 or 2, wherein expression of the polypeptide encoded by said polynucleotide in a cell is capable of increasing silicon transport into said cell.

4. The polynucleotide of claim 1 or 2, wherein said identity is at least 95%.

5. The polynucleotide of claim 4, wherein said identity is at least 99%.

6. The polynucleotide of claim 5 comprising a nucleotide sequence selected from the group consisting of SEQ ID NOS:4, 9, 12, 13, 14, 15, 33, 50, 52, 67, nucleotides 124-919 of SEQ ID NO:21, nucleotides 146-694 of SEQ ID NO:22, and nucleotides 124-1014 of SEQ ID NO:32.

7. The polynucleotide of claim 5 comprising a nucleotide sequence selected from the group consisting of SEQ ID NOS:56, 58, and 59.

8. The polynucleotide of claim 1 or 2, wherein said polynucleotide is less than 20 kB in length.

9. The polynucleotide of claim 1 or 2 operably linked to a promoter.

10. The polynucleotide of claim 9, wherein said promoter is capable of expression in a plant cell.

11. The polynucleotide of claim 10, wherein said plant cell is a root cell.

12. A vector comprising the polynucleotide of claim 9.

13. The vector of claim 12, further comprising a second polynucleotide encoding a silicon efflux transporter.

14. The vector of claim 13, wherein said second polynucleotide has at least 80% identity to a nucleic acid sequence selected from the group consisting of SEQ ID NOS:28, 29, 71, and 73.

15. A cell comprising the vector of claim 12.

16. The cell of claim 15, wherein said cell is a plant cell.

17. The cell of claim 16, wherein said plant cell is a soybean plant cell.

18. A seed comprising the cell of claim 15.

19. A substantially pure polypeptide encoded by the polynucleotide of claim 1 or 2.

20. A substantially pure polynucleotide comprising a nucleic acid sequence with at least 80% identity to a nucleotide sequence selected from the group consisting of SEQ ID NOS:29, 71, and 73.

21. The polynucleotide of claim 20, wherein expression of the polypeptide encoded by said polynucleotide in a cell is capable of increasing silicon transport from said cell.

22. The polynucleotide of claim 20, wherein said identity is at least 95%.

23. The polynucleotide of claim 22, wherein said identity is at least 99%.

24. The polynucleotide of claim 23 comprising a nucleotide sequence selected from the group consisting of SEQ ID NOS:29, 71, and 73.

25. The polynucleotide of claim 20, wherein said polynucleotide is less than 20 kB in length.

26. The polynucleotide of claim 20 operably linked to a promoter.

27. The polynucleotide of claim 26, wherein said promoter is capable of expression in a plant cell.

28. The polynucleotide of claim 27, wherein said plant cell is a root cell.

29. A vector comprising the polynucleotide of claim 26.

30. The vector of claim 29, further comprising a second polynucleotide having at least 80% identity to a nucleic acid sequence selected from the group consisting of SEQ ID NOS:3, 4, 9, 12, 13, 14, 15, 33, 50, 52, 53, 55, 56, 57, 58, 59, 67, nucleotides 124-919 of SEQ ID NO:21, nucleotides 146-694 of SEQ ID NO:22, and nucleotides 124-1014 of SEQ ID NO:32.

31. A cell comprising the vector of claim 29.

32. The cell of claim 31, wherein said cell is a plant cell.

33. The cell of claim 32, wherein said plant cell is a soybean cell.

34. A seed comprising the cell of claim 31.

35. A substantially pure polypeptide encoded by the polynucleotide of claim 20.

36. A plant comprising a heterologous polynucleotide comprising a nucleic acid sequence having at least 85% identity to a nucleic acid sequence selected from the group consisting of SEQ ID NOS:4, 9, 12, 13, 14, 15, 33, 50, 52, 67, nucleotides 124-919 of SEQ ID NO:21, nucleotides 146-694 of SEQ ID NO:22, and nucleotides 124-1014 of SEQ ID NO:32.

37. The plant of claim 36, wherein said identity is at least 95%.

38. The plant of claim 37, wherein said identity is at least 99%.

39. The plant of claim 38, wherein said polynucleotide comprises a nucleic acid sequence selected from the group consisting of SEQ ID NOS:4, 9, 12, 13, 14, 15, 33, 50, 52, 67, nucleotides 124-919 of SEQ ID NO:21, nucleotides 146-694 of SEQ ID NO:22, and nucleotides 124-1014 of SEQ ID NO:32.

40. The plant of claim 36, wherein the polypeptide encoded by said heterologous polynucleotide increases the transport of silicon into at least one tissue within said plant upon expression.

41. The plant of claim 36, further comprising a second heterologous sequence having at least 80% identity to a polynucleotide encoding a silicon efflux transporter or a second silicon influx transporter.

42. The plant of claim 41, where said second sequence has at least 80% identity to at least one sequence selected from the group consisting of SEQ ID NO:28, 29, 71, 73, 55, 56, 57, 58, and 59.

43. The plant of claim 36, wherein said plant is a soybean plant.

44. A plant comprising a heterologous polynucleotide sequence having at least 80% identity to a nucleic acid sequence selected from the group consisting of SEQ ID NOS:29, 71, and 73.

45. The plant of claim 44, wherein said identity is at least 95%.

46. The plant of claim 45, wherein said identity is at least 99%.

47. The plant of claim 46, wherein said polynucleotide comprises a nucleic acid sequence selected from the group consisting of SEQ ID NO:29, 71, and 73.

48. The plant of claim 44, wherein the polypeptide encoded by said heterologous polynucleotide increases the transport of silicon from at least one tissue within said plant upon expression.

49. The plant of claim 44 further comprising a second heterologous sequence having at least 80% identity to a nucleic acid sequence encoding a silicon influx transporter.

50. The plant of claim 49, wherein said second heterologous sequence has at least 80% identity to at least one sequence selected from the group consisting of SEQ ID NOS:3, 4, 9, 12, 13, 14, 15, 33, 50, 52, 53, 55, 56, 57, 58, 59, 67, nucleotides 124-919 of SEQ ID NO:21, nucleotides 146-694 of SEQ ID NO:22, and nucleotides 124-1014 of SEQ ID NO:32.

51. The plant of claim 43, wherein said plant is a soybean plant.

52. A plant comprising a heterologous polynucleotide having at least 90% identity to the nucleic acid sequence of SEQ ID NO:56, 58, or 59.

53. The plant of claim 52, wherein said polynucleotide comprises the sequence of SEQ ID NO:56, 58, or 59.

54. The plant of claim 52, wherein said plant comprises a second heterologous polynucleotide encoding a silicon efflux transporter.

55. The plant of claim 54, wherein said second heterologous polynucleotide has at least 80% sequence identity to a sequence selected from the group consisting of SEQ ID NO:28, 29, 71, and 73.

56. The plant of claim 52, wherein said plant comprises a second heterologous polynucleotide encoding a silicon influx transporter.

57. The plant of claim 56, wherein said second heterologous polynucleotide has at least 80% sequence identity to a sequence selected from the group consisting of SEQ ID NOS:3, 4, 9, 12, 13, 14, 15, 33, 50, 52, 53, 67, nucleotides 124-919 of SEQ ID NO:21, nucleotides 146-694 of SEQ ID NO:22, and nucleotides 124-1014 of SEQ ID NO:32.

58. The plant of claim 56, wherein said plant comprises a third heterologous polynucleotide encoding a silicon efflux transporter.

59. The plant of claim 58, wherein said third heterologous polynucleotide is at least 80% identical to a sequence selected from the group consisting of SEQ ID NO:28, 29, 71, and 73.

60. A method of generating a plant with increased silicon uptake, said method comprising:
(a) providing a first vector comprising the polynucleotide comprising a nucleic acid sequence having at least 85% identity to a sequence selected from the group consisting of SEQ ID NO:4, 9, 12, 13, 14, 15, 33, 50, 52, 56, 58, 59, 67, nucleotides 124-919 of SEQ ID NO:21, nucleotides 146-694 of SEQ ID NO:22, and nucleotides 124-1014 of SEQ ID NO:32;
(b) transforming a plant cell with said vector; and
(c) growing a plant from said cell, wherein said plant expresses said polynucleotide, thereby generating a plant with increased silicon uptake.

61. The method of claim 60, wherein said vector further comprises a second sequence encoding a silicon efflux transporter.

62. The method of claim 60, wherein said cell is further transformed with a second vector comprising a second sequence encoding a silicon efflux transporter either simultaneously or sequentially with said first vector.

63. The method of claim 61 or 62, wherein said second sequence has at least 80% identity to a sequence selected from the group consisting of SEQ ID NO:28, 29, 71, and 73.

64. The method of claim 60, wherein said plant cell is a soybean cell.

65. A method of generating a plant with increased silicon transport, said method comprising:
(a) providing a first vector comprising the polynucleotide comprising a nucleic acid sequence having at least 80% identity to a nucleotide sequence of SEQ ID NO:29, 71, and 73;
(b) transforming a plant cell with said vector; and
(c) growing a plant from said cell, wherein said plant expresses said polynucleotide, thereby generating a plant with increased silicon transport.

66. The method of claim 65, wherein said vector further comprises a second sequence encoding a silicon influx transporter.

67. The method of claim 65, wherein said plant cell is further transformed with a second vector comprising a second sequence having at least 80% identity to a silicon influx transporter either simultaneously or sequentially with said first vector.

68. The method of claim 66 or 67, wherein said second sequence is selected from the group consisting of SEQ ID NO:3, 4, 9, 12, 13, 14, 15, 33, 50, 52, 53, 55, 56, 57, 58, 59, 67, nucleotides 124-919 of SEQ ID NO:21, nucleotides 146-694 of SEQ ID NO:22, and nucleotides 124-1014 of SEQ ID NO:32.

69. The method of claim 65, wherein said plant cell is a soybean cell.


SEQUENCE LISTING (74 sequences)

SEQ ID NO: 1 - DNA, 1409 bp, Oryza sativa
ggatcgacggagcgagcgagctagccagccagtgttagagcttgagctgcttgttcttcttctacctcctgcactcgcgtgctgcacaagtagctcagctagatagagcgtcagaaatggccagcaacaactcgagaacaaactccagggcgaactactccaacgagatccacgatctctccacggtgcagaacggcaccatgcctaccatgtactacggcgagaaggccatcgccgacttcttccctcctcacctcctcaagaaggtcgtgtcggaggtggtggccacgttcctgctggtgttcatgacgtgtggggcggcagggatcagcggcagcgacctgtctcgcatatcgcagctgggacagtcgatcgccggtggcctcatcgtgacggtgatgatctacgccgtcggccacatctccggcgcccacatgaaccccgccgtgacgctcgcgttcgccgtgttcaggcatttcccctggattcaggttccgttctactgggcggcgcagttcaccggagcgatatgcgcgtcgttcgtgctcaaggcggtgatccacccggtggatgtgatcggaaccaccacgcccgtggggccgcactggcactcgctcgtcgtcgaggtcatcgtgacgttcaacatgatgttcgtcacgctcgccgtcgccacggacacgagagcggtgggtgagttggccgggttggcggttggttccgcggtttgcattacgtccatcttcgcaggggcaatttcaggtggatcgatgaacccggcaaggacgctggggccggcgctggcgagcaacaagttcgacggcctgtggatctacttcctgggcccagtcatgggcacgctctcgggagcatggacctacaccttcatccgcttcgaggacacccccaaggaaggctcctcccagaagctctcctccttcaagctgcgccgcttgcggagccagcagtccatcgccgccgacgacgtcgacgagatggagaacatccaagtgtgataggacgacgagatgtcgtcgatcgtgtcctcttactcggaaaattaccagtcgatctcggtctcgtcatcactagctacgtctttgtgtgtgttttgtgtcgtgtcactgctgcttcgtacacgccggagagagttacataaaacgcgcgcgcgtgcacgggggagtggcgctagctttggttgcttggtggttcgtgtggggtacgtagattgctcgctctgttaattccggggctggagcctgctaattttgggcgtgtgcgtgtgccgcttgtgcatcagtctctgccgtcaaattgtgcgtgtgtgccaggtgtactatctctccagcagttgcttgttccacttcaattcacccccaaagtaattaaaaggatgtcacgttctctcccct

SEQ ID NO: 2 - DNA, 1340 bp, Triticum sp.
ccacgcgtccggctagttcatgtaagcttcctcctctccacctacaaagcaggagctcagtctcaggtgctgggatagctctgctagttcgtcgactttggaaatggccaccaactcgaggtcgaactccagggcgaccttctccagcgagatccacgacatcggcacggtgcagaactccaccacgcccagcatggtgtactacaccgagcggtccatcgccgactacttccctcctcacctcctcaagaaggtggtgtcggaggtggtgtcgacgttcctgctggtgttcgtgacgtgcggggcggcggcgatcagcgcccacgacgtcacgcgcatatcgcagctcggccagtcggtcgccggcgggctcatcgtcgtcgtcatgatctatgccgtcggccacatctccggcgcgcacatgaaccccgccgtcaccctcgccttcgccatattccgccatttcccctggattcaggtcccgttctactgggcggcgcagttcacgggcgcgatctgcgcgtccttcgtgctcaaggcggtgctccaccccatcaccgtgatcggcaccaccgagccggtcgggccgcactggcacgcgctggtcatcgaggtcgtcgtcaccttcaacatgatgttcgtcaccctcgccgtcgccacggacactagagcggtgggtgagttggctgggttggctgtcggttcctccgtttgcattacctccatcttcgcaggggcggtgtcaggtggatcgatgaacccggcgaggacgctgggcccggcgctggccagcaaccgctaccccggcctctggctctacttcctgggccccgtcctcggcacgctctccggggcctggacctacacctacatccgcttcgaggagccgcccaaggacgggccccagaagctctcctccttcaagctccggcggctgcagagccagtccgtggccgccgacgacgacgagctcgaccacatccccgtctgatcctgccgccgccggaccggatggatctcccactcccagtgtgtgtccttggtagttgttatgtgtgtgcgggcgtgtgtcacttgtaactcagtggttggttggtcgatgcatgcaggggagtacgcgcgtgcgcgcatgtgtatgtttgttcgcccgcgcatgtcgcagtagatttgctccctcgctctgttcatgtgtgcaagcgactgagcttggggtcctgattttgggggcgtatcagtccgcactctgcagtcgcacgcgtgccaaagtgtatctctcctgcaattgcagtacataaaactcccgtggctttgcttctcgaaagaacatgtgttgacgttcc

SEQ ID NO: 3 - DNA, 897 bp, Oryza sativa
atggccagcaacaactcgagaacaaactccagggcgaactactccaacgagatccacgatctctccacggtgcagaacggcaccatgcctaccatgtactacggcgagaaggccatcgccgacttcttccctcctcacctcctcaagaaggtcgtgtcggaggtggtggccacgttcctgctggtgttcatgacgtgtggggcggcagggatcagcggcagcgacctgtctcgcatatcgcagctgggacagtcgatcgccggtggcctcatcgtgacggtgatgatctacgccgtcggccacatctccggcgcccacatgaaccccgccgtgacgctcgcgttcgccgtgttcaggcatttcccctggattcaggttccgttctactgggcggcgcagttcaccggagcgatatgcgcgtcgttcgtgctcaaggcggtgatccacccggtggatgtgatcggaaccaccacgcccgtggggccgcactggcactcgctcgtcgtcgaggtcatcgtgacgttcaacatgatgttcgtcacgctcgccgtcgccacggacacgagagcggtgggtgagttggccgggttggcggttggttccgcggtttgcattacgtccatcttcgcaggggcaatttcaggtggatcgatgaacccggcaaggacgctggggccggcgctggcgagcaacaagttcgacggcctgtggatctacttcctgggcccagtcatgggcacgctctcgggagcatggacctacaccttcatccgcttcgaggacacccccaaggaaggctcctcccagaagctctcctccttcaagctgcgccgcttgcggagccagcagtccatcgccgccgacgacgtcgacgagatggagaacatccaagtgtga

SEQ ID NO: 4 - DNA, 888 bp, Triticum sp.
atggccaccaactcgaggtcgaactccagggcgaccttctccagcgagatccacgacatcggcacggtgcagaactccaccacgcccagcatggtgtactacaccgagcggtccatcgccgactacttccctcctcacctcctcaagaaggtggtgtcggaggtggtgtcgacgttcctgctggtgttcgtgacgtgcggggcggcggcgatcagcgcccacgacgtcacgcgcatatcgcagctcggccagtcggtcgccggcgggctcatcgtcgtcgtcatgatctatgccgtcggccacatctccggcgcgcacatgaaccccgccgtcaccctcgccttcgccatattccgccatttcccctggattcaggtcccgttctactgggcggcgcagttcacgggcgcgatctgcgcgtccttcgtgctcaaggcggtgctccaccccatcaccgtgatcggcaccaccgagccggtcgggccgcactggcacgcgctggtcatcgaggtcgtcgtcaccttcaacatgatgttcgtcaccctcgccgtcgccacggacactagagcggtgggtgagttggctgggttggctgtcggttcctccgtttgcattacctccatcttcgcaggggcggtgtcaggtggatcgatgaacccggcgaggacgctgggcccggcgctggccagcaaccgctaccccggcctctggctctacttcctgggccccgtcctcggcacgctctccggggcctggacctacacctacatccgcttcgaggagccgcccaaggacgggccccagaagctctcctccttcaagctccggcggctgcagagccagtccgtggccgccgacgacgacgagctcgaccacatccccgtctga

SEQ ID NO: 5 - protein, 298 aa, Oryza sativa
MASNNSRTNSRANYSNEIHDLSTVQNGTMPTMYYGEKAIADFFPPHLLKKVVSEVVATFLLVFMTCGAAGISGSDLSRISQLGQSIAGGLIVTVMIYAVGHISGAHMNPAVTLAFAVFRHFPWIQVPFYWAAQFTGAICASFVLKAVIHPVDVIGTTTPVGPHWHSLVVEVIVTFNMMFVTLAVATDTRAVGELAGLAVGSAVCITSIFAGAISGGSMNPARTLGPALASNKFDGLWIYFLGPVMGTLSGAWTYTFIRFEDTPKEGSSQKLSSFKLRRLRSQQSIAADDVDEMENIQV

SEQ ID NO: 6 - protein, 295 aa, Triticum sp
MATNSRSNSRATFSSEIHDIGTVQNSTTPSMVYYTERSIADYFPPHLLKKVVSEVVSTFLLVFVTCGAAAISAHDVTRISQLGQSVAGGLIVVVMIYAVGHISGAHMNPAVTLAFAIFRHFPWIQVPFYWAAQFTGAICASFVLKAVLHPITVIGTTEPVGPHWHALVIEVVVTFNMMFVTLAVATDTRAVGELAGLAVGSSVCITSIFAGAVSGGSMNPARTLGPALASNRYPGLWLYFLGPVLGTLSGAWTYTYIRFEEPPKDGPQKLSSFKLRRLQSQSVAADDDELDHIPV

SEQ ID NO: 7 - DNA, 23 bp, Artificial Sequence
  Primer
tccctcctcacctcctcaagaag

SEQ ID NO: 8 - DNA, 23 bp, Artificial Sequence
  Primer
agcttgaaggaggagagcttctg

SEQ ID NO: 9 - DNA, 659 bp, Triticum sp.
cctcctcaagaaggtggtgtcggaggtggtgtcgacgttcctgctggtgttcgtgacgtgcggggcggcggcgatcagcgcccacgacgtcacgcgcatatcgcagctcggccagtcggtcgccggcgggctcatcgtcgtcgtcatgatctatgccgtcgggcacatctccggcgcgcacatgaaccccgccgtcaccctcgccttcgccatattccgccatttcccctggattcaggtcccgttctactgggcggcgcagttcaccggcgcgatctgcgcgtccttcgtgctcaaggcggtgctccaccccatcaccgtgatcggcaccaccgagccggtcgggccgcactggcacgcgctggtcatcgaggtcgtcgtcaccttcaacatgatgttcgtcaccctcgccgtcgccacggacaccagagcggtgggtgagttggctgggttggctgtcggttcctccgtttgcattacctccatcttcgcaggggcggtgtcaggtggatcgatgaacccggcgaggacgctgggcccggcgctggccagcaaccgctaccccggcctctggctctacttcctgggccccgtcctcggctacgctcacgcggggcctggacctacacctacatccgcttcgaggacccgcccaagga

SEQ ID NO: 10 - DNA, 23 bp, Artificial Sequence
  Primer
cgaagatggacgtaatgcaaacc

SEQ ID NO: 11 - DNA, 20 bp, Artificial Sequence
  Primer
cgcccagtagaacggaacct

SEQ ID NO: 12 - DNA, 674 bp, Triticum sp.
tccctcctcacctcctcaagaaggtggtgtcggaggtggtgtcgacgttcctgctggtgttcgtgacgtgcggggcggcggcgatcagcgcccacgacgtcacgcgcatatcgcagctcggccagtcggtcgccggcgtggatcatcgtcgtcgtcatgatctatgccgtcgggcacatctccggcgcgcacatgaaccccgccgtcaccctcgccttcgccatattccgccatttcccctggattcaggtcccgttctactgggcggcgcagttcaccggcgcgatctgcgcgtccttcgtgctcaaggcggtgctccaccccatcaccgtgatcggcaccaccgagccggtcgggccgcactggcacgcgctggtcatcgaggtcgtcgtcaccttcaacatgatgttcgtcaccctcgccgtcgccacggacaccagagcggtgggtgagttggctgggttggctgtcggttcctccgtttgcattacctccatcttcgcaggggcggtgtcaggtggatcgatgaacccggcgaggacgctgggcccggcgctggccagcaaccgctaccccggcctctggctctacttcctgggccccgtcctcggcacgctcaccggggcctggacctacacctacatccgcttcgaggacccgcccaaggacggccc

SEQ ID NO: 13 - DNA, 615 bp, Hordeum vulgare
ggcgacgttcctgctggtgttcgtgacgtgcggggcggcgtccatctacggcgccgacgtgacgcgcgtctcgcagctgggccagtccgtcgtcgggggcctcatcgtcaccgtcatgatctacgccaccggacacatctccggcgcgcacatgaaccccgccgtcaccctctccttcgcctgcttccggcatttcccctggattcaggtgccgttctactgggcggcgcagttcacgggggcgatgtgcgcggcgttcgtgctgcgggcggtgctgcacccgatcacggtgctggggacgaccacgcccacggggccgcactggcacgcgctcgtcatcgagatcatcgtcaccttcaacatgatgttcatcacctgcgccgtcgccacggactcgagagcggtgggtgagttggcagggttagcagttggttccgcggtttgcattacgtccatcttcgcagggcctgtgtcaggaggatcgatgaacccggcgaggaccctggcgccggcggtggccagcggcgtctacaccggcctgtggatctactttctcggccccgtcatcggcacgctctccggcgcgtgggtctacacctacatccgcttcgagga

SEQ ID NO: 14 - DNA, 482 bp, Avena sativa
ccctcctcaagaagatggtgtcggaggtggtgtcgacgttcctgctggtgttcatgacttgcggagctgcggcgatcagcgccagcgaccccacgcgcatatcgcagctgggacagtcggtcgcaggcggtctcatcgtgacggtgatgatctactccgtcggacacatctccggcgcgcacatgaaccccgccgtcacactctccttcgccgtgttccggcatttcccatggattcaggtccctttctactgggcgtcccagttcacgggtgcgatctgtgcgtccttcgtgctcaaggcggtgctccaccccatcaccgtgatcggcaccaccgtgccgcacggcccgcactggcactcgctcgtcatcgaggtcgtagtcaccttcaacatgatgttcgtcacgctcgccgtcgcaacggacaacagggcggtgggtgagttggccgggttagccgtcggttcctcggtttgcattacg

SEQ ID NO: 15 - DNA, 824 bp, Equisetum sp.
atggccaccaactcgaggtcgaactccagggcgaccttcgccagcgagatccacgacatcggcacggtgcagaactccaccacgcccagcatggtgtactacaccgagcggtctatcgccgactacttccctcctcacctcctcaagaaggtggtgtcggaggtggtgtcgacgttcctgctggtgttcgtgacgtgcggggcggcggcgatcagcgcccacgacgtgacgcgcatatcgcagctcggccaggcggtcgccggcgggctcatcgtcgtcgtgatgatctatgccgtcggccacatctctggcgcgcacatgaaccccgccgtcaccctcaccttcgccatcttccgccatttcccctggattcaggtcccgttctactgggcggcgcagttcacgggcgcgatctgcgcgtccttcgtgctcaaggcggtgctccaccccatcaccgtgatcggcaccaccgagccggtcgggccgcactggcacgcgctggtcatcgaggtcgtcgtcaccttcaacatgatgttcgtcaccctcgaagtcgccacggacacgagagcggtgggtgagttggctgggttggctgtcggttcctccgtttgcattacctccatcttcgcaggggcggtgtcaggtggatcgatgaacccggcgaggacgctgggcccggcgctggccagcaaccgctaccccggcctctggctctacttcctgggccccgtcctcggcacgctcagcggggcctggacctacacctacatccgcttcgaggacccgcccaaggacggcccccagaagctctcctccttcaagct

SEQ ID NO: 16 - DNA, 40 bp, Artificial Sequence
  Primer
ggaatcagtcagtaattggaggtttttttttttttttttt

SEQ ID NO: 17 - DNA, 22 bp, Artificial Sequence
  Primer
ggaatcagtcagtaattggagg

SEQ ID NO: 18 - DNA, 18 bp, Artificial Sequence
  Primer
tcctcgaagcggatgtag

SEQ ID NO: 19 - DNA, 19 bp, Artificial Sequence
  Primer
cctgcgaagatggaggtaa

SEQ ID NO: 20 - DNA, 19 bp, Artificial Sequence
  Primer
cgagggtgacgaacatcat

SEQ ID NO: 21 - DNA, 919 bp, Triticum sp
tttttttttttttttttttcgagaagcgagctaagctagttcatgtaagcttcttcctctccacctacaaagcaggagctcagtctcaggtgctgggatagctctgatagttcgtcttggaaaatggccaccaactcgaggtcgaactccagggcgaccttctccagcgagatccacgacatcggcacggtgcagaactccaccacgcccagcatggtgtactacaccgagcggtccatcgccgactacttccctcctcacctcctcaagaaggtggtgtcggaggtggtgtcgacgttcctgctggtgttcgtgacgtgcggggcggcggcgatcagcgcccacgacgtgacgcgcatatcgcagctcggccagtcggtcgccggcgggctcatcgtcgtcgtgatgatctatgccgtcggccacatctctggcgcgcacatgaaccccgccgtcaccctcgccttcgccatcttccgccatttcccctggattcaggtcccgttctactgggcggcgcagttcacgggcgcgatctgcgcgtccttcgtgctcaaggcggtgctccaccccatcaccgtgatcggcaccaccgagccggtcgggccgcactggcacgcgctggtcatcgaggtcgtcgtcaccttcaacatgatgttcgtcaccctcgccgtcgccacggacactagagcggtgggtgagttggctgggttggctgtcggttcctccgtttgcattacctccatcttcgcaggggcggtgtcaggtggatcgatgaacccggcgaggacgctgggcccggcgctggccagcaaccgctaccccggcctctggctctacttcctgggccccgtcctcggctacgctcacgcggggcctggacctacacctacatccgcttcgaggacccgcccaagga

SEQ ID NO: 22 - DNA, 694 bp, Equisetum sp.
ttggaatcagtcagtaattggaggtttttttttttcttttccgagaagcgagctgagctagttcatgtaagcttcttcctctccacctacaaagcaggagcttagtctcaggtgctgggatagctctgatagttcgtcttggaaaatggccaccaactcgaggtcgaactccagggcgaccttcgccagcgagatccacgacatcggcacggtgcagaactccaccacgcccagcatggtgtactacaccgagcggtctatcgccgactacttccctcctcacctcctcaagaaggtggtgtcggaggtggtgtcgacgttcctgctggtgttcgtgacgtgcggggcggcggcgatcagcgcccacgacgtgacgcgcatatcgcagctcggccaggcggtcgccggcgggctcatcgtcgtcgtgatgatctatgccgtcggccacatctctggcgcgcacatgaaccccgccgtcaccctcaccttcgccatcttccgccatttcccctggattcaggtcccgttctactgggcggcgcagttcacgggcgcgatctgcgcgtccttcgtgctcaaggcggtgctccaccccatcaccgtgatcggcaccaccgagccggtcgggccgcactggcacgcgctggtcatcgaggtcgtcgtcaccttcaacatgatgttcgtcaccctcgaa

SEQ ID NO: 23 - DNA, 2085 bp, Oryza sativa
gttcagcagcgcatgcactagcagcttagctactgcgcgcgtaccagagagagatcatcagctcaagtagctaagctagctataatctgctagctagctcgatcagacacttaattacctgctaggtggtggtcgatcgaagaagaagaagatgagtgagcttgcgtcggcgcccaaggtggcgcttggatcgatcgcgttcgcggtgttctggatgatggcggtgttcccgtcggtgccgttcctgccgatcgggcggacggcggggtcgctgctgagcgcggtgctgatggtgatattccacgtgatcagccccgacgacgcgtacgcctccatcgacctcccaatcctgggcctcctcttcgccaccatggtggtgggcagctacctccggaacgccgggatgttcaagcacctggggcgtctgctggcgtggaagagccagggcgggcgcgacctcatgtgccgcgtctgcgtcgtcaccgccctcgccagcgccctcttcaccaacgacacctgctgcgtcgtcctcaccgagttcgtcctcgagctcgccgccgagcgcaacctccccgccaagcccttcctcctcgccctcgcctccagcgccaacatcggctccgccgccacccccatcggcaacccccagaacctggtcatcgccttcaatagcaagatcaccttccccaagttcctcatgggaatcctcccggccatgctcgtcgggatggccgtcaacatggtcatgctgctctgcatgtactggagggagctgggcggaggggccgagctcagcgtcgacggcaagcagatggaggcggtggaggaaggcaggtcgccggcgtcggccaagagcacgccgcagctgaacggcaacggcaacacgatgatgtcgctggagatgtcggagaacataacgaccaagcacccatggttcatgcagtgcacggaggcgcggcggaagctgttcctcaagagcttcgcgtacgtggtgacggtggggatggtggtggcctacatggtggggctcaacatgtcgtggacggccatcaccacggcgctggcgctggtggtggtcgacttccgcgacgccgagccgtgcctggacaccgtgtcctactcgctgctcgtcttcttctccgggatgttcatcaccgtcagcggcttcaacaagacgggcctcccgggagccatctgggacttcatggccccctactccaaggtcaacagcgtcggcggcatctccgtcctctccgtcatcatcctcctcctctccaacctcgcatcaaacgtaccaacggtgcttcttatgggtgatgaggtggcgaaggcggcggcgctgatatcgccggcggcggtgacgacgtcgtggctgctgctggcgtgggtgagcacggtggcggggaacctgtcgctgctggggtcggcggcgaacctgatagtgtgcgagcaggcgaggagggcgcccaggaacgcctacgacctcaccttctggcagcacatcgtcttcggcgtcccatccaccctcatcgtcaccgccgtcggcatacccctcatcggcaagatctgatctcatctcatcgacccatccaaattaattaattatgagatcgacaaacatccaagcttgctaggctcgtcgtcgtcgtcgtcgaccaccgtaccatatatatgcatgcatgccacgcacgtatatatatgctcctcaacctcaagtcaattaattaagagaatggatgaatgaatcaatgttgtgtatgatttcctttttgtttttgttttacccaccgtatatgtgctgtgtggtgtatggccggcgagcttgaaattgaattggccttgcttgcatgcatgcatggtgatcagatcagctcagctagctagctaaatcttgcttaattttatcaagtgtcaatcatgatgagaagagagggaactagatggagctagctagctagcaacacatgcagtgtgtgctaagcagtgcactctgcatgcttaaatttgcttgcttaattcaacatcgtattacatgtatgttgatatgtgcatgttcctcaaaatacatggaaaaaaaatgtttct

SEQ ID NO: 24 - DNA, 1205 bp, Triticum sp.
  misc_feature (823)..(823): n is a, c, g, or t
acccgctatttacgattaggcctattcaggtgacattatagaacaagtttgtacaaaaaagcaggctggtaccggtccggaattcccgggatatcgtcgacccacgcgtccgcaccggtgcatgcacactgccttcgaggtgcagcgccactaaccaagcagagcactaaccagccaccttaactagatagctccatctcccaacaccggcggccagccatggcgctcgcgtctctccccaaggtggtgctcggctccatcgccttcgccgtcttctggatgatggcggtgttcccgtcggtgcccttcctgcccatcggccgcacggcgggctcgctgctctccgccgtgctcatgatcgtcttccacgtgatcagccccgacgacgcctacgcctccatcgacctccccatcctcggcctgctcttctccaccatggtcgtcggcggctacctcaagaacgccggcatgttcaagcacctcggcaccctcctcgcctggaagagccagggcggccgcgacctgctctgccgcgtctgcgtcgtcaccgcgctcgcctccgcgctcttcaccaacgacacctgctgcgtcgtgctcaccgagttcgtgctcgagctcgccgccgagcggaacctcccggccaagcccttcctcctggccctcgcctccagcgccaacatcggctccagcgccacccccatcggcaacccgcagaacctggtcatcgccttcaacagcaagatctccttcccaaaggttcctcatcggcatcctgccggccatgctcgccaggcatggaccgtcaacatggtcatgctgctcntgcatgtactggaaggacctcgagggtcgtggcccccgacgcggccggcaagcagatgtcggtcgtcgaggagggcggccgctcgccgtccgtggcatcgctcaagagcccgcacccgttcaacggcaccacggccgacgacggcaacgagtcgatgatggaggagaacatctcgaccaagcacccgtggttcatgcagtgcacggagcaccgacgcaagctgttcctcaagagcttcgcctacatcgtgacgctgggcatggtggtagcatacatggccgggctcaacatgtcgtggacggccatcaccaccgccatcgcgctggtcgtcgtcgacttccgggacgccgagccgtgcctcgtcaaggtctcctactcgctg

SEQ ID NO: 25 - DNA, 22 bp, Artificial Sequence
  Primer
tgttctggatgatggcggtgtt

SEQ ID NO: 26 - DNA, 24 bp, Artificial Sequence
  Primer
tgaacatcccggagaagaagacga

SEQ ID NO: 27 - DNA, 850 bp, Triticum sp.
  misc_feature (541)..(541): n is a, c, g, or t
atgatggcggtgttcccgtcggtgcccttcctgcccatcggccgcacggcgggctcgctgctctccgccgtgctcatgatcgtcttccacgtgatcagccccgacgacgcctacgcctccatcgacctccccatcctcggcctgctcttctccaccatggtcgtcggcggctacctcaagaacgccggcatgttcaagcacctcggcaccctcctcgcctggaagagccagggcggccgcgacctgctctgccgcgtctgcgtcgtcaccgcgctcgcctccgcgctcttcaccaacgacacctgctgcgtcgtgctcaccgagttcgtgctcgagctcgccgccgagcggaacctcccggccaagcccttcctcctggccctcgcctccagcgccaacatcggctccagcgccacccccatcggcaacccgcagaacctggtcatcgccttcaacagcaagatctcgtttccaaagtttttgatcggcatcctgccggccatgctcgccggcatggccgtcaacatggtcatgctgctcntgcatgtactggaaggacctggagggcgtggcccccgacgcggccggcaagcagatgtcggtcgtcgaggaggggggccgctcgccgtccgtggcatcgctcaagagcccgcacccgttcaacggcaccacggccgacgacgggaacgagtcgatgatggaggagaacatctcgaccaagcacccgtggttcatgcagtgcacggagcaccggcgcaagctgttcctcaagagcttcgcctacatcgtgacgctgggcatggtggtagcatacatggccgggctcaacatgtcgtggacggccatcacc

SEQ ID NO: 28 - DNA, 1419 bp, Oryza sativa
atgagtgagcttgcgtcggcgcccaaggtggcgcttggatcgatcgcgttcgcggtgttctggatgatggcggtgttcccgtcggtgccgttcctgccgatcgggcggacggcggggtcgctgctgagcgcggtgctgatggtgatattccacgtgatcagccccgacgacgcgtacgcctccatcgacctcccaatcctgggcctcctcttcgccaccatggtggtgggcagctacctccggaacgccgggatgttcaagcacctggggcgtctgctggcgtggaagagccagggcgggcgcgacctcatgtgccgcgtctgcgtcgtcaccgccctcgccagcgccctcttcaccaacgacacctgctgcgtcgtcctcaccgagttcgtcctcgagctcgccgccgagcgcaacctccccgccaagcccttcctcctcgccctcgcctccagcgccaacatcggctccgccgccacccccatcggcaacccccagaacctggtcatcgccttcaatagcaagatcaccttccccaagttcctcatgggaatcctcccggccatgctcgtcgggatggccgtcaacatggtcatgctgctctgcatgtactggagggagctgggcggaggggccgagctcagcgtcgacggcaagcagatggaggcggtggaggaaggcaggtcgccggcgtcggccaagagcacgccgcagctgaacggcaacggcaacacgatgatgtcgctggagatgtcggagaacataacgaccaagcacccatggttcatgcagtgcacggaggcgcggcggaagctgttcctcaagagcttcgcgtacgtggtgacggtggggatggtggtggcctacatggtggggctcaacatgtcgtggacggccatcaccacggcgctggcgctggtggtggtcgacttccgcgacgccgagccgtgcctggacaccgtgtcctactcgctgctcgtcttcttctccgggatgttcatcaccgtcagcggcttcaacaagacgggcctcccgggagccatctgggacttcatggccccctactccaaggtcaacagcgtcggcggcatctccgtcctctccgtcatcatcctcctcctctccaacctcgcatcaaacgtaccaacggtgcttcttatgggtgatgaggtggcgaaggcggcggcgctgatatcgccggcggcggtgacgacgtcgtggctgctgctggcgtgggtgagcacggtggcggggaacctgtcgctgctggggtcggcggcgaacctgatagtgtgcgagcaggcgaggagggcgcccaggaacgcctacgacctcaccttctggcagcacatcgtcttcggcgtcccatccaccctcatcgtcaccgccgtcggcatacccctcatcggcaagatctga

SEQ ID NO: 29 - DNA, 989 bp, Triticum sp.
  misc_feature (607)..(607): n is a, c, g, or t
atgatggcgctcgcgtctctccccaaggtggtgctcggctccatcgccttcgccgtcttctggatgatggcggtgttcccgtcggtgcccttcctgcccatcggccgcacggcgggctcgctgctctccgccgtgctcatgatcgtcttccacgtgatcagccccgacgacgcctacgcctccatcgacctccccatcctcggcctgctcttctccaccatggtcgtcggcggctacctcaagaacgccggcatgttcaagcacctcggcaccctcctcgcctggaagagccagggcggccgcgacctgctctgccgcgtctgcgtcgtcaccgcgctcgcctccgcgctcttcaccaacgacacctgctgcgtcgtgctcaccgagttcgtgctcgagctcgccgccgagcggaacctcccggccaagcccttcctcctggccctcgcctccagcgccaacatcggctccagcgccacccccatcggcaacccgcagaacctggtcatcgccttcaacagcaagatctccttcccaaaggttcctcatcggcatcctgccggccatgctcgccaggcatggaccgtcaacatggtcatgctgctcntgcatgtactggaaggacctcgagggtcgtggcccccgacgcggccggcaagcagatgtcggtcgtcgaggagggcggccgctcgccgtccgtggcatcgctcaagagcccgcacccgttcaacggcaccacggccgacgacggcaacgagtcgatgatggaggagaacatctcgaccaagcacccgtggttcatgcagtgcacggagcaccgacgcaagctgttcctcaagagcttcgcctacatcgtgacgctgggcatggtggtagcatacatggccgggctcaacatgtcgtggacggccatcaccaccgccatcgcgctggtcgtcgtcgacttccgggacgccgagccgtgcctcgtcaaggtctcctactcgctg

SEQ ID NO: 30 - protein, 472 aa, Oryza sativa
MSELASAPKVALGSIAFAVFWMMAVFPSVPFLPIGRTAGSLLSAVLMVIFHVISPDDAYASIDLPILGLLFATMVVGSYLRNAGMFKHLGRLLAWKSQGGRDLMCRVCVVTALASALFTNDTCCVVLTEFVLELAAERNLPAKPFLLALASSANIGSAATPIGNPQNLVIAFNSKITFPKFLMGILPAMLVGMAVNMVMLLCMYWRELGGGAELSVDGKQMEAVEEGRSPASAKSTPQLNGNGNTMMSLEMSENITTKHPWFMQCTEARRKLFLKSFAYVVTVGMVVAYMVGLNMSWTAITTALALVVVDFRDAEPCLDTVSYSLLVFFSGMFITVSGFNKTGLPGAIWDFMAPYSKVNSVGGISVLSVIILLLSNLASNVPTVLLMGDEVAKAAALISPAAVTTSWLLLAWVSTVAGNLSLLGSAANLIVCEQARRAPRNAYDLTFWQHIVFGVPSTLIVTAVGIPLIGKI

SEQ ID NO: 31 - protein, 253 aa, Triticum sp.
  misc_feature (202)..(202): Xaa can be any naturally occurring amino acid
MALASLPKVVLGSIAFAVFWMMAVFPSVPFLPIGRTAGSLLSAVLMIVFHVISPDDAYASIDLPILGLLFSTMVVGGYLKNAGMFKHLGTLLAWKSQGGRDLLCRVCVVTALASALFTNDTCCVVLTEFVLELAAERNLPAKPFLLALASSANIGSSATPIGNPQNLVIAFNSKISFPKVPHRHPAGHARQAWTVNMVMLLXHVLEGPRGSWPPTRPASRCRSSRRAAARRPWHRSRARTRSTAPRPTTATSR

SEQ ID NO: 32 - DNA, 1311 bp, Triticum sp
tttttttttttttttttttcgagaagcgagctaagctagttcatgtaagcttcttcctctccacctacaaagcaggagctcagtctcaggtgctgggatagctctgatagttcgtcttggaaaatggccaccaactcgaggtcgaactccagggcgaccttctccagcgagatccacgacatcggcacggtgcagaactccaccacgcccagcatggtgtactacaccgagcggtccatcgccgactacttccctcctcacctcctcaagaaggtggtgtcggaggtggtgtcgacgttcctgctggtgttcgtgacgtgcggggcggcggcgatcagcgcccacgacgtcacgcgcatatcgcagctcggccagtcggtcgccggcgggctcatcgtcgtcgtcatgatctacgccgtcggccacatctccggcgcgcacatgaaccccgccgtcaccctcgccttcgccatattccgccatttcccctggattcaggtcccgttctactgggcggcgcagttcacgggcgcgatctgcgcgtccttcgtgctcaaggccgtgctccaccccatcaccgtgatcggcaccaccgagccggtcgggccgcactggcacgcgctggtcatcgaggtcgtcgtcaccttcaacatgatgttcgtcaccctcgccgtcgccacggacaccagagcggtgggtgagttggctgggttggctgtcggttcctccgtttgcattacctccatcttcgcaggggcggtgtcaggtggatcgatgaacccggcgaggacgctgggcccggcgctggccagcaaccgctaccccggcctctggctctacttcctcggccccgtcctcggctacgctcacgcggggcctggacctacacctacatccgcttcgaggacccgcccaaaggacggcccccagaagctctcctccttcaagctccgccggctgcagagccagtccgtcgccgccgacgacgacgagctcgaccacatccccgtctgatcctgccgccgcacgccgggccggacggatctcccgctcccagtgtgtgtccttggtagttgttgttatgtgtgtgcgtgcgtgtgtcacttgtaactcagtggttggtcgatgcatgcaggggagtacgcgcgtgcgcgcatgtgtatgtttgtccgcccgcgcatgtcgcagtagatttgctccctcgctctgttcatgtgtgcaggcgactgagcttgggggcctggtttgggggcgcatcagtccgcactctgcagcagtcgcacgcgtgccaaagtgtatctctcctgcaat

SEQ ID NO: 33 - DNA, 334 bp, Hordeum vulgare
  misc_feature (12)..(12): n is a, c, g, or t
cacggacactanagcggttggtgagttggctgggttggctgtcggttcctccgtttgcattacctccatcttcgcaggggcggtgtcaggtggatcgatgaacccggcgaggacgctgggcccggcgctggcgagcaaccgctaccctggcctctggctctacttcctgggacccgtccttggcacgctcagcggggcctggacctacacctacatccgcttcgaggacccgcccaaggacgcgccccagaagctctcctccttcaagctccggcggctgcagagccagtccgtggccgccgacgacgacgagctcgaccacatccccgtctga

SEQ ID NO: 34 - protein, 204 aa, Hordeum vulgare
ATFLLVFVTCGAASIYGADVTRVSQLGQSVVGGLIVTVMIYATGHISGAHMNPAVTLSFACFRHFPWIQVPFYWAAQFTGAMCAAFVLRAVLHPITVLGTTTPTGPHWHALVIEIIVTFNMMFITCAVATDSRAVGELAGLAVGSAVCITSIFAGPVSGGSMNPARTLAPAVASGVYTGLWIYFLGPVIGTLSGAWVYTYIRFE

SEQ ID NO: 35 - protein, 160 aa, Avena sativa
LLKKMVSEVVSTFLLVFMTCGAAAISASDPTRISQLGQSVAGGLIVTVMIYSVGHISGAHMNPAVTLSFAVFRHFPWIQVPFYWASQFTGAICASFVLKAVLHPITVIGTTVPHGPHWHSLVIEVVVTFNMMFVTLAVATDNRAVGELAGLAVGSSVCIT

SEQ ID NO: 36 - protein, 274 aa, Equisetum sp.
MATNSRSNSRATFASEIHDIGTVQNSTTPSMVYYTERSIADYFPPHLLKKVVSEVVSTFLLVFVTCGAAAISAHDVTRISQLGQAVAGGLIVVVMIYAVGHISGAHMNPAVTLTFAIFRHFPWIQVPFYWAAQFTGAICASFVLKAVLHPITVIGTTEPVGPHWHALVIEVVVTFNMMFVTLEVATDTRAVGELAGLAVGSSVCITSIFAGAVSGGSMNPARTLGPALASNRYPGLWLYFLGPVLGTLSGAWTYTYIRFEDPPKDGPQKLSSFK

SEQ ID NO: 37 - protein, 296 aa, Triticum sp.
MATNSRSNSRATFSSEIHDIGTVQNSTTPSMVYYTERSIADYFPPHLLKKVVSEVVSTFLLVFVTCGAAAISAHDVTRISQLGQSVAGGLIVVVMIYAVGHISGAHMNPAVTLAFAIFRHFPWIQVPFYWAAQFTGAICASFVLKAVLHPITVIGTTEPVGPHWHALVIEVVVTFNMMFVTLAVATDTRAVGELAGLAVGSSVCITSIFAGAVSGGSMNPARTLGPALASNRYPGLWLYFLGPVLGYAHAGPGPTPTSASRTRPKDGPQKLSSFKLRRLQSQSVAADDDELDHIPV

SEQ ID NO: 38 - protein, 110 aa, Hordeum vulgare
  misc_feature (4)..(4): Xaa can be any naturally occurring amino acid
TDTXAVGELAGLAVGSSVCITSIFAGAVSGGSMNPARTLGPALASNRYPGLWLYFLGPVLGTLSGAWTYTYIRFEDPPKDAPQKLSSFKLRRLQSQSVAADDDELDHIPV

SEQ ID NO: 39 - DNA, 897 bp, Artificial sequence
  Mutated rice SIIT1
atggccagcaacaactcgagaacaaactccagggcgaactactccaacgagatccacgatctctccacggtgcagaacggcaccatgcctaccatgtactacggcgagaaggccatcgccgacttcttccctcctcacctcctcaagaaggtcgtgtcggaggtggtggccacgttcctgctggtgttcatgacgtgtggggcggcagggatcagcggcagcgacctgtctcgcatatcgcagctgggacagtcgatcgccggtggcctcatcgtgacggtgatgatctacgccgtcggccacatctccggcgcccacatgaaccccgccgtgacgctcgcgttcgccgtgttcaggcatttcccctggattcaggttccgttctactgggcgacgcagttcaccggagcgatatgcgcgtcgttcgtgctcaaggcggtgatccacccggtggatgtgatcggaaccaccacgcccgtggggccgcactggcactcgctcgtcgtcgaggtcatcgtgacgttcaacatgatgttcgtcacgctcgccgtcgccacggacacgagagcggtgggtgagttggccgggttggcggttggttccgcggtttgcattacgtccatcttcgcaggggcaatttcaggtggatcgatgaacccggcaaggacgctggggccggcgctggcgagcaacaagttcgacggcctgtggatctacttcctgggcccagtcatgggcacgctctcgggagcatggacctacaccttcatccgcttcgaggacacccccaaggaaggctcctcccagaagctctcctccttcaagctgcgccgcttgcggagccagcagtccatcgccgccgacgacgtcgacgagatggagaacatccaagtgtga

SEQ ID NO: 40 - protein, 298 aa, Artificial Sequence
  Mutated rice SIIT1
MASNNSRTNSRANYSNEIHDLSTVQNGTMPTMYYGEKAIADFFPPHLLKKVVSEVVATFLLVFMTCGAAGISGSDLSRISQLGQSIAGGLIVTVMIYAVGHISGAHMNPAVTLAFAVFRHFPWIQVPFYWATQFTGAICASFVLKAVIHPVDVIGTTTPVGPHWHSLVVEVIVTFNMMFVTLAVATDTRAVGELAGLAVGSAVCITSIFAGAISGGSMNPARTLGPALASNKFDGLWIYFLGPVMGTLSGAWTYTFIRFEDTPKEGSSQKLSSFKLRRLRSQQSIAADDVDEMENIQV

SEQ ID NO: 41 - DNA, 37 bp, Artificial Sequence
  Primer
ggaattcatggccagcaacaactcgagaacaaactcc

SEQ ID NO: 42 - DNA, 39 bp, Artificial Sequence
  Primer
gtctagacctatcacacttggatgttctccatctcgtcg

SEQ ID NO: 43 - DNA, 22 bp, Artificial Sequnece
  Primer
caaccgttctactgggcgacgc

SEQ ID NO: 44 - DNA, 22 bp, Artificial Sequnence
  Primer
cgctccggtgaactgcgtcgcc

SEQ ID NO: 45 - DNA, 37 bp, Artificial Sequnece
  Primer
ggaattcatggccaccaactcgaggtcgaactccagg

SEQ ID NO: 46 - DNA, 39 bp, Artificial Sequnece
  Primer
gtctagacctatcagacggggatgtggtcgagctcgtcg

SEQ ID NO: 47 - DNA, 22 bp, Artificial Sequnece
  Primer
gtcccgttctactgggcgacgc

SEQ ID NO: 48 - DNA, 22 bp, Artificial Sequence
  Primer
cgcgcccgtgaactgcgtcgcc

SEQ ID NO: 49 - DNA, 3019 bp, Artifiical Sequence
  Expression vector Pol1
gggcgaattaattcgagctcggtacccagcttgcttgttctttttgcagaagctcagaataaacgctcaactttggcagatcaattccccggggatccgaattctctagagcaagcttgatctggttaccactaaaccagcctcaagaacacccgaatggagtctctaagctacataataccaacttacactttacaaaatgttgtcccccaaaatgtagccattcgtatctgctcctaataaaaagaaagtttcttcacattctaaaaaaaaaaaaaaaaaaaaaaaaaaaaaaaaacccccccccccccccccctgcaggcatgcaagctagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 50 - DNA, 891 bp, Artificial Sequence
  Mutated wheat SIIT1
atggccaccaactcgaggtcgaactccagggcgaccttctccagcgagatccacgacatcggcacggtgcagaactccaccacgcccagcatggtgtactacaccgagcggtccatcgccgactacttccctcctcacctcctcaagaaggtggtgtcggaggtggtgtcgacgttcctgctggtgttcgtgacgtgcggggcggcggcgatcagcgcccacgacgtcacgcgcatatcgcagctcggccagtcggtcgccggcgggctcatcgtcgtcgtcatgatctacgccgtcggccacatctccggcgcgcacatgaaccccgccgtcaccctcgccttcgccatattccgccatttcccctggattcaggtcccgttctactgggcgacgcagttcacgggcgcgatctgcgcgtccttcgtgctcaaggccgtgctccaccccatcaccgtgatcggcaccaccgagccggtcgggccgcactggcacgcgctggtcatcgaggtcgtcgtcaccttcaacatgatgttcgtcaccctcgccgtcgccacggacaccagagcggtgggtgagttggctgggttggctgtcggttcctccgtttgcattacctccatcttcgcaggggcggtgtcaggtggatcgatgaacccggcgaggacgctgggcccggcgctggccagcaaccgctaccccggcctctggctctacttcctcggccccgtcctcggctacgctcacgcggggcctggacctacacctacatccgcttcgaggacccgcccaaaggacggcccccagaagctctcctccttcaagctccgccggctgcagagccagtccgtcgccgccgacgacgacgagctcgaccacatccccgtctga

SEQ ID NO: 51 - protein, 296 aa, Artificial Sequence
  Mutated wheat SIIT1
MATNSRSNSRATFSSEIHDIGTVQNSTTPSMVYYTERSIADYFPPHLLKKVVSEVVSTFLLVFVTCGAAAISAHDVTRISQLGQSVAGGLIVVVMIYAVGHISGAHMNPAVTLAFAIFRHFPWIQVPFYWATQFTGAICASFVLKAVLHPITVIGTTEPVGPHWHALVIEVVVTFNMMFVTLAVATDTRAVGELAGLAVGSSVCITSIFAGAVSGGSMNPARTLGPALASNRYPGLWLYFLGPVLGYAHAGPGPTPTSASRTRPKDGPQKLSSFKLRRLQSQSVAADDDELDHIPV

SEQ ID NO: 52 - DNA, 894 bp, Sorghum sp
atgtcgaccaactcgaggtcgaactccagggccaacttcaacaacgagatccatgacatcggcacggtgcagaactccaccatgatgccccccacgtactacgaccgatcgctggcggacatcttccctccccacctcctcaagaaggtggtctcggaggtggtgtccacgttcttgctggtgttcgtgacgtgcggggcggcggggatctacggcagcgacaaggaccgcatatcgcagctgggacagtcggtcgccggcgggctcatcgtcacggtgatgatctacgccgtcggccacatctccggcgcgcacatgaaccccgccgtcacgctcgcgttcgccgtgttccgccatttcccctggattcaggtcccgttctactgggcggcgcagttcacgggcgccatctgcgcgtcgttcgtgctcaaggccgtgctgcaccccatctccgtgctgggctgcaccacgccgacggggccgcactggcactcgctcatcatcgagatcatcgtcaccttcaacatgatgttcgtcaccctcgccgtcgccacggacacgagagcggtgggtgagttggcggggttggcagttggttccgcggtttgcattacgtccatcttcgcaggggcagtgtctggcggatcgatgaacccggcgaggacgctggggccggcgctggcgagcaacctctacaccggactctggatctacttcttgggccccgtcctcggcacgctctcgggggcctggacctacacctacatccgcttcgaggaagcgcccagcacccacaaggacatgtcgcagaagctctcctccttcaagctccgccgcctgcagagccagtccgtcgccgcggaagacgacgagctcgaccacatccaagtgtga

SEQ ID NO: 53 - DNA, 888 bp, Zea mays
atgtcgaccaactcgaggtccaactccagggccaacttcaacaacgagatccatgacatcggcacggcgcagaactccagcatgccccccacgtactacgaccggtcgctggcggacatcttccctccgcacctcctcaagaaggtggtctcggaggtggtgtccacgttcctgctggtgttcgtcacgtgcggggcggcggggatctacggcagcgacaaggaccgcatctcgcagctggggcagtcggtcgccggcgggctcatcgtcaccgtcatgatctacgccgtcggacacatctcgggcgcgcacatgaaccccgccgtcacgctcgcgttcgccgtgttccgccatttcccctggatccaggtcccgttctactgggcggcgcagttcaccggcagcatctgcgcgtcgttcgtgctcaaggccgtgctgcaccccatcgccgtgctgggcaccaccacgccgacggggccgcactggcactcgctcgtcatcgagatcatcgtcaccttcaacatgatgttcgtcaccctcgccgtcgccacggacacgagagcggtgggtgagttggcggggttggcagttggttccgcggtttgcattacgtccatcttcgcaggggcagtgtcgggcggatcgatgaacccggcgaggacgctggggccggcgctggcgagcaacctctacaccggcctctggatctacttcctgggccccgtcctcggcacgctctccggggcctggacctacacctacatccgcttcgaggaggcgcccagccacaaggacatgtcgcagaagctctcctccttcaagctccgccgcctgcagagccagtccgtcgcggtcgacgacgacgagctcgaccacatccaagtgtga

SEQ ID NO: 54 - protein, 294 aa, Hordeum vulgare
MASNSRSNSRATFSSEIHDIGTVQNSTTPSMVYYTERSIADYFPPHLLKKVVSEVVSTFLLVFVTCGAAAISAHDVTRISQLGQSVAGGLIVVMIYAVGHISGAHMNPAVTLAFAIFRHFPWIQVPFYWAAQFTGAICASFVLKAVLHPITVIGTTEPVGPHWHALVIEVVVTFNMMFVTLAVATDTRAVGELAGLAVGSSVCITSIFAGAVSGGSMNPARTLGPALASNRYPGLWLYFLGPVLGTLSGAWTYTYIRFEDPPKDAPQKLSSFKLRRLQSQSVAADDDELDHIPV

SEQ ID NO: 55 - DNA, 897 bp, Oryza sativa
atggcatcgacgacagcgccgtcgaggaccaactctcgggtgaactactcgaacgagatccatgacctctccaccgtgcagagcgtctccgccgtccccagcgtctactaccccgagaaatccttcgccgacatcttccctcctaacctcctcaagaaggtgatatcggaggtggtggcgacgttcctgcttgtgttcgtgacgtgcggggcggcgtccatctacggcgaggacatgaagcgcatctcgcagctggggcagtcggtggtcggtggcctcatcgtcaccgtcatgatctacgccaccggccacatctccggcgcccacatgaacccggccgtcaccctctccttcgccttcttccggcatttcccctggattcaggtgccgttctactgggcggcgcagttcacgggggcgatgtgcgcggcgttcgtgctgcgggcggtgctgtacccgatcgaggtgttggggacgacgacgccgacggggccgcactggcacgccctcgtcatcgagatcgtcgtcaccttcaacatgatgttcgtcacctgcgccgttgccaccgactccagagcggtgggtgagttggcggggttagcagtcggttccgcggtttgcattacgtcgatcttcgcagggccggtgtcaggaggatcgatgaacccggcgaggacgctggcgccggcggtggccagcaacgtctacaccggcctctggatctacttcctcggccccgtcgtcggcaccctctccggcgcatgggtctacacctacatccgcttcgaggaggcccccgccgccgccggcggcgccgccccccagaagctctcctccttcaagctccgccgcttgcagagccagtccatggccgccgacgagttcgacaacgtctaa

SEQ ID NO: 56 - DNA, 888 bp, Sorghum sp.
atggctgcctccaccgcgtccaggaccaactcccgggtgaactactcgaacgagatccacgacctgtccaccgtgcagagcggctccgctgtccctaccttgttctaccctgacaaatccatcgccgacatcttcccgccgcacctcgggaagaaggtgatctcggaggtggtggcgacgttcctgctggtgttcgtgacctgcggggcggcgtccatctacggcgaggacaacaagcgcatctcgcagctggggcagtcggtggccggagggctcatcgtcaccgtcatgatctacgccaccggacacatctccggcgcgcacatgaacccagccgtcacgctctccttcgcatgcttccggcatttcccctggattcaggtgccgttctactgggcggcgcagttcacgggggcgatgtgcgcggcgttcgtgctcaaggcggtgctccaccccatcgccgtcatcggcaccaccacgccgtcgggaccgcactggcacgccctcgtcatcgagatcgtcgtcaccttcaacatgatgttcgtcacctgcgccgtcgccacggactccagggcggtgggtgagttggccgggttagcagtcggttccgcggtttgcattacgtccatcttcgcagggcctgtgtccggcggatcgatgaacccggcgaggacgctcgcgccggcggtggccagcaacgtcttcacgggactctggatctactttctcggccccgtcatcggcactctctctggagcctgggtctacacctacatccgcttcgaggaggcacccgctgccaaggacacacagcggctctcctccttcaagctccgccgcttgcagagccagtccgcgctcgccgccgacgagttcgacaccgtctaa

SEQ ID NO: 57 - DNA, 888 bp, Zea mays
atggccgccgcctccaccacgtcgaggaccaactcgcgggtgaactactcgaacgagatccacgacctctccaccgtgcagagcggctccgccgtccccaccttgttctacgacccggacaagtccatcgccgacatcttcccgccgcaccttgggaagaaggtgatctcggaggtggtggcgacgttccttctggtgttcgtcacctgcggggcggcgtccatctacggcgaggacgacaagcgcatctcgcagctggggcagtcggtggccggcgggctcatcgtcaccgtcatgatctacgccaccggccacatctccggcgcgcacatgaaccccgccgtcacgctctccttcgcatgcttccggcatttcccctggattcaggtgcccttctactgggcggcgcagttcacgggggcgatgtgcgcggcgttcgtgctcaaggcggtgctccaccccatcgccgtgatcggcaccaccacgccgtcggggccgcactggcacgcgctcctcatcgagatcgtcgtcaccttcaacatgatgttcgtcacctgcgccgtcgccaccgactccagggcggtgggtgagttggccgggttagcagtcggttccgcggtttgcattacttccatcttcgcagggccggtgtcgggcggatcgatgaacccggcgcggacgctggcgccggcggtggccagcaacgtcttcacgggcctctggatctacttcctcggccccgtcatcggcacgctctccggggcgtgggtctacacctacatccgcttcgaggaggcccccgccgccaaggacacgcagaggctctcctccttcaagctccgccgcatgcagagccagctcgccgccgacgagttcgacaccgtctaa

SEQ ID NO: 58 - DNA, 903 bp, Triticum sp
atgtcggtgacttccaacaccccgacgagggccaactcgcgagtgaactactcgaacgagatccacgacctgtccacggtgcaggacggcgcccccagcctcgcccccagcatgtactaccaggagaagtccttcgccgacttcttccctccccacctcggcaagaaggtgatatcggagatggtggcgacgttcctgctggtgttcgtgacgtgcggggcggcgtccatctacggcgccgacgtgacgcgcgtctcgcagctgggccagtccgtcgtcggcggcctcatcgtcaccgtcatgatctacgccaccgggcacatctccggcgcgcacatgaaccccgccgtcacgctctccttcgcctgcttccggcatttcccctggattcaggtgccgttctactgggcggcgcagttcaccggggcgatgtgcgcggcgttcgtgctgcgggcggtgctgcacccgatcacggtgctggggacgacgacccccacggggccgcactggcacgccctcgtcatcgagatcatcgtcaccttcaacatgatgttcatcacctgcgccgtcgccacggactcgagggcggtgggtgagttggcggggttagcagttggttccgcggtttgcattacgtccatcttcgcagggcctgtgtcaggaggatcgatgaacccggcgaggactctggcgccggcggtggccagcggcgtctacaccggcctctggatctactttctcggccccgtcatcggcaccctctccggtgcctgggtctacacctacatccgcttcgaggaggagccctccgtcaaggacggcccgcagaagctctcctcctttaagctccgccgcctgcagagccagcggtccatggccgtcgacgagtttgaccatgtctga

SEQ ID NO: 59 - DNA, 903 bp, Hordeum vulgare
atgtcggtgacttccaacacgccgacgagggccaactcgcgagtgaactactcgaacgagatccacgacctgtccacggtgcaggacggcgcccccagcctcgcccccagcatgtactaccaggagaagtcattcgccgacttcttccctccccacctcctcaagaaggtgatatcggagctggtggcgacgttcctgctggtgttcgtgacgtgcggggcggcgtccatctacggcgccgacgtgacgcgcgtctcgcagctgggccagtccgtcgtcgggggcctcatcgtcaccgtcatgatctacgccaccggacacatctccggcgcgcacatgaaccccgccgtcaccctctccttcgcctgcttccggcatttcccctggattcaggtgccgttctactgggcggcgcagttcacgggggcgatgtgcgcggcgttcgtgctgcgggcggtgctgcacccgatcacggtgctggggacgaccacgcccacggggccgcactggcacgcgctcgtcatcgagatcatcgtcaccttcaacatgatgttcatcacctgcgccgtcgccacggactcgagagcggtgggtgagttggcagggttagcagttggttccgcggtttgcattacgtccatcttcgcagggcctgtgtcaggaggatcgatgaacccggcgaggaccctggcgccggcggtggccagcggcgtctacaccggcctgtggatctactttctcggccccgtcatcggcacgctctccggcgcgtgggtctacacctacatccgcttcgaggaggagccctccgtcaaggacggcccacagaagctctcctccttcaagctccgccgcctgcagagccagcggtccatggccgtcgacgagttcgaccatgtctga

SEQ ID NO: 60 - protein, 297 aa, Sorghum sp
MSTNSRSNSRANFNNEIHDIGTVQNSTMMPPTYYDRSLADIFPPHLLKKVVSEVVSTFLLVFVTCGAAGIYGSDKDRISQLGQSVAGGLIVTVMIYAVGHISGAHMNPAVTLAFAVFRHFPWIQVPFYWAAQFTGAICASFVLKAVLHPISVLGCTTPTGPHWHSLIIEIIVTFNMMFVTLAVATDTRAVGELAGLAVGSAVCITSIFAGAVSGGSMNPARTLGPALASNLYTGLWIYFLGPVLGTLSGAWTYTYIRFEEAPSTHKDMSQKLSSFKLRRLQSQSVAAEDDELDHIQV

SEQ ID NO: 61 - protein, 295 aa, Zea mays
MSTNSRSNSRANFNNEIHDIGTAQNSSMPPTYYDRSLADIFPPHLLKKVVSEVVSTFLLVFVTCGAAGIYGSDKDRISQLGQSVAGGLIVTVMIYAVGHISGAHMNPAVTLAFAVFRHFPWIQVPFYMAAQFTGSICASFVLKAVLHPIAVLGTTTPTGPHWHSLVIEIIVTFNMMFVTLAVATDTRAVGELAGLAVGSAVCITSIFAGAVSGGSMNPARTLGPALASNLYTGLWIYFLGPVLGTLSGAWTYTYIRFEEAPSHKDMSQKLSSFKLRRLQSQSVAVDDDELDNIQV

SEQ ID NO: 62 - protein, 298 aa, Oryza sativa
MASTTAPSRTNSRVNYSNEIHDLSTVQSVSAVPSVYYPEKSFADIFPPNLLKKVISEVVATFLLVFVTCGAASIYGEDMKRISQLGQSVVGGLIVTVMIYATGHISGAHMNPAVTLSFAFFRHFPWIQVPFYWAAQFTGAMCAAFVLRAVLYPIEVLGTTTPTGPHWHALVIEIVVTFNMMFVTCAVATDSRAVGELAGLAVGSAVCITSIFAGPVSGGSMNPARTLAPAVASNVYTGLWIYFLGPVVGTLSGAWVYTYIRFEEAPAAAGGAAPQKLSSFKLRRLQSQSMAADEFDNV

SEQ ID NO: 63 - protein, 295 aa, Sorghum sp.
MAASTASRTNSRVNYSNEIHDLSTVQSGSAVPTLFYPDKSIADIFPPHLGKKVISEVVATFLLVFVTCGAASIYGEDNKRISQLGQSVAGGLIVTVMIYATGHISGAHMNPAVTLSFACFRHFPWIQVPFYWAAQFTGAMCAAFVLKAVLHPIAVIGTTTPSGPHWHALVIEIVVTFNMMFVTCAVATDSRAVGELAGLAVGSAVCITSIFAGPVSGGSMNPARTLAPAVASNVFTGLWIYFLGPVIGTLSGAWVYTYIRFEEAPAAKDTQRLSSFKLRRLQSQSALAADEFDTV

SEQ ID NO: 64 - protein, 294 aa, Zea mays
MAAASTTSRTNSRVNYSNEIHDLSTVQSGSVVPTLFYPDKSIADIFPPHLGKKVISEVVATFLLVFVTCGAASIYGEDNRRISQLGQSVAGGLIVTVNIYATGHISGAHMNPAVTLSFACFRHFPWIQVPFYWAAQFTGAMCAAFVLKAVLHPIAVIGTTTPSGPHWHALLIEIVVTFNMMFVTCAVATDSRAVGELAGLAVGSAVCITSIFAGPVSGGSMNPARTLAPAVASNVFTGLWIYFLGPVIGTLSGAWVYTYIRFEEAPAAKDTQRLSSFKLRRMQSQLAADEFDTV

SEQ ID NO: 65 - protein, 300 aa, Triticum sp.
MSVTSNTPTRANSRVNYSNEIHDLSTVQDGAPSLAPSMYYQEKSFADFFPPHLGKKVISEMVATFLLVFVTCGAASIYGADVTRVSQLGQSVVGGLIVTVMIYATGHISGAHMNPAVTLSFACFRHFPWIQVPFYWAAQFTGAMCAAFVLRAVLHPITVLGTTTPTGPHWHALVIEIIVTFNMMFITCAVATDSRAVGELAGLAVGSAVCITSIFAGPVSGGSMNPARTLAPAVASGVYTGLWIYFLGPVIGTLSGAWVYTYIRFEEEPSVKDGPQKLSSFKLRRLQSQRSMAVDEFDHV

SEQ ID NO: 66 - protein, 300 aa, Hordeum vulgare
MSVTSNTPTRANSRVNYSNEIHDLSTVQDGAPSLAPSMYYQEKSFADFFPPHLLKKVISELVATFLLVFVTCGAASIYGADVTRVSQLGQSVVGGLIVTVMIYATGHISGAHMNPAVTLSFACFRHFPWIQVPFYWAAQFTGAMCAAFVLRAVLHPITVLGTTTPTGPHWHALVIEIIVTFNMMFITCAVATDSRAVGELAGLAVGSAVCITSIFAGPVSGGSMNPARTLAPAVASGVYTGLWIYFLGPVIGTLSGAWVYTYIRFEEEPSVKDGPQKLSSFKLRRLQSQRSMAVDEFDHV

SEQ ID NO: 67 - DNA, 368 bp, Equisetum sp.
  misc_feature (7)..(7): n is a, c, g, or t
gcagctnggccagtcggtcgccggcgggctcatcgttgtngtcatgatntatgcngtcgggcacatntcncggcgcgccacatgaaccccgccgtcaccctcgccttcgccatattccacgccatttcccctggattcaggtcccgttctactgggcggcgcagttcaccggcgcgatctgcgcgtccttcgtgctcaaggcggtgctccaccccatcaccgtgatcggcaccaccgagccggtcgggccgcactggcacgcgctggtcatcgaggtcgtcgtcaccttcaacatgatgttcgtcaccctcgccgtcgccacggacaccagacgcggatgggtgagttggctgggttggctgtcggtt

SEQ ID NO: 68 - protein, 183 aa, Equisetum sp.
MATNSRSNSRATFASEIHDIGTVQNSTTPSMVYYTERSIADYFPPHLLKKVVSEVVSTFLLVFVTCGAAAISAHDVTRISQLGQAVAGGLIVVVMIYAVGHISGAHMNPAVTLTFAIFRHFPWIQVPFYWAAQFTGAICASFVLKAVLHPITVIGTTEPVGPHWHALVIEVVVTFNMMFVTLE

SEQ ID NO: 69 - DNA, 18 bp, Artificial Sequence
  Primer
tattccacgtgatcagcc

SEQ ID NO: 70 - DNA, 18 bp, Artificial Sequence
  Primer
gacgatgagggtggatgg

SEQ ID NO: 71 - DNA, 1428 bp, Triticum sp.
atgatggcgctcgcgtctctccccaaggtggtgctcggctccatcgccttcgccgtcttctggatgatggcggtgttcccgtcggtgcccttcctgcccatcggccgcacggcgggctcgctgctctccgccgtgctcatgatcgtattccacgtgatcagccccgacgacgcctacgcctccatcgacctccccatcctcggcctgctcttctccaccatggtcgtcggcggctacctcaagaacgccggcatgttcaagcacctcggcacgctcctcgcctggaagagccagggcggccgcgacctgctctgccgcgtctgcgtcgtcacggcgctcgcctcggcgcccttcaccaacgacacctgctgcgtcgtgctcaccgagttcgtgctcgagctcgccgccgagcggaacctcccggccaagcccttcctcctcgccctcgcctccagcgccaacatcggctccagcgccacccccatcggcaacccgcagaacctggtcatcgccttcaacagcaagatctccttccccaggttcctcatcggcatcctgccggccatgctcgccggcatggccgtcaacatggtcatgctgctctgcatgtactggaaggacctggagggcgtggcccccgacgcggccggcaagcagatgtcggtcgtcgaggagggcggccgctcgccgtccgtggcgtcgctcaagagcccgcacccgttcaacggcaccaccgccgacgacgggaacgagtcgatgatggaggagaacatctcgaccaagcacccgtggttcatgcagtgcacggagcaccggcgcaagctgttcctcaagagctttgcctacatcgtgacgctgggcatggtggtggcatacatggccgggctcaacatgtcgtggacggccatcaccaccgccatcgcgctcgtcgtcgtcgacttccgggacgccgagccttgcctcgtcaaggtctcctactcgctgctcgtcttcttctccggcatgttcatcacggtgagtgggttcaacaagacggggctgccgggcgccatctggaacttcatggcgccctactccaaggtggacagcgccggcggcatctccgtgctctccgtcatcatcctcctcctctccaacctcgcctccaacgtaccaacagtgctactgatggggaacgaggtggcgaccgcggcggctctgatctccccggcggcggtgactcggtcgtggctgctgttggcgtgggtgagcacggtggcgggcaacctgtcgctgctggggtcggcggcgaacctgatcgtgtgcgagcaggcgcgccgggcgccgcgcaacgcctacgagctcaccttctggaaccacctcatcttcggcgtgccatccaccctcatcgtcaccgccgtcggcatacccctcatcggcaagatctga

SEQ ID NO: 72 - protein, 475 aa, Triticum sp.
MMALASLPKVVLGSIAFAVFWMMAVFPSVPFLPIGRTAGSLLSAVLMIVFHVISPDDAYASIDLPILGLLFSTMVVGGYLKNAGMFKHLGTLLAWKSQGGRDLLCRVCVVTALASAPFTNDTCCVVLTEFVLELAAERNLPAKPFLLALASSANIGSSATPIGNPQNLVIAFNSKISFPRFLIGILPAMLAGMAVNMVMLLCMYWKDLEGVAPDAAGKQMSVVEEGGRSPSVASLKSPHPFNGTTADDGNESMMEENISTKHPWFMQCTEHRRKLFLKSFAYIVTLGMVVAYMAGLNMSWTAITTAIALVVVDFRDAEPCLVKVSYSLLVFFSGMFITVSGFNKTGLPGAIWNFMAPYSKVDSAGGISVLSVIILLLSNLASNVPTVLLMGNEVATAAALISPAAVTRSWLLLAWVSTVAGNLSLLGSAANLIVCEQARRAPRNAYELTFWNHLIFGVPSTLIVTAVGIPLIGKI

SEQ ID NO: 73 - DNA, 1455 bp, Sorghum sp
atggctctagcgtcggttgccaaggttgttcttggttccctggccttcggtgttttctgggtgcttgccgtgttcccgtcggttcccttcatgcccatcggccggactgcgggcgcgctgctgagcgcggtgctaatgatcgtgttccacgtgatcagcccggacgacgcgtacgcgtccgtggacctcccgatcctgggcctcctcttcgccaccatggtggtgggcagctacctcaagaacgccggcatgttcaagcacctgggcacgctgctggcgtggcggagccagggcggccgcgacctgctctgccgcgtctgcgtcgtcacggcgctcgccagcgcgctcttcaccaacgacacctgctgcgtcgtgctcaccgagttcgtgctcgagctcgccgccgagcgcaacctccccgctaagcccttcctgctggcgctcgcgtccagcgccaacatcggctccagcgccacgcccatcggcaacccgcagaacctcgtcatcgcattcaacagcaagatcccgttccccaagttcctgctcggaatcctgccggccatgctcgccggaatggcagtcaacatggtcatgctgctatgcatgtactggaaggacctggatgggaatgggagtcccaccatggacgttgacggcaagcggatgcaggccgtcgaggagggggctgcccatgccgccgccgccggcgtcgtggagcagagccccaagctgctgcagcttggcaccaccaacggcggcaccggctacatgtctccgctgatgactgagaacatctccaccaagcacccctggttcatgcagtgcacggagcagcggcggaagctgttcctcaagagcttcgcctacatcgtgacggtgggcatggtggtcgcctacatggtgggtctcaacatgtcgtggacggccatcaccaccgccatcgccctggtggtcgtcgacttccgcgacgccgagccgtgcctcaacaccgtctcctactcgctgctcgtcttcttctccgggatgttcatcaccgtcagcggattcaacaagacggggctgccgggggccatctggaacttcatggcgccctactccaaggtcaacagcgtcggcggcatctccgtgctctccatcatcatcctcctgctatccaacctcgcatccaacgtcccaacagtgcttctcttgggcggagaggttgcctcggcggcggcgctgatctcgccggcggcggtggttcggtcgtggctgctgctggcttgggtgagcacggtggcgggcaacctgtcgctgctggggtcggcggcgaacctgatcgtgtgcgagcaggctcggcgggcgacgcgcaacgcctacgacctcaccttctggcaacacatcgtcttcggagtgccatccaccctcatcgtcaccgccatcggcataccactcatcggaaagatcaacatctga

SEQ ID NO: 74 - protein, 484 aa, Sorghum sp.
MALASVAKVVLGSLAFGVFWVLAVFPSVPFMPIGRTAGALLSAVLMIVFHVISPDDAYASVDLPILGLLFATMVVGSYLKNAGMFKHLGTLLAWRSQGGRDLLCRVCVVTALASALFTNDTCCVVLTEFVLELAAERNLPAKPFLLALASSANIGSSATPIGNPQNLVIAFNSKIPFPKFLLGILPAMLAGMAVNMVMLLCMYWKDLDGNGSPTMDVDGKRMQAVEEGAAHAAAAGVVEQSPKLLQLGTTNGGTGYMSPLMTENISTKHPWFMQCTEQRRKLFLKSFAYIVTVGMVVAYMVGLNMSWTAITTAIALVVVDFRDAEPCLNTVSYSLLVFFSGMFITVSGFNKTGLPGAIWNFMAPYSKVNSVGGISVLSIIILLLSNLASNVPTVLLLGGEVASAAALISPAAVVRSWLLLAWVSTVAGNLSLLGSAANLIVCEQARRATRNAYDLTFWQHIVFGVPSTLIVTAIGIPLIGKINI